(19) United States
(12) Patent Application Publication
(10) Pub. No.: US 2024/0254115 A1
(43) Pub. Date: Aug. 1, 2024

(54) New Pyridine-Sulfonamide Derivatives as Sigma Ligands

(71) Applicant: ACONDICIONAMIENTO TARRASENSE, TERRASSA BARCELONA) (ES)

(72) Inventors: Carmen ALMANSA-ROSALES, BARCELONA (ES); Marina VIRGILI-BERNADÓ, BARCELONA (ES); Mónica ALONSO-XALMA, BARCELONA (ES); Ute CHRISTMANN, VILADECANS (BARCELONA) (ES)

(73) Assignee: ACONDICIONAMIENTO TARRASENSE, TERRASSA BARCELONA) (ES)

(21) Appl. No.: 18/557,933

(22) PCT Filed: Apr. 29, 2022
(86) PCT No.: PCT/EP2022/061516

§ 371 Date: Oct. 28, 2023

(30) Foreign Application Priority Data
Apr. 30, 2021 (EP) 21382387.5

(51) Int. Cl. C07D 413/14; A61K 31/4439; A61K 31/444; A61K 31/4545; A61K 31/5377; A61K 31/5386; A61K 31/541; A61K 31/553; C07D 213/71; C07D 213/74; C07D 401/12; C07D 401/14; C07D 405/14; C07D 413/04; C07D 471/10; C07D 491/107; C07D 498/08; C07D 519/00

(52) U.S. Cl. CPC C07D 413/14; A61K 31/4439; A61K 31/444; A61K 31/4545; A61K 31/5377; A61K 31/5386; A61K 31/541; A61K 31/553; C07D 213/71; C07D 213/74; C07D 401/12; C07D 401/14; C07D 405/14; C07D 413/04; C07D 471/10; C07D 491/107; C07D 498/08; C07D 519/00

(57) ABSTRACT

The present invention relates to new pyridine-sulfonamide derivatives of formula (I′) as sigma ligands having a great affinity for sigma receptors, especially the sigma-1 receptor (σ1) and/or sigma-2 receptor (σ2), as well as to the process for the preparation thereof, to compositions comprising them, and to their use as medicaments.

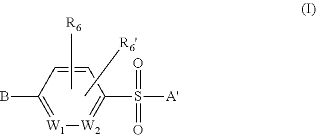

### FIELD OF THE INVENTION

The present invention relates to new pyridine-sulfonamide derivatives as sigma ligands having a great affinity for sigma receptors, especially the sigma-1 receptor (σ₁) and/or sigma-2 receptor (σ₂), as well as to the process for the preparation thereof, to compositions comprising them, and to their use as medicaments.

### BACKGROUND OF THE INVENTION

The search for new therapeutic agents has been greatly aided in recent years by better understanding of the structure of proteins and other biomolecules associated with target diseases. One important class of these proteins are the sigma (σ) receptors, originally discovered in the central nervous system (CNS) of mammals in 1976 and initially related to the dysphoric, hallucinogenic, and cardiac stimulant effects of opioids. Subsequent studies established a complete distinction between the σ receptors binding sites and the classical opiate receptors. From studies of the biology and function of sigma receptors, evidence has been presented that sigma receptor ligands may be useful in the treatment of psychosis and movement disorders such as dystonia and tardive dyskinesia, and motor disturbances associated with Huntington's chorea or Tourette's syndrome and in Parkinson's disease [Walker, J. M. et al., Pharmacological Reviews, (1990), 42, 355]. It has been reported that the known sigma receptor ligand rimcazole clinically shows effects in the treatment of psychosis [Snyder, S. H., Largent, B. L., J. Neuropsychiatry, (1989), 1, 7]. The sigma binding sites have preferential affinity for the dextrorotatory isomers of certain opiate benzomorphans, such as (+)-SKF-10047, (+)-cyclazocine, and (+)-pentazocine and also for some narcoleptics such as haloperidol. The sigma receptor has two subtypes that were initially discriminated by stereoselective isomers of these pharmacoactive drugs. (+)-SKF-10047 has nanomolar affinity for the sigma-1 (σ₁) site and has micromolar affinity for the sigma-2 (σ₂) site. Haloperidol has similar affinities for both subtypes.

The σ₁ receptor is expressed in numerous adult mammal tissues (e.g. central nervous system, ovary, testicle, placenta, adrenal gland, spleen, liver, kidney, gastrointestinal tract) as well as in embryo development from its earliest stages, and is apparently involved in a large number of physiological functions. Its high affinity for various pharmaceuticals has been described, such as for (+)-SKF-10047, (+)-pentazocine, haloperidol and rimcazole, among others, known ligands with analgesic, anxiolytic, antidepressive, antiamnesic, antipsychotic and neuroprotective activity. Hence, the σ₁ receptor has possible physiological roles in processes related to analgesia, anxiety, addiction, amnesia, depression, schizophrenia, stress, neuroprotection and psychosis [Walker, J. M. et al., Pharmacological Reviews, (1990), 42, 355; Kaiser, C. et al., Neurotransmissions, (1991), 7 (1), 1-5; Bowen, W. D., Pharmaceutica Acta Helvetiae, (2000), 74, 211-218].

The σ₁ receptor is a ligand-regulated chaperone of 223 amino acids and 25 kDa cloned in 1996 and crystallized twenty years later [Hanner, M. et al., Proc. Natl. Acad. Sci. USA, (1996), 93, 8072-8077; Su, T. P. et al., Trends Pharmacol. Sci., (2010), 31, 557-566; Schmidt, H. R. et al., Nature, (2016), 532, 527-530]. Residing primarily in the interface between the endoplasmic reticulum (ER) and mitochondrion, referred to as the mitochondria-associated membrane (MAM), it can translocate to the plasma membrane or ER-membrane and regulate the activity of other proteins by modulating N-methyl-D-aspartic (NMDA) receptors and several ion channels [Monnet, F. P. et al., Eur. J. Pharmacol., (1990), 179, 441-445; Cheng, Z. X. et al., Exp. Neurol., (2010), 210, 128-136]. Owing to the role played by the σ₁R in modulating pain-related hypersensitivity and sensitization phenomena, σ₁R antagonists have been also proposed for the treatment of neuropathic pain [Drews, E. et al., Pain, 2009, 145, 269-270; De la Puente, B. et al., Pain (2009), 145, 294-303; Diaz, J. L. et al., J. Med. Chem., (2012), 55, 8211-8224; Romero et al., Brit. J. Pharm., (2012), 166, 2289-2306; Merlos, M. et al., Adv. Exp. Med. Biol., (2017), 964, 85-107]. Additionally, the σ₁ receptor has been known to modulate opioid analgesia, and the relationship between the μ-opioid and σ₁ receptors has been shown to involve direct physical interaction, which explains why σ₁ receptor antagonists enhance the antinociceptive effect of opioids without increasing their adverse effects [Chien, C. C. et al, J. Pharmacol. Exp. Ther., (1994), 271, 1583-1590; King, M. et al, Eur. J. Pharmacol., (1997), 331, R5-6; Kim, F. J. et al., Mol. Pharmacol., (2010), 77, 695-703; Zamanillo, D. et al., Eur. J. Pharmacol., (2013), 716, 78-93].

The σ₂ receptor was initially identified by radioligand binding as a site with high affinity for di-o-tolylguanidine (DTG) and haloperidol [Hellewell, S. B. et al., Brain Res., (1990), 527, 244-253]. Two decades later, progesterone receptor membrane component 1 (PGRMC1), a cytochrome-related protein that binds directly to heme and regulates lipid and drug metabolism and hormone signaling, was proposed as the complex where resides the σ₂R binding site [Xu, J. et al., Nat. Commun., (2011), 2, 380]. Finally, in 2017, the σ₂R subtype was purified and identified as transmembrane protein-97 (TMEM97), an endoplasmic-reticulum-resident molecule implicated in cholesterol homeostasis due to its association with the lysosomal Niemann-Pick cholesterol transporter type 1 (NPC1) [Alon, A. et al., Proc. Natl. Acad. Sci. USA, (2017), 114, 7160-7165; Ebrahimi-Fakhari, D. et al., Human Molecular Genetics, (2016), 25, 3588-3599]. The role of σ₂ receptor in cholesterol pathways was known since the 1990s and recent studies published by Mach et al. on modulation of trafficking and internalization of LDL by the formation of a ternary complex between LDLR, PGRMC1 and TMEM97, reinforces this association [Moebius, F. F. et al., Trends Pharmacol. Sci., (1997), 18, 67-70; Riad, A. et al., Sci. Rep., (2018), 8, 16845].

σ₂R/TMEM97, previously known also as meningioma-associated protein, MAC30, is expressed in various normal and diseased human tissues and up-regulation in certain tumors and down-regulation in other suggested that this protein played a distinct role in human malignancies. The cloning of σ₂ receptor confirmed its overexpression in epithelial, colorectal, ovarian lung and breast cancers [Moparthi, S. B. et al., Int. J. Oncol., (2007), 30, 91-95; Yan, B. Y. et al., Chemotherapy, (2010), 56, 424-428; Zhao, Z. R.; Chemotherapy, (2011), 57, 394-401; Ding, H. et al., Asian Pac. J. Cancer Prev., (2016), 17, 2705-2710]. σ₂R/TMEM97 has a molecular weight of 18-21.5 kDa and its sequence predicts a four transmembrane domain protein with cytosolic N and C terminal [Hellewell, S. B. et al., Eur. J. Pharmacol. Mol. Pharmacol. Sect., (1994), 268, 9-18]. The potential signal transduction of σ₂ receptor is not yet understood, but it seems to modulate Ca²⁺ and K⁺ channels, and to interact with caspases, epidermal growth factor receptor (EGFR), and with mammalian target of rapamycin, mTOR, signaling pathways [Vilner, B. J. et al., J. Pharmacol. Exp. Ther., (2000), 292, 900-911; Wilke, R. A. et al., J. Biol. Chem., (1999), 274, 18387-18392; Huang, Y.-S. et al., Med. Res. Rev., (2014), 34, 532-566]. These findings would explain the apoptotic effect of some σ₂ ligands by lysosome dysfunction, reactive oxygen species (ROS) production and caspase-dependent events [Ostenfeld, M. S. et al., Autophagy, (2008), 4, 487-499; Hornick, J. R. et al., J. Exp. Clin. Cancer Res., (2012), 31, 41; Zeng, C. et al., Br. J. Cancer, (2012), 106, 693-701; Pati, M. L. et al., BMC Cancer, (2017), 17, 51].

The σ₂ receptor is involved also in dopaminergic transmission, microglia activation, and neuroprotection [Guo, L. et al., Curr. Med. Chem. (2015), 22, 989-1003]. Terada et al. published in 2018 that σ₂ ligands enhance nerve growth factor (NGF)-induced neurite outgrowth in PC12 cells [Terada, K. et al., Plos One, (2018), 13, e0209250]. The σ₂ receptor plays a key role in amyloid β (Aβ)-induced synaptotoxicity, and σ₂ receptor ligands that block the interaction of Aβ oligomers with the σ₂ receptor have been shown to be neuroprotective [Izzo, N. J. et al., Plos One, (2014), 9, e111899]. σ₂ receptor modulators improve cognitive performance in a transgenic mouse model of Alzheimer's disease (AD), and in two mouse traumatic brain injury models, and could also reduce ischemic stroke injury by enhancing glial cell survival, blocking ischemia-induced glial cell activation, and decreasing nitrosative stress [Katnik, C. et al., J. Neurochem., (2016), 139, 497-509; Yi, B. et al., J. Neurochem., (2017), 140, 561-575; Vázquez-Rosa, E. et al., ACS Chem. Neurosci., (2019), 10, 1595-1602]. The σ₂ receptor has been implicated in other neurological disorders as schizophrenia [Harvey, P. D. et al., Schizophrenia Research (2020), 215, 352-356], alcohol abuse [Scott, L. L. et al., Neuropsychopharmacology, (2018), 43, 1867-1875] and pain [Sahn, J. J. et al., ACS Chem. Neurosci., (2017), 8, 1801-1811]. Norbenzomorphan UKH-1114, a σ₂ ligand, relieved mechanical hypersensitivity in the spared nerve injury (SNI) mice model of neuropathic pain, an effect explained by the preferential expression of σ₂R/TMEM97 gene in structures involved in pain such as the dorsal root ganglion (DRG).

The σ₂ receptor requires two acidic groups (Asp29, Asp56) for ligand binding, similar to σ₁R, which requires Asp126 and Glu172. σ₁R and σ₂R might have similarities in their binding sites but not necessarily other structural similarities if their amino acid sequences are compared. As σ₁R, σ₂ receptor interacts with a wide range of signaling proteins, receptors and channels, but the question if σ₂ receptor has a primarily structural or a modulatory activity remains to be answered. Several classes of σ₂ receptor ligands have been developed since Perregaard et al., synthesized Siramesine and indole analogues in 1995 [Perregaard, J. et al., J. Med. Chem., (1995), 38, 1998-2008]: tropanes [Bowen, W. D. et al., Eur. J. Pharmacol., (1995), 278, 257-260], norbenzomorphans [Sahn, J. J. et al., ACS Med. Chem. Lett., (2017), 8, 455-460], tetrahydroisoquinolines [Sun, Y.-T. et al., Eur. J. Med. Chem., (2018), 147, 227-237] or isoindolines [Grundmana, M. et al., Alzheimer's & Dementia: Translational Research & Clinical Interventions, (2019), 5, 20-26] amongst others [Berardi, F. et al., J. Med. Chem., (2004), 47, 2308-2317]. Many of these ligands present a lack of selectivity over serotoninergic receptors but mainly, there is a difficulty to reach a high selectivity over σ₁. Several σ₁-selective ligands are available, but ligands with high selectivity for σ₂ over σ₁ are relatively scarce. A significant challenge for the study of the σ₂ receptor is the paucity of highly σ₂-selective ligands.

In view of the potential therapeutic applications of agonists or antagonists of the sigma receptor, a great effort has been directed to find selective ligands. Thus, the prior art has disclosed different sigma receptor ligands, as outlined above.

Nevertheless, there is still a need to find compounds having pharmacological activity towards the sigma receptor, being both effective, selective, and/or having good “drugability” properties, i.e. good pharmaceutical properties related to administration, distribution, metabolism and excretion.

Surprisingly, it has been observed that the new pyridine-sulfonamide derivatives with general Formula (I) show a selective affinity for sigma receptors, in particular, for σ₁ and σ₂. These compounds are therefore particularly suitable as pharmacologically active agents in medicaments for the prophylaxis and/or treatment of disorders or diseases related to sigma receptors.

### SUMMARY OF THE INVENTION

The present invention discloses novel compounds with great affinity to sigma receptors which might be used for the treatment of sigma related disorders or diseases. In particular, the compounds of the invention can be useful for the treatment of pain and pain related disorders and/or CNS (Central Nervous System) disorders.

The invention is directed in a main aspect to a compound of Formula (I′),

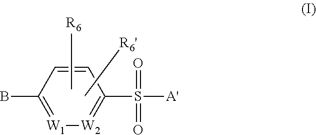

wherein A, B, W₁, W₂, R₁, R₁′, R₆ and R₆′ are as defined below in the detailed description.

A further aspect of the invention refers to the processes for preparation of compounds of formula (I′).

It is also an aspect of the invention a pharmaceutical composition comprising a compound of formula (I′).

Finally, it is an aspect of the invention a compound of formula (I′) for use in therapy and more particularly for the treatment of pain and pain related conditions and/or CNS (Central Nervous System) disorders.

### DETAILED DESCRIPTION OF THE INVENTION

The invention is directed to a family of compounds, in particular to pyridine-sulfonamide derivatives which show a pharmacological activity towards the sigma receptors, thus solving the above problem of identifying alternative or improved pain and/or CNS treatments by offering such compounds.

The applicant has found that the problem of providing a new effective and alternative solution for treating pain and pain related disorders and/or CNS (Central Nervous System) disorders can surprisingly be solved by using compounds binding to the sigma receptors.

In a first aspect, the present invention is directed to a compound of formula (I′):

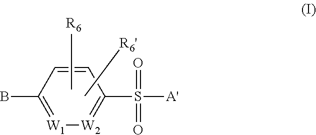

wherein:
A is one of the following moieties:

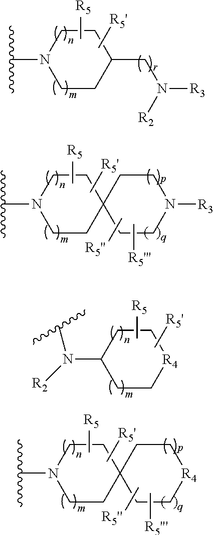

B represents a —NR₁R₁, group; a branched or unbranched C₁₋₆ alkyl radical; a branched or unbranched C₁₋₆ haloalkyl radical; an heterocycloalkyl radical containing at least an O as heteroatom; or an heteroaryl radical containing at least an N as heteroatom optionally substituted by a C₁₋₆ haloalkyl radical;
W₁ and W₂ independently represent —N— or —CH— with the proviso that one of W₁ or W₂ is —N— and the other is —CH—;
R₁ and R₁′ independently represent a hydrogen atom; a branched or unbranched C₁₋₆ alkyl radical; or a —C(O)R where R represents a C₁₋₆ alkyl radical, with the proviso that at least one of R₁ and R₁′ is different from a hydrogen atom; or alternatively R₁ and R₁′ form together with the nitrogen atom to which they are attached a 5- to 12-membered N-containing heterocyclic ring or ring system that may further contain one or more additional heteroatoms selected from N, O or S and that are optionally substituted by one or more Rₐ radicals;
Each Rₐ is independently a hydrogen atom; a branched or unbranched C₁₋₆ alkyl radical; a halogen atom, a ON group, a C₁₋₆ haloalkyl radical, a OH group; a carbonyl group or a C₁₋₆ alkoxy radical;
R₂ is a hydrogen atom or a branched or unbranched C₁₋₆ alkyl radical;
R₃ is a branched or unbranched C₃₋₁₀ alkyl radical; an alkylaryl radical; or an alkylheteroaryl radical; wherein the aryl and the heteroaryl groups may be substituted by one or more R₃′ radicals;
R₃′ is a branched or unbranched C₁₋₆alkyl radical, a halogen atom, a CN group, a C₁₋₆ haloalkyl radical, a OH group or a C₁₋₆ alkoxy radical;
R₄ is —CH—(NR₂R₃) or —NR₂R₃;
R₅, R₅′, R₅″ and R₅′″ are independently from one another a hydrogen atom or a branched or unbranched C₁₋₆ alkyl radical; a C₁₋₆ alkoxy radical; a halogen atom; a C₁₋₆ haloalkyl group; a OH group or a CN group;
R₆ and R₆′ are independently from one another a hydrogen atom or a branched or unbranched C₁₋₆ alkyl radical; a C₁₋₆ alkoxy radical; a halogen atom; a C₁₋₆ haloalkyl group; a OH group or a CN group;
n is 0, 1 or 2;
m is 0, 1 or 2;
p is 0, 1 or 2;
q is 0, 1 or 2;
r is 0, 1 or 2;
with the proviso that when R₃ is a benzyl group R₃′ is not an a C₁₋₆ alkoxy radical;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt, co-crystal or prodrug thereof, or a corresponding solvate thereof.

Unless otherwise stated, the compounds of the invention are also meant to include isotopically-labelled forms i.e. compounds which differ only in the presence of one or more isotopically-enriched atoms. For example, compounds having the present structures except for the replacement of at least one hydrogen atom by a deuterium or tritium, or the replacement of at least one carbon by ¹³C or ¹⁴C-enriched carbon, or the replacement of at least one nitrogen by ¹⁵N-enriched nitrogen are within the scope of this invention.

The compounds of general formula (I) or their salts or solvates are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts, solvates or prodrugs.

For the sake of clarity the expression “a compound according to formula (I), wherein R₁, R₂, etc are as defined below in the detailed description” would Oust like the expression “a compound of formula (I) as defined in the claims”) refer to “a compound according to formula (I)”, wherein the definitions of the respective substituents R₁, R₂, etc. (also from the cited claims) are applied.

For clarity purposes, all groups and definitions described in the present description and referring to compounds of formula (I), also apply to all intermediates of synthesis.

“Halogen” or “halo” as referred in the present invention represent fluorine, chlorine, bromine or iodine. When the term “halo” is combined with other substituents, such as for instance “C₁₋₆ haloalkyl” or “C₁₋₆ haloalkoxy” it means that the alkyl or alkoxy radical can respectively contain at least one halogen atom.

“C₁₋₆ alkyl”, as referred to in the present invention, are saturated aliphatic radicals. They may be unbranched (linear) or branched and are optionally substituted. C₁₋₆-alkyl as expressed in the present invention means an alkyl radical of 1, 2, 3, 4, 5 or 6 carbon atoms. Preferred alkyl radicals according to the present invention include but are not restricted to methyl, ethyl, propyl, n-propyl, isopropyl, butyl, n-butyl, tert-butyl, isobutyl, sec-butyl, 1-methylpropyl, 2-methylpropyl, 1,1-dimethylethyl, pentyl, n-pentyl, 1,1-dimethylpropyl, 1,2-dimethylpropyl, 2,2-dimethylpropyl, 3,3-dimethylbutyl, hexyl, 1-methylpentyl. The most preferred alkyl radical are C₁₋₆alkyl, such as methyl, ethyl, propyl, n-propyl, isopropyl, butyl, n-butyl, tert-butyl, isobutyl, sec-butyl, isopentyl, 1-methylpropyl, 2-methylpropyl, 1,1-dimethylethyl or 3,3-dimethylbutyl. Alkyl radicals, as defined in the present invention, are optionally mono- or polysubstituted by substitutents independently selected from a halogen, branched or unbranched C₁₋₆-alkoxy, branched or unbranched C₁₋₆-alkyl, C₁₋₆-haloalcoxy, C₁₋₆-haloalkyl, trihaloalkyl or a hydroxyl group.

“C₁₋₆ alkoxy” as referred to in the present invention, is understood as meaning an alkyl radical as defined above attached via oxygen linkage to the rest of the molecule. Examples of alkoxy include, but are not limited to methoxy, ethoxy, propoxy, butoxy or tert-butoxy.

“C₃₋₉ Cycloalkyl” as referred to in the present invention, is understood as meaning saturated and unsaturated (but not aromatic), cyclic hydrocarbons having from 3 to 9 carbon atoms which can optionally be unsubstituted, mono- or polysubstituted. Examples for cycloalkyl radical preferably include but are not restricted to cyclopropyl, cyclobutyl, cyclopentyl, cyclohexyl. Cycloalkyl radicals, as defined in the present invention, are optionally mono- or polysubstituted by substitutents independently selected from a halogen atom, branched or unbranched C₁₋₆-alkyl, branched or unbranched C₁₋₆-alkoxy, C₁₋₆-haloalcoxy, C₁₋₆-haloalkyl, trihaloalkyl or a hydroxyl group.

A alkylcycloalkyl group/radical, as defined in the present invention, comprises a branched or unbranched, optionally at least mono-substituted alkyl chain of 1 to 6 atoms which is bonded to a cycloalklyl group, as defined above. The alkylcycloalkyl radical is bonded to the molecule through the alkyl chain. A preferred alkylcycloalkyl group/radical is a cyclopropylmethyl group or a cyclopentylpropyl group, wherein the alkyl chain is optionally branched or substituted. Preferred substituents for alkyl cycloalkylgroup/radical, according to the present invention, are independently selected from a halogen atom, branched or unbranched C₁₋₆-alkyl, branched or unbranched C₁₋₆-alkoxy, C₁₋₆-haloalcoxy, C₁₋₆-haloalkyl, trihaloalkyl or a hydroxyl group.

A heterocyclyl radical or group (also called heterocyclyl hereinafter) is understood as meaning 4 to 18 membered mono or fused polycyclic heterocyclic ring systems, with at least one saturated or unsaturated ring which contains one or more heteroatoms selected from the group consisting of nitrogen, oxygen and/or sulfur in the ring. A heterocyclic group can also be substituted once or several times.

Subgroups inside the heterocyclyls as understood herein include heteroaryls and non-aromatic heterocyclyls.
the heteroaryl (being equivalent to heteroaromatic radicals or aromatic heterocyclyls) is an aromatic 5 to 18 membered mono or fused polycyclic heterocyclic ring system, including spirofused ring systems, of one or more rings of which at least one aromatic ring contains one or more heteroatoms from the group consisting of nitrogen, oxygen and/or sulfur in the ring; preferably it is a 5 to 18 membered mono or fused polycyclic aromatic heterocyclic ring system of one or two rings of which at least one aromatic ring contains one or more heteroatoms selected from the group consisting of nitrogen, oxygen and/or sulfur in the ring; more preferably it is selected from furan, benzofuran, thiophene, benzothiophene, pyrrole, pyridine, pyrimidine, pyrazine, quinoline, isoquinoline, phthalazine, benzothiazole, indole, benzotriazole, carbazole, quinazoline, thiazole, imidazole, pyrazole, oxazole, isoxazole, dihydro-4H-pyrano[3,4-d]isoxazole, oxadiazole, thiophene, dihydro-4H-pyrano[3,4-d]isoxazole and benzimidazole;
the non-aromatic heterocyclyl is a 4 to 18 membered mono or fused polycyclic heterocyclic ring system, including spiro fused ring systems, of one or more rings of which at least one ring—with this (or these) ring(s) then not being aromatic—contains one or more heteroatoms from the group consisting of nitrogen, oxygen and/or sulfur in the ring; preferably it is a 4 to 18 membered mono or fused polycyclic heterocyclic ring system of one or two rings of which one or both rings—with this one or two rings then not being aromatic—contain/s one or more heteroatoms selected from the group consisting of nitrogen, oxygen and/or sulfur in the ring, more preferably it is selected from azetidine, oxetane, tetrahydrofuran, azepan, oxazepan, pyrrolidine, piperidine, piperazine, tetrahydropyran, morpholine, indoline, oxopyrrolidine, benzodioxane, (1S,4S)-2-oxa-5-azabicyclo[2.2.1]heptane, (1R,5S)-3-oxa-8-azabicyclo[3.2.1]octane, 1-oxa-8-azaspiro[4.5]decane, 2,6-diazaspiro[3.4]octane 2,7-diazaspiro[3.5]nonane, 2,8-diazaspiro[4.5]decane, 3,9-diazaspiro[5.5]undecane, 2,9-diazaspiro[5.5]undecane, 6-azaspiro[3.4]octane, 7-azaspiro[3.5]nonane, 3-azaspiro[5.5]undecane, especially is azepan, oxazepan, (1S,4S)-2-oxa-5-azabicyclo[2.2.1]heptane, (1R,5S)-3-oxa-8-azabicyclo[3.2.1]octane, 1-oxa-8-azaspiro[4.5]decane pyrrolidine, piperidine, 2,6-diazaspiro[3.4]octane, 2,7-diazaspiro[3.5]nonane, 2,8-diazaspiro[4.5]decane, 3,9-diazaspiro[5.5]undecane, 2,9-diazaspiro[5.5]undecane, 6-azaspiro[3.4]octane, 7-azaspiro[3.5]nonane, 3-azaspiro[5.5]undecane, benzodioxane and morpholine.

Preferably, in the context of this invention heterocyclyl is defined as a 4 to 18 membered mono or fused polycyclic ring system, including spirofused ring systems, of one or more saturated or unsaturated rings of which at least one ring contains one or more heteroatoms selected from the group consisting of nitrogen, oxygen and/or sulfur in the ring. Preferably it is a 4 to 18 membered mono or fused polycyclic heterocyclic ring system of one or two saturated or unsaturated rings of which at least one ring contains one or more heteroatoms selected from the group consisting of nitrogen, oxygen, and sulfur in the ring. More preferably, it is a 4 to 12 membered mono or bicyclic heterocyclyl ring system containing one nitrogen atom and optionally a second heteroatom selected from nitrogen and oxygen. In another preferred embodiment of the invention, said heterocyclyl is a substituted mono or bicyclic heterocyclyl ring system.

Preferred examples of heterocyclyls include azetidine, azepane, oxazepan, pyrrolidine, piperidine, oxetane, tetrahydrofuran, imidazole, oxadiazole, tetrazole, pyridine, pyrimidine, piperazine, benzofuran, benzimidazole, indazole, benzodiazole, thiazole, benzothiazole, tetrahydropyran, morpholine, indoline, furan, triazole, isoxazole, pyrazole, thiophene, benzothiophene, pyrrole, pyrazine, pyrrolo[2,3b]pyridine, quinoline, isoquinoline, tetrahydroisoquinoline, phthalazine, benzo-1,2,5-thiadiazole, indole, benzotriazole, benzoxazole oxopyrrolidine, pyrimidine, benzodioxolane, benzodioxane, carbazole, quinazoline, 2,6-diazaspiro[3.4]octane 2,7-diazaspiro[3.5]nonane, 2,8-diazaspiro[4.5]decane, 3,9-diazaspiro[5.5]undecane, 2,9-diazaspiro[5.5]undecane, 6-azaspiro[3.4]octane, 7-azaspiro[3.5]nonane, 3-azaspiro[5.5]undecane octahydropyrrolo[3,4-c]pyrrole, especially is pyridine, piperazine, pyrazine, indazole, benzodioxane, thiazole, benzothiazole, morpholine, tetrahydropyran, pyrazole, imidazole, piperidine, thiophene, indole, benzimidazole, pyrrolo[2,3-b]pyridine, benzoxazole, oxopyrrolidine, pyrimidine, oxazepane, pyrrolidine, azetidine, azepane, oxazepane, oxetane, tetrahydrofuran, 2,6-diazaspiro[3.4]octane 2,7-diazaspiro[3.5]nonane, 2,8-diazaspiro[4.5]decane, 3,9-diazaspiro[5.5]undecane, 2,9-diazaspiro[5.5]undecane, 6-azaspiro[3.4]octane, 7-azaspiro[3.5]nonane, 3-azaspiro[5.5]undecane.

An N-containing heterocyclyl is a heterocyclic ring system of one or more saturated or unsaturated rings of which at least one ring contains a nitrogen and optionally one or more further heteroatoms selected from the group consisting of nitrogen, oxygen and/or sulfur in the ring; preferably is a heterocyclic ring system of one or two saturated or unsaturated rings of which at least one ring contains a nitrogen and optionally one or more further heteroatoms selected from the group consisting of nitrogen, oxygen and/or sulfur in the ring, more preferably is selected from azetidine, azepane, oxazepam, pyrrolidine, imidazole, oxadiazole, tetrazole, azetidine, pyridine, pyrimidine, piperidine, piperazine, benzimidazole, indazole, benzothiazole, benzodiazole, morpholine, indoline, triazole, isoxazole, pyrazole, pyrrole, pyrazine, pyrrolo[2,3-b]pyridine, quinoline, quinolone, isoquinoline, tetrahydrothienopyridine, phthalazine, benzo-1,2,5-thiadiazole, indole, benzotriazole, benzoxazole oxopyrrolidine, carbazole, thiazole, (1S,4S)-2-oxa-5-azabicyclo[2.2.1]heptane, (1R,5S)-3-oxa-8-azabicyclo[3.2.1]octane, 1-oxa-8-azaspiro[4.5]decane, 2,6-diazaspiro[3.4]octane 2,7-diazaspiro[3.5]nonane, 2,8-diazaspiro[4.5]decane, 3,9-diazaspiro[5.5]undecane, 2,9-diazaspiro[5.5]undecane, 6-azaspiro[3.4]octane, 7-azaspiro[3.5]nonane, 3-azaspiro[5.5]undecane or octahydropyrrolo[3,4-c]pyrrole.

In connection with aromatic heterocyclyls (heteroaryls), non-aromatic heterocyclyls, aryls and cycloalkyls, when a ring system falls within two or more of the above cycle definitions simultaneously, then the ring system is defined first as an aromatic heterocyclyl (heteroaryl) if at least one aromatic ring contains a heteroatom. If no aromatic ring contains a heteroatom, then the ring system is defined as a non-aromatic heterocyclyl if at least one non-aromatic ring contains a heteroatom. If no non-aromatic ring contains a heteroatom, then the ring system is defined as an aryl if it contains at least one aryl cycle. If no aryl is present, then the ring system is defined as a cycloalkyl if at least one non-aromatic cyclic hydrocarbon is present.

“Heterocycloalkyl” as referred to in the present invention, are understood as meaning saturated and unsaturated (but not aromatic), generally 5 or 6 membered cyclic hydrocarbons which can optionally be unsubstituted, mono- or polysubstituted and which have at least one heteroatom in their structure selected from N, O or S. Examples for heterocycloalkyl radical preferably include but are not restricted to pyrroline, pyrrolidine, pyrazoline, aziridine, azetidine, tetrahydropyrrole, oxirane, oxetane, dioxetane, tetrahydropyrane, tetrahydrofurane, dioxane, dioxolane, oxazolidine, piperidine, piperazine, morpholine, azepane or diazepane. Heterocycloalkyl radicals, as defined in the present invention, are optionally mono- or polysubstituted by substitutents independently selected from a halogen atom, branched or unbranched C₁₋₆-alkyl, branched or unbranched C₁₋₆-alkoxy, C₁₋₆-haloalkoxy, C₁₋₆-haloalkyl, trihaloalkyl or a hydroxyl group. More preferably heterocycloalkyl in the context of the present invention are 5 or 6-membered ring systems optionally at least monosubstituted.

An alkylheterocycloalkyl group/radical, as defined in the present invention, comprises a linear or branched, optionally at least mono-substituted alkyl chain of 1 to 6 atoms which is bonded to a cycloalklyl group, as defined above. The alkylheterocycloalkyl radical is bonded to the molecule through the alkyl chain. A preferred alkyheterocycloalkyll group/radical is a piperidinethyl group or a piperazinylmethyl group, wherein the alkyl chain is optionally branched or substituted. Preferred substituents for alkylheterocycloalkyl group/radical, according to the present invention, are independently selected from a halogen atom, branched or unbranched C₁₋₆-alkyl, branched or unbranched C₁₋₆-alkoxy, C₁₋₆-haloalcoxy, C₁₋₆-haloalkyl, trihaloalkyl or a hydroxyl group.

“Aryl” as referred to in the present invention, is understood as meaning ring systems with at least one aromatic ring but without heteroatoms even in only one of the rings. These aryl radicals may optionally be mono-or polysubstituted by substitutents independently selected from a halogen atom, —ON, branched or unbranched C₁₋₆-alkyl, branched or unbranched C₁₋₆-alkoxy, C₁₋₆-haloalcoxy, C₁₋₆-haloalkyl, a heterocyclyl group and a hydroxyl group. Preferred examples of aryl radicals include but are not restricted to phenyl, naphthyl, fluoranthenyl, fluorenyl, tetralinyl, indanyl or anthracenyl radicals, which may optionally be mono- or polysubstituted, if not defined otherwise. More preferably aryl in the context of the present invention is a 6-membered ring system optionally at least mono or polysubstituted.

An alkylaryl radical, as defined in the present invention, comprises an unbranched or branched, optionally at least mono-substituted alkyl chain of 1 to 6 carbon atoms which is bonded to an aryl group, as defined above. The alkylaryl radical is bonded to the molecule through the alkyl chain. A preferred alkylaryl radical is a benzyl group or a phenetyl group, wherein the alkyl chain is optionally branched or substituted. Preferred substituents for alkylaryl radicals, according to the present invention, are independently selected from a halogen atom, branched or unbranched C₁₋₆-alkyl, branched or unbranched C₁₋₆-alkoxy, C₁₋₆-haloalcoxy, C₁₋₆-haloalkyl, trihaloalkyl or a hydroxyl group.

An alkylheteroaryl group/radical as defined in the present invention, comprises a linear or branched, optionally at least mono-substituted alkyl chain of 1 to 6 carbon atoms which is bonded to an heteroaryl group, as defined above. The alkylheteroaryl radical is bonded to the molecule through the alkyl chain. A preferred alkylheteroaryl radical is a piridinylmethyl group, wherein the alkyl chain is optionally branched or substituted. Preferred substituents for alkylheteroaryl radicals, according to the present invention, are independently selected from a halogen atom, branched or unbranched C₁₋₆-alkyl, branched or unbranched C₁₋₆-alkoxy, C₁₋₆-haloalcoxy, C₁₋₆-haloalkyl, trihaloalkyl or a hydroxyl group.

The term “condensed” according to the present invention means that a ring or ring-system is attached to another ring or ring-system, whereby the terms “annulated” or “annelated” are also used by those skilled in the art to designate this kind of attachment.

The term “ring system” according to the present invention refers to a system consisting of at least one ring of connected atoms but including also systems in which two or more rings of connected atoms are joined with “joined” meaning that the respective rings are sharing one (like a spiro structure), two or more atoms being a member or members of both joined rings. The “ring system” thus defined comprises saturated, unsaturated or aromatic carbocyclic rings which contain optionally at least one heteroatom as ring member and which are optionally at least mono-substituted and may be joined to other carbocyclic ring systems such as aryl radicals, heteroaryl radicals, cycloalkyl radicals etc.

The terms “condensed”, “annulated” or “annelated” are also used by those skilled in the art to designate this kind of join.

A leaving group is a group that in a heterolytic bond cleavage keeps the electron pair of the bond. Suitable leaving groups are well known in the art and include Cl, Br, I and —O—SO₂R¹⁴, wherein R¹⁴ is F, C₁₋₄-alkyl, C₁₋₄-haloalkyl, or optionally substituted phenyl. The preferred leaving groups are Cl, Br, I, tosylate, mesylate, triflate, nonaflate and fluorosulphonate.

“Protecting group” is a group that is chemically introduced into a molecule to avoid that a certain functional group from that molecule undesirably reacts in a subsequent reaction. Protecting groups are used, among others, to obtain chemoselectivity in chemical reactions. The preferred protecting group in the context of the invention are Boc (tert-butoxycarbonyl) or Teoc (2-(trimethylsilyl)ethoxycarbonyl).

The term “salt” is to be understood as meaning any form of the active compound according to the invention in which this assumes an ionic form or is charged and is coupled with a counter-ion (a cation or anion). The definition particularly includes physiologically acceptable salts, this term must be understood as equivalent to “pharmaceutically acceptable salts”.

The term “pharmaceutically acceptable salts” in the context of this invention means any salt that is tolerated physiologically (normally meaning that it is not toxic, particularly as a result of the counter-ion) when used in an appropriate manner for a treatment, particularly applied or used in humans and/or mammals. This definition specifically includes in the context of this invention a salt formed by a physiologically tolerated acid, i.e. salts of a specific active compound with physiologically tolerated organic or inorganic acids—particularly when used on humans and/or mammals. Examples of this type of salts are those formed with: hydrochloric acid, hydrobromic acid, sulphuric acid, methanesulfonic acid, formic acid, acetic acid, oxalic acid, succinic acid, malic acid, tartaric acid, mandelic acid, fumaric acid, lactic acid or citric acid. In addition, the pharmaceutically acceptable salts may be formed with a physiologically tolerated cation, preferably inorganic, particularly when used on humans and/or mammals. Salts with alkali and alkali earth metals are particularly preferred, as well as those formed with ammonium cations (NH₄⁺). Preferred salts are those formed with (mono) or (di)sodium, (mono) or (di)potassium, magnesium or calcium. These physiologically acceptable salts may also be formed with anions or acids and, in the context of this invention, are understood as being salts formed by at least one compound used in accordance with the invention—normally protonated, for example in nitrogen—such as a cation and at least one physiologically tolerated anion, particularly when used on humans and/or mammals.

The compounds of the invention may be present in crystalline form or in amorphous form.

Any compound that is a solvate of a compound according to formula (I) defined above is understood to be also covered by the scope of the invention. Methods of solvation are generally known within the art. Suitable solvates are pharmaceutically acceptable solvates. The term “solvate” is to be understood as meaning any form of the active compound according to the invention in which this compound has attached to it via non-covalent binding another molecule (most likely a polar solvent) especially including hydrates and alcoholates, like methanolate or ethanolate.

The term “co-crystal” is to be understood as a crystalline material comprising a specific active compound with at least one additional component, usually a co-crystal former, and of which at least two of the constituents are held together by weak interactions. Weak interaction is being defined as an interaction which is neither ionic nor covalent and includes for example: hydrogen bonds, van der Waals forces, and π-π interactions.

The term “prodrug” is used in its broadest sense and encompasses those derivatives that are converted in vivo to the compounds of the invention. Such derivatives would readily occur to those skilled in the art, and include, depending on the functional groups present in the molecule and without limitation, the following derivatives of the compounds of the invention: esters, amino acid esters, phosphate esters, metal salts sulfonate esters, carbamates, and amides. Examples of well-known methods of producing a prodrug of a given acting compound are known to those skilled in the art and can be found e.g. in Krogsgaard-Larsen et al. “Textbook of Drug design and Discovery” Taylor & Francis (April 2002).

Any compound that is a prodrug of a compound of general formula (I) is within the scope of the invention. Particularly favored prodrugs are those that increase the bioavailability of the compounds of this invention when such compounds are administered to a patient (e.g., by allowing an orally administered compound to be more readily absorbed into the blood) or which enhance delivery of the parent compound to a biological compartment (e.g., the brain or lymphatic system) relative to the parent species.

Any compound that is a N-oxide of a compound according to the invention like a compound according to formula (I) defined above is understood to be also covered by the scope of the invention.

The compounds of formula (I) as well as their salts or solvates are preferably in pharmaceutically acceptable or substantially pure form. By pharmaceutically acceptable pure form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts. This applies also to its solvates or prodrugs.

Unless otherwise defined, all the groups above mentioned that can be substituted or unsubstituted may be substituted at one or more available positions by one or more suitable groups such as a halogen, preferably Cl or F; OR′, ═O, SR′, SOR′, SO₂R′, OSO₂R′, OSO₃R′, NO₂, NHR′, NR′R″, ═N—R′, N(R′)COR′, N(COR′)₂, N(R′)SO₂R′, N(R′)C(═NR′)N(R′)R′, N₃, CN, halogen, COR′, COOR′, OCOR′, OCOOR′, OCONHR′, OCONR′R″, CONHR′, CONR′R″, CON(R′)OR′, CON(R′)SO₂R′, PO(OR′)₂, PO(OR′)R′, PO(OR′)(N(R′)R′), C₁₋₆ alkyl, C₃₋₁₀ cycloalkyl, C₂₋₆ alkenyl, C₂₋₆ alkynyl, aryl, and heterocyclic group, wherein each of the R′ and R″ groups is independently selected from the group consisting of hydrogen, C₁₋₆ alkyl, C₃₋₁₀ cycloalkyl, C₂₋₆ alkenyl, C₂₋₆ alkynyl, aryl and heterocyclic group. Where such groups are themselves substituted, the substituents may be chosen from the foregoing list.

In a particular and preferred embodiment of the invention compound of formula (I′) may be represented by formula (I):

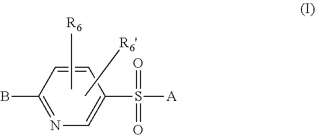

wherein A, B, R₆ and R₆′ are as defined in claim 1.

In another particular and preferred embodiment of the invention, A is a group selected from:

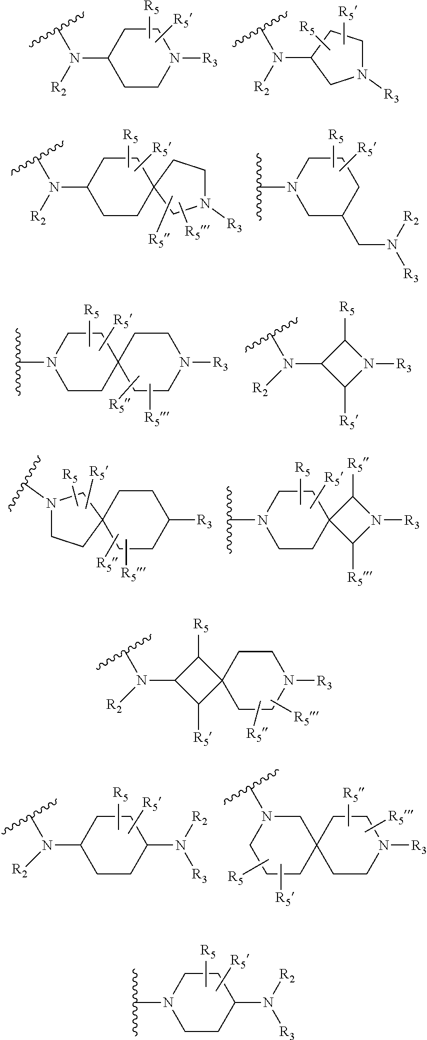

wherein R₂, R₃, R₅, R₅′, R₅″ and R₅′″ is as defined along the detailed description and claims.

In another particular and preferred embodiment of the invention, B represents a branched or unbranched C₁₋₆ alkyl radical, preferably methyl or ethyl; a tetrahydropyranyl group; a pyridinyl group optionally substituted by a C₁₋₆ haloalkyl radical; or a —NR₁R_{1′} group wherein R₁ and R₁′ independently represent a hydrogen atom; a branched or unbranched C₁₋₆ alkyl radical; or a —C(O)R where R represents a C₁₋₆ alkyl radical, with the proviso that at least one of R₁ and R₁′ is different from a hydrogen atom; or alternatively R₁ and R₁′ form together with the nitrogen atom to which they are attached one of the following structures:

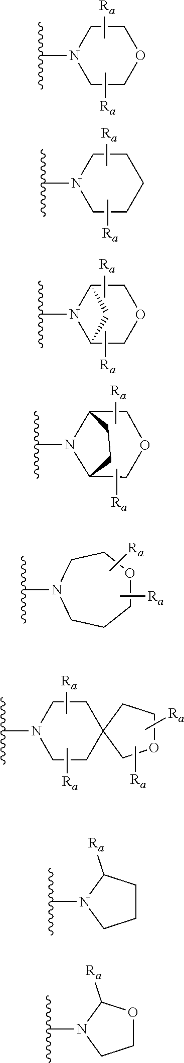

wherein each Rₐ is as defined the detailed description and claims.

In a further particular and preferred embodiment of the invention R₂ is hydrogen, methyl, ethyl or isopropyl.

An additional particular and preferred embodiment of the invention is that where R₃ is branched or unbranched C₃₋₁₀ alkyl radical optionally substituted by one or more R₃′; benzyl optionally substituted by one or more R₃′ radicals; a pyridinylmethyl optionally substituted by one or more R₃′; or a phenethyl optionally substituted by substituted by one or more R₃′

Yet another particular and preferred embodiment of the invention is that wherein R₃′ is a halogen atom, preferably F or Cl; —ON, C₁₋₆ haloalkyl, preferably trifluoromethyl; —OH or —OCH₃.

A further particular and preferred embodiment is that wherein R₅, R₅′, R₅″ and R₅′″ are independently from one another a hydrogen atom or a branched or unbranched C₁₋₆ alkyl radical, preferably a methyl group.

Similarly it is a preferred embodiment of the invention compounds wherein R₆ and R₆′ are independently from one another a hydrogen atom.

A further particular and preferred embodiment of the invention comprises a compound of general formula (I′):

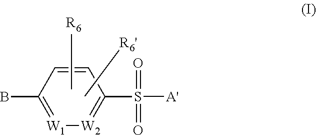

wherein:
A is one of the following moieties:

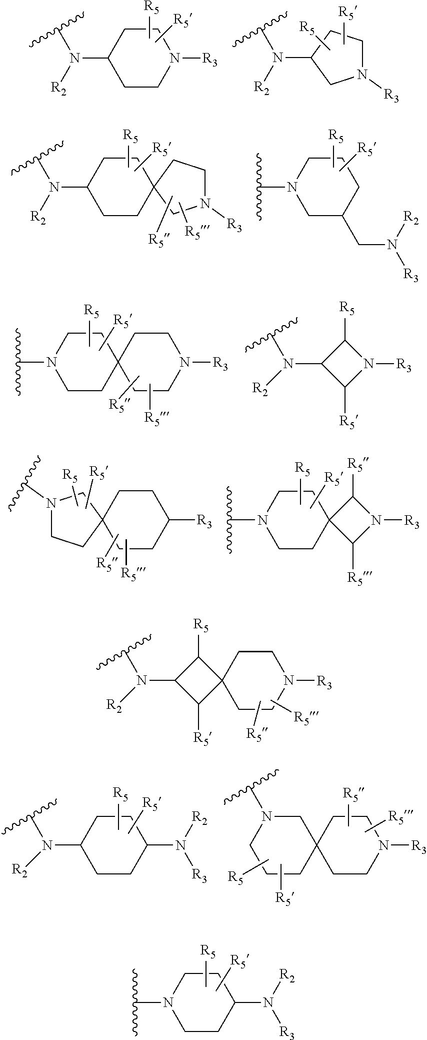

B represents a branched or unbranched C₁₋₆ alkyl radical, preferably methyl or ethyl; a tetrahydropyranyl group; a pyridinyl group optionally substituted by a C₁₋₆ haloalkyl radical; or a —NR₁R_{1′} group wherein R₁ and R₁′ independently represent a hydrogen atom; a branched or unbranched C₁₋₆alkyl radical, preferably methyl or ethyl or a —C(O)R where R represents a C₁₋₆ alkyl radical, preferably methyl or ethyl, with the proviso that at least one of R₁ and R₁′ is different from a hydrogen atom; or alternatively R₁ and R₁′ form together with the nitrogen atom to which they are attached one of the following structures:

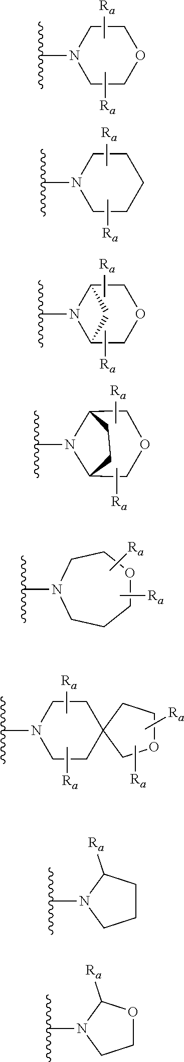

each Rₐ is independently represents a hydrogen atom; a branched or unbranched C₁₋₆ alkyl radical; a halogen atom, a ON group, a C₁₋₆ haloalkyl radical, a OH group, a carbonyl group or a C₁₋₆ alkoxy radical;
W₁ and W₂ independently represent —N— or —CH— with the proviso that one of W₁ or W₂ is —N— and the other is —CH—;
R₂ is a hydrogen atom or a branched or unbranched C₁₋₆ alkyl radical; preferably R₂ hydrogen, methyl, ethyl or isopropyl.

R₃ is a branched or unbranched C₃₋₁₀ alkyl radical; an alkylaryl radical optionally substituted by one or more R₃′ radicals, preferably benzyl or phenethyl optionally substituted by one or more R₃′ radicals; or an alkylheteroaryl radical optionally substituted by one or more R₃′ radicals; preferably a pyridinylmethyl optionally substituted by one or more R₃′;
R₃′ is a branched or unbranched C₁₋₆alkyl radical, a halogen atom, a CN group, a C₁₋₆ haloalkyl radical, a OH group or a C₁₋₆ alkoxy radical;
R₅, R₅′, R₅″ and R₅′″ are independently from one another a hydrogen atom; a branched or unbranched 1-6 alkyl radical, preferably methyl; 1-6 alkoxy radical; a halogen atom; C₁₋₆ haloalkyl group; a OH group or a CN group;
R₆ and R₆′ are independently from one another a hydrogen atom; a branched or unbranched C₁₋₆ alkyl radical; C₁₋₆ alkoxy radical; a halogen atom, preferably F; C₁₋₆ haloalkyl group; a OH group or a CN group;
r is 0, 1 or 2;
with the proviso that when R₃ is a benzyl group R_{3′} is not an a 1-6 alkoxy radical;
wherein the compound of formula (I′) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt, co-crystal or prodrug thereof, or a corresponding solvate thereof.

A still further particular and preferred embodiment of the invention comprises a compound of formula (I):

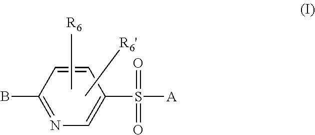

wherein:
A is one of the following moieties:

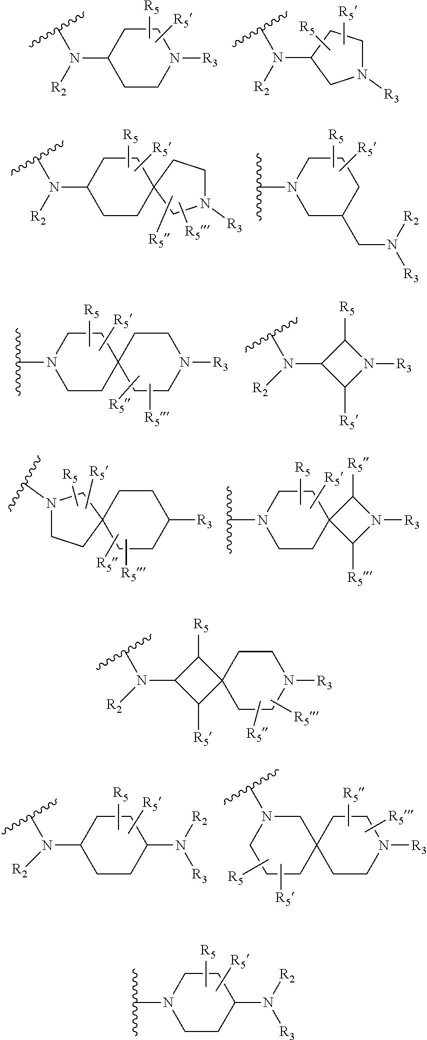

B represents methyl or ethyl; a tetrahydropyranyl group; a pyridinyl group optionally substituted by a C₁₋₆ haloalkyl, preferably trifluoromethyl; or a —NR₁R_{1′} group wherein R₁ and R₁′ independently represent a hydrogen atom; a methyl or ethyl radical or a —C(O)R where R represents a C methyl or ethyl, with the proviso that at least one of R₁ and R₁′ is different from a hydrogen atom; or alternatively R₁ and R₁′ form together with the nitrogen atom to which they are attached one of the following structures:

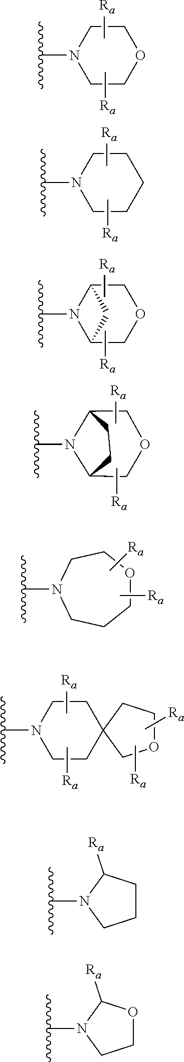

each Rₐ is independently a hydrogen atom; a branched or unbranched C₁₋₆ alkyl radical; a halogen atom, a CN group, a C₁₋₆ haloalkyl radical, a OH group; a carbonyl group or a C₁₋₆ alkoxy radical;
R₂ is a hydrogen atom or a branched or methyl, ethyl or isopropyl.
R₃ is a branched or unbranched C₃₋₁₀ alkyl radical; benzyl or phenethyl optionally substituted by one or more R₃′ radicals; or a pyridinylmethyl optionally substituted by one or more R₃′;
R₃′ is a branched or unbranched C₁₋₆alkyl radical, a halogen atom, a CN group, a C₁₋₆ haloalkyl radical, a OH group or a C₁₋₆ alkoxy radical;
R₅, R₅′, R₅″ and R₅′″ are independently from one another a hydrogen atom; a branched or unbranched C₁₋₆ alkyl radical, preferably methyl; C₁₋₆ alkoxy radical; a halogen atom; C₁₋₆ haloalkyl group; a OH group or a ON group;
R₆ and R₆′ are independently from one another a hydrogen atom; or a halogen atom preferably F;
r is 0, 1 or 2;
with the proviso that when R₃ is a benzyl group R₃ is not an a C₁₋₆ alkoxy radical;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt, co-crystal or prodrug thereof, or a corresponding solvate thereof.

A particularly preferred embodiment of the invention is represented by compounds of formula (I) having subformulas (Ia), (Ib), (Ic), (Id), (Ie), (If) or (Ig):

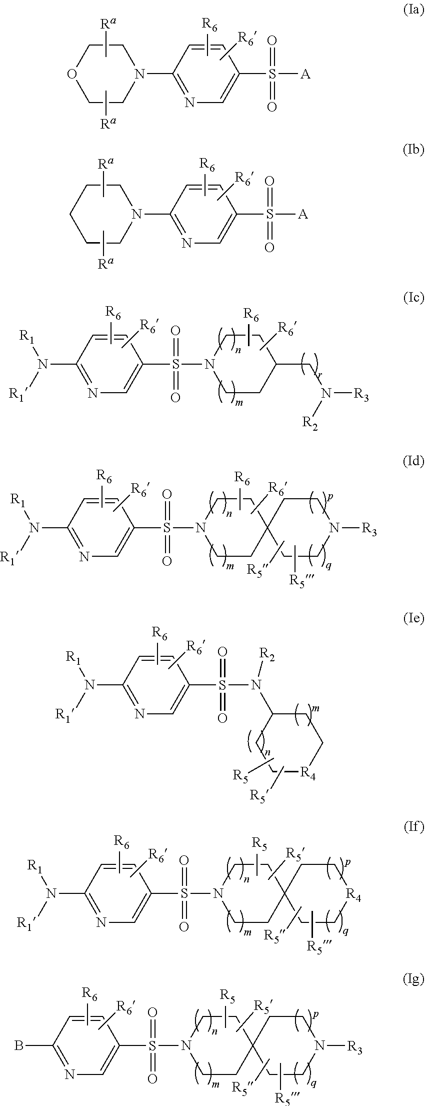

wherein A, B, Rₐ, R₁, R₁′, R₂, R₃, R₄, R₅, R₅′, R₅″, R₅′″, R₆, R₆′, n, m, p, q and r are as defined for formula (I) along the detailed description and claims.

The compounds of the present invention represented by the above described formula (I), (Ia), (Ib), (Ic), (Id) (Ie), (If) or (Ig) may include enantiomers depending on the presence of chiral centers or isomers depending on the presence of double bonds (e.g. Z, E). The single stereoisomers, enantiomers or diastereoisomers and mixtures thereof fall within the scope of the present invention.

The preferred compounds of the invention are selected from:
[1] (S)—N-(1-Benzylpyrrolidin-3-yl)-6-morpholinopyridine-3-sulfonamide;
[2] N-(1-Benzylpiperidin-4-yl)-6-morpholinopyridine-3-sulfonamide;
[3] (R)—N-(1-Benzylpyrrolidin-3-yl)-6-morpholinopyridine-3-sulfonamide;
[4] (S)—N-(1-Benzylpyrrolidin-3-yl)-N-methyl-6-morpholinopyridine-3-sulfonamide;
[5] (R)—N-(1-Benzylpyrrolidin-3-yl)-N-methyl-6-morpholinopyridine-3-sulfonamide;
[6] N-((3R,4S)-1-Benzyl-4-methylpyrrolidin-3-yl)-6-morpholinopyridine-3-sulfonamide;
[7] N-(1-(4-Fluorobenzyl)piperidin-4-yl)-6-morpholinopyridine-3-sulfonamide;
[8] N-(1-(3-Fluorobenzyl)piperidin-4-yl)-6-morpholinopyridine-3-sulfonamide;
[9] N-(1-(3-Cyanobenzyl)piperidin-4-yl)-6-morpholinopyridine-3-sulfonamide;
[10] N-(1-(4-Chlorobenzyl)piperidin-4-yl)-6-morpholinopyridine-3-sulfonamide;
[11] N-(1-(4-Cyanobenzyl)piperidin-4-yl)-6-morpholinopyridine-3-sulfonamide;
[12] 4-(5-((9-Benzyl-3,9-diazaspiro[5.5]undecan-3-yl)sulfonyl)pyridin-2-yl)morpholine;
[13] 4-(5-((2-Benzyl-2,8-diazaspiro[4.5]decan-8-yl)sulfonyl)pyridin-2-yl)morpholine;
[14] N-((3S,4R)-1-Benzyl-4-methylpyrrolidin-3-yl)-6-morpholinopyridine-3-sulfonamide;
[15] N-Methyl-1-((6-morpholinopyridin-3-yl)sulfonyl)-N-phenethylpiperidin-4-amine;
[16] N-Benzyl-N-methyl-1-((6-morpholinopyridin-3-yl)sulfonyl)piperidin-4-amine;
[17] (S)—N-Benzyl-N-methyl-1-((6-morpholinopyridin-3-yl)sulfonyl)pyrrolidin-3-amine;
[18] N-((1r,4r)-4-(Benzyl(methyl)amino)cyclohexyl)-6-morpholinopyridine-3-sulfonamide;
[19] N-((1 s,4s)-4-(Benzyl(methyl)amino)cyclohexyl)-6-morpholinopyridine-3-sulfonamide;
[20] N—((S)-1-Benzylpyrrolidin-3-yl)-6-((2R,6S)-2,6-dimethylmorpholino)pyridine-3-sulfonamide;
[21] (S)—N-(1-Benzylpyrrolidin-3-yl)-6-(dimethylamino)pyridine-3-sulfonamide;
[22] (R)—N-(1-Benzylpyrrolidin-3-yl)-6-(dimethylamino)pyridine-3-sulfonamide;
[23] N—((R)-1-Benzylpyrrolidin-3-yl)-6-((1 S,4S)-2-oxa-5-azabicyclo[2.2.1]heptan-5-yl)pyridine-3-sulfonamide;
[24] (R)—N-(1-Benzylpyrrolidin-3-yl)-5-fluoro-6-morpholinopyridine-3-sulfonamide;
[25] (S)—N-(1-Benzylpyrrolidin-3-yl)-5-fluoro-6-morpholinopyridine-3-sulfonamide;
[26] (R)—N-(1-Benzylpyrrolidin-3-yl)-6-(4-methoxypiperidin-1-yl)pyridine-3-sulfonamide;
[27] N—((R)-1-Benzylpyrrolidin-3-yl)-6-((1R,5S)-3-oxa-8-azabicyclo[3.2.1]octan-8-yl)pyridine-3-sulfonamide;
[28] N-((1r,4r)-4-((4-Fluorobenzyl)(methyl)amino)cyclohexyl)-6-morpholinopyridine-3-sulfonamide;
[29] 4-(5-((9-(3,3-Dimethylbutyl)-3,9-diazaspiro[5.5]undecan-3-yl)sulfonyl)pyridin-2-yl)morpholine;
[30] N-((1 r,4r)-4-(Benzyl(methyl)amino)cyclohexyl)-6-(dimethylamino)pyridine-3-sulfonamide;
[31] N-((1 r,4r)-4-(Benzyl(methyl)amino)cyclohexyl)-6-(1-oxa-8-azaspiro[4.5]decan-8-yl)pyridine-3-sulfonamide;
[32] N-((1 r,4r)-4-(Benzyl(methyl)amino)cyclohexyl)-6-(1,4-oxazepan-4-yl)pyridine-3-sulfonamide;
[33] (R)-1-(6-Methoxypyridin-3-yl)-N-((1-((6-morpholinopyridin-3-yl)sulfonyl)piperidin-3-yl)methyl)methanamine;
[34] (S)-1-(6-Methoxypyridin-3-yl)-N-((1-((6-morpholinopyridin-3-yl)sulfonyl)piperidin-3-yl)methyl)methanamine;
[35] (S)—N-(1-(3-Fluorobenzyl)pyrrolidin-3-yl)-6-morpholinopyridine-3-sulfonamide;
[36] (R)—N-(1-(4-Fluorobenzyl)pyrrolidin-3-yl)-6-morpholinopyridine-3-sulfonamide;
[37] (R)—N-(1-(4-Cyanobenzyl)pyrrolidin-3-yl)-6-morpholinopyridine-3-sulfonamide;
[38] (R)—N-(1-(3-Fluorobenzyl)pyrrolidin-3-yl)-6-morpholinopyridine-3-sulfonamide;
[39] (R)—N-(1-(3-Fluorobenzyl)pyrrolidin-3-yl)-6-morpholinopyridine-3-sulfonamide;
[40] (R)—N-(1-(3-Cyanobenzyl)pyrrolidin-3-yl)-6-morpholinopyridine-3-sulfonamide;
[41] (S)—N-(1-(4-Fluorobenzyl)pyrrolidin-3-yl)-6-morpholinopyridine-3-sulfonamide;
[42] (S)—N-(1-(3-Cyanobenzyl)pyrrolidin-3-yl)-6-morpholinopyridine-3-sulfonamide;
[43] N-(1-Benzylazetidin-3-yl)-6-morpholinopyridine-3-sulfonamide;
[44] (R)-6-Morpholino-N-(1-((6-(trifluoromethyl)pyridin-3-yl)methyl)pyrrolidin-3-yl)pyridine-3-sulfonamide;
[45] 4-(5-((8-Benzyl-2,8-diazaspiro[4.5]decan-2-yl)sulfonyl)pyridin-2-yl)morpholine;
[46] N-(7-Benzyl-7-azaspiro[3.5]nonan-2-yl)-6-morpholinopyridine-3-sulfonamide;
[47] 4-(5-((2-Benzyl-2,7-diazaspiro[3.5]nonan-7-yl)sulfonyl)pyridin-2-yl)morpholine;
[48] 3-((9-((6-Morpholinopyridin-3-yl)sulfonyl)-3,9-diazaspiro[5.5]undecan-3-yl)methyl)benzonitrile;
[49] (R)—N-(1-(4-Cyanobenzyl)pyrrolidin-3-yl)-N-isopropyl-6-morpholinopyridine-3-sulfonamide;
[50] (S)—N-(1-(4-Cyanobenzyl) pyrrolidin-3-yl)-N-isopropyl-6-morpholinopyridine-3-sulfonamide;
[51] N-((1r,4r)-4-(Benzylamino)cyclohexyl)-6-morpholinopyridine-3-sulfonamide;
[52] N-((1 r,4r)-4-((3-Cyano-4-fluorobenzyl)amino)cyclohexyl)-6-morpholinopyridine-3-sulfonamide;
[53] N-((1 r,4r)-4-((2-Fluorobenzyl)amino)cyclohexyl)-6-morpholinopyridine-3-sulfonamide;
[54] N-((1 r,4r)-4-((3-Fluorobenzyl)amino)cyclohexyl)-6-morpholinopyridine-3-sulfonamide;
[55] 6-Morpholino-N-((1r,4r)-4-((pyridin-4-ylmethyl)amino)cyclohexyl)pyridine-3-sulfonamide;
[56] N-((1r,4r)-4-((2-Fluorobenzyl)(methyl)amino)cyclohexyl)-6-morpholinopyridine-3-sulfonamide;
[57] N-((1r,4r)-4-((3-Fluorobenzyl)(methyl)amino)cyclohexyl)-6-morpholinopyridine-3-sulfonamide;
[58] N-((1 r,4r)-4-((3-Cyano-4-Fluorobenzyl)(methyl)amino)cyclohexyl)-6-morpholinopyridine-3-sulfonamide;
[59] N-((1 r,4r)-4-(Methyl(pyridin-3-ylmethyl)amino)cyclohexyl)-6-morpholinopyridine-3-sulfonamide;
[60] N-((1 r,4r)-4-(Methyl(pyridin-4-ylmethyl)amino)cyclohexyl)-6-morpholinopyridine-3-sulfonamide;
[61] 4-(5-((9-Benzyl-2,9-diazaspiro[5.5]undecan-2-yl)sulfonyl)pyridin-2-yl)morpholine;
[62] 2-Fluoro-4-((2-((6-morpholinopyridin-3-yl)sulfonyl)-2,9-diazaspiro[5.5]undecan-9-yl)methyl)benzonitrile;
[63] 4-(5-((9-(Pyridin-4-ylmethyl)-2,9-diazaspiro[5.5]undecan-2-yl)sulfonyl)pyridin-2-yl)morpholine;
[64] (R)-5-((((1-((6-Morpholinopyridin-3-yl)sulfonyl)piperidin-3-yl)methyl)amino)methyl)pyridin-2-ol;
[65] (S)-5-((((1-((6-Morpholinopyridin-3-yl)sulfonyl)piperidin-3-yl)methyl)amino)methyl)pyridin-2-ol;
[66] 3-(3,3-Dimethylbutyl)-9-((6-(trifluoromethyl)pyridin-3-yl)sulfonyl)-3,9-diazaspiro[5.5]undecane;
[67] 8-(3,3-Dimethylbutyl)-2-((6-(trifluoromethyl) pyridin-3-yl)sulfonyl)-2,8-diazaspiro[4.5]decane;
[68] 8-(5-((9-(3,3-Dimethylbutyl)-3,9-diazaspiro[5.5]undecan-3-yl)sulfonyl)pyridin-2-yl)-2-oxa-8-azaspiro[4.5]decane;
[69] 4-(5-((9-(3,3-Dimethylbutyl)-3,9-diazaspiro[5.5]undecan-3-yl)sulfonyl)pyridin-2-yl)thiomorpholine 1,1-dioxide;
[70] 3-((6-(4,4-Difluoropiperidin-1-yl)pyridin-3-yl)sulfonyl)-9-(3,3-dimethylbutyl)-3,9-diazaspiro[5.5]undecane;
[71] 8-(5-((9-(3,3-Dimethylbutyl)-3,9-diazaspiro[5.5]undecan-3-yl)sulfonyl)pyridin-2-yl)-1-oxa-8-azaspiro[4.5]decane;
[72] 7-(5-((9-(3,3-Dimethylbutyl)-3,9-diazaspiro[5.5]undecan-3-yl)sulfonyl)pyridin-2-yl)-2-oxa-7-azaspiro[3.5]nonane;
[73] 3-((6-(4,4-Difluoropiperidin-1-yl)pyridin-3-yl)sulfonyl)-9-isopentyl-3,9-diazaspiro[5.5]undecane;
[74] 3-Denzyl-9-((6-(4,4-difluoropiperidin-1-yl)pyridin-3-yl)sulfonyl)-3,9-diazaspiro[5.5]undecane;
[75] 4-(5-((2-(3,3-Dimethylbutyl)-2,9-diazaspiro[5.5]undecan-9-yl)sulfonyl)pyridin-2-yl)morpholine;
[76] 9-((6-(4,4-Difluoropiperidin-1-yl)pyridin-3-yl)sulfonyl)-2-(3,3-dimethylbutyl)-2,9-diazaspiro[5.5]undecane;
[77] 9-((6-(4,4-Difluoropiperidin-1-yl)pyridin-3-yl)sulfonyl)-2-isopentyl-2,9-diazaspiro[5.5]undecane;
[78] 8-(5-((2-(3,3-Dimethylbutyl)-2,9-diazaspiro[5.5]undecan-9-yl)sulfonyl)pyridin-2-yl)-1-oxa-8-azaspiro[4.5]decane;
[79] 4-(5-((8-(3,3-Dimethylbutyl)-2,8-diazaspiro[4.5]decan-2-yl)sulfonyl)pyridin-2-yl)morpholine;
[80] 2-((6-(4,4-Difluoropiperidin-1-yl)pyridin-3-yl)sulfonyl)-8-(3,3-dimethylbutyl)-2,8-diazaspiro[4.5]decane;
[81] 8-(5-((8-(3,3-Dimethylbutyl)-2,8-diazaspiro[4.5]decan-2-yl)sulfonyl)pyridin-2-yl)-1-oxa-8-azaspiro[4.5]decane;
[82] 8-Benzyl-2-((6-(4,4-difluoropiperidin-1-yl)pyridin-3-yl)sulfonyl)-2,8-diazaspiro[4.5]decane;
[83] 2-((6-(4,4-Difluoropiperidin-1-yl)pyridin-3-yl)sulfonyl)-8-((tetrahydro-2H-pyran-4-yl)methyl)-2,8-diazaspiro[4.5]decane;
[84] 9-((6-(4,4-Difluoropiperidin-1-yl)pyridin-3-yl)sulfonyl)-2-((tetrahydro-2H-pyran-4-yl)methyl)-2,9-diazaspiro[5.5]undecane;
[85] 9-((6-(4,4-Difluoropiperidin-1-yl)pyridin-3-yl)sulfonyl)-2-neopentyl-2,9-diazaspiro[5.5]undecane;
[86] N-Benzyl-1-((6-(4,4-difluoropiperidin-1-yl)pyridin-3-yl)sulfonyl)-N-methylpiperidin-4-amine;
[87] 1-((6-(1-Oxa-8-azaspiro[4.5]decan-8-yl)pyridin-3-yl)sulfonyl)-N-benzyl-N-methylpiperidin-4-amine;
[88] 4-(5-((9-(3,3-Dimethylbutyl)-2,9-diazaspiro[5.5]undecan-2-yl)sulfonyl)pyridin-2-yl)morpholine;
[89] N-((1 r,4r)-4-((3,3-Dimethylbutyl)(methyl)amino)cyclohexyl)-6-morpholinopyridine-3-sulfonamide;
[90] 3-(2-Isopropoxyethyl)-9-((6-(trifluoromethyl) pyridin-3-yl)sulfonyl)-3,9-diazaspiro[5.5]undecane;
[91] 3-Isobutyl-9-((6-(trifluoromethyl) pyridin-3-yl)sulfonyl)-3,9-diazaspiro[5.5]undecane;
[92] 9-(3,3-Dimethylbutyl)-2-((6-(trifluoromethyl)pyridin-3-yl)sulfonyl)-2,9-diazaspiro[5.5]undecane;
[93] 3-Isopentyl-9-((6-(trifluoromethyl)pyridin-3-yl)sulfonyl)-3,9-diazaspiro[5.5]undecane;
[94] 5-((9-(3,3-Dimethylbutyl)-2,9-diazaspiro[5.5]undecan-2-yl)sulfonyl)-N-methylpyridin-2-amine;
[95] 8-(5-((9-(3,3-Dimethylbutyl)-2,9-diazaspiro[5.5]undecan-2-yl)sulfonyl)pyridin-2-yl)-2-oxa-8-azaspiro[4.5]decane;
[96] 2-((6-(4,4-Difluoropiperidin-1-yl)pyridin-3-yl)sulfonyl)-9-(3,3-dimethylbutyl)-2,9-diazaspiro[5.5]undecane;
[97] 5-((9-(3,3-Dimethylbutyl)-2,9-diazaspiro[5.5]undecan-2-yl)sulfonyl)-N,N-dimethylpyridin-2-amine;
[98] 8-(5-((9-(3,3-Dimethylbutyl)-2,9-diazaspiro[5.5]undecan-2-yl)sulfonyl)pyridin-2-yl)-1-oxa-8-azaspiro[4.5]decane;
[99] 3-((6-(4,4-Difluoropiperidin-1-yl)pyridin-3-yl)sulfonyl)-9-neopentyl-3,9-diazaspiro[5.5]undecane;
[100] 1-((6-(4,4-Difluoropiperidin-1-yl)pyridin-3-yl)sulfonyl)-N-methyl-N-neopentylpiperidin-4-amine;
[101] 2-((6-(4,4-Difluoropiperidin-1-yl)pyridin-3-yl)sulfonyl)-9-((tetrahydro-2H-pyran-4-yl)methyl)-2,9-diazaspiro[5.5]undecane;
[102] N-(5-((9-(3,3-Dimethylbutyl)-2,9-diazaspiro[5.5]undecan-2-yl)sulfonyl)pyridin-2-yl)-N-methylacetamide;
[103] N-(5-((9-(3,3-Dimethylbutyl)-3,9-diazaspiro[5.5]undecan-3-yl)sulfonyl)pyridin-2-yl)-N-methylacetamide;
[104] N-(5-((9-(3,3-Dimethylbutyl)-2,9-diazaspiro[5.5]undecan-2-yl)sulfonyl)pyridin-2-yl)-N-methylpropionamide;
[105] N-(5-((9-(3,3-Dimethylbutyl)-3,9-diazaspiro[5.5]undecan-3-yl)sulfonyl)pyridin-2-yl)-N-methylpropionamide;
[106] N-(5-((9-(3,3-Dimethylbutyl)-2,9-diazaspiro[5.5]undecan-2-yl)sulfonyl)pyridin-2-yl)propionamide;
[107] N-(5-((9-(3,3-Dimethylbutyl)-2,9-diazaspiro[5.5]undecan-2-yl)sulfonyl)pyridin-2-yl)acetamide;
[108] 9-(3,3-Dimethylbutyl)-2-((6-methylpyridin-3-yl)sulfonyl)-2,9-diazaspiro[5.5]undecane;
[109] 3-(3,3-Dimethylbutyl)-9-((6-ethylpyridin-3-yl)sulfonyl)-3,9-diazaspiro[5.5]undecane;
[110] 3-(3,3-Dimethylbutyl)-9-((6-methylpyridin-3-yl)sulfonyl)-3,9-diazaspiro[5.5]undecane;
[111] 9-(3,3-Dimethylbutyl)-2-((6-ethylpyridin-3-yl)sulfonyl)-2,9-diazaspiro[5.5]undecane;
[112] 2-([2,4′-Bipyridin]-5-ylsulfonyl)-9-(3,3-dimethylbutyl)-2,9-diazaspiro[5.5]undecane;
[113] 3-([2,4′-Bipyridin]-5-ylsulfonyl)-9-(3,3-dimethylbutyl)-3,9-diazaspiro[5.5]undecane;
[114] 2-([2,4′-Bipyridin]-5-ylsulfonyl)-8-(3,3-dimethylbutyl)-2,8-diazaspiro[4.5]decane;
[115] 9-([2,4′-Bipyridin]-5-ylsulfonyl)-2-(3,3-dimethylbutyl)-2,9-diazaspiro[5.5]undecane;
[116] 9-(3,3-Dimethylbutyl)-2-((6-(tetrahydro-2H-pyran-4-yl)pyridin-3-yl)sulfonyl)-2,9-diazaspiro[5.5]undecane;
[117] 3-(3,3-Dimethylbutyl)-9-((6-(tetrahydro-2H-pyran-4-yl)pyridin-3-yl)sulfonyl)-3,9-diazaspiro[5.5]undecane;
[118] 4-(5-((9-(3,3-Dimethylbutyl)-2,9-diazaspiro[5.5]undecan-2-yl)sulfonyl)pyridin-2-yl)thiomorpholine 1,1-dioxide;
[119] N-((1r,4r)-4-((4-Fluorobenzyl)(methyl)amino)cyclohexyl)-5-morpholinopyridine-2-sulfonamide;
[120] 4-(5-((9-(3,3-Dimethylbutyl)-2,9-diazaspiro[5.5]undecan-2-yl)sulfonyl)pyridin-2-yl)morpholin-3-one;
[121] 1-(5-((9-(3,3-Dimethylbutyl)-2,9-diazaspiro[5.5]undecan-2-yl)sulfonyl)pyridin-2-yl)pyrrolidin-2-one;
[122] 1-(5-((9-(3,3-Dimethylbutyl)-2,9-diazaspiro[5.5]undecan-2-yl)sulfonyl)pyridin-2-yl)piperidin-2-one;
[123] 3-(5-((9-(3,3-Dimethylbutyl)-2,9-diazaspiro[5.5]undecan-2-yl)sulfonyl)pyridin-2-yl)oxazolidin-2-one;
[124] 3-(5-((4-(Methyl(neopentyl)amino)piperidin-1-yl)sulfonyl)pyridin-2-yl)oxazolidin-2-one;
[125] 3-Fluoro-4-((2-((6-(2-oxooxazolidin-3-yl)pyridin-3-yl)sulfonyl)-2,9-diazaspiro[5.5]undecan-9-yl)methyl)benzonitrile;
[126] 1-(5-((9-((Tetrahydro-2H-pyran-4-yl)methyl)-2,9-diazaspiro[5.5]undecan-2-yl)sulfonyl)pyridin-2-yl)pyrrolidin-2-one;
[127] 3-(6-((9-((Tetrahydro-2H-pyran-4-yl)methyl)-2,9-diazaspiro[5.5]undecan-2-yl)sulfonyl)pyridin-3-yl)oxazolidin-2-one;
[128] 3-(5-((9-((Tetrahydro-2H-pyran-4-yl)methyl)-3,9-diazaspiro[5.5]undecan-3-yl)sulfonyl)pyridin-2-yl)oxazolidin-2-one;
[129] 3-(5-((4-(Methyl((tetrahydro-2H-pyran-4-yl)methyl)amino)piperidin-1-yl)sulfonyl)pyridin-2-yl)oxazolidin-2-one;
[130] 9-Benzyl-2-((6-(4,4-difluoropiperidin-1-yl)pyridin-3-yl)sulfonyl)-2,9-diazaspiro[5.5]undecane;
[131] 4-(5-((9-(3,3-Dimethylbutyl)-3,9-diazaspiro[5.5]undecan-3-yl)sulfonyl)pyridin-2-yl)morpholin-3-one;
[132] 1-(5-((9-(3,3-Dimethylbutyl)-3,9-diazaspiro[5.5]undecan-3-yl)sulfonyl)pyridin-2-yl)piperidin-2-one;
[133] 1-(5-((9-(3,3-Dimethylbutyl)-3,9-diazaspiro[5.5]undecan-3-yl)sulfonyl)pyridin-2-yl)pyrrolidin-2-one;
[134] 3-(5-((9-(3,3-Dimethylbutyl)-3,9-diazaspiro[5.5]undecan-3-yl)sulfonyl)pyridin-2-yl)oxazolidin-2-one;
[135] 3-(5-((9-Benzyl-3,9-diazaspiro[5.5]undecan-3-yl)sulfonyl)pyridin-2-yl)oxazolidin-2-one;
[136] 1-(5-((2-(3,3-Dimethylbutyl)-2,9-diazaspiro[5.5]undecan-9-yl)sulfonyl)pyridin-2-yl)pyrrolidin-2-one;
[137] 3-(5-((2-(3,3-Dimethylbutyl)-2,9-diazaspiro[5.5]undecan-9-yl)sulfonyl)pyridin-2-yl)oxazolidin-2-one;
[138] 3-(5-((8-(3,3-Dimethylbutyl)-2,8-diazaspiro[4.5]decan-2-yl)sulfonyl)pyridin-2-yl)oxazolidin-2-one;
[139] 1-(5-((8-(3,3-Dimethylbutyl)-2,8-diazaspiro[4.5]decan-2-yl)sulfonyl)pyridin-2-yl)pyrrolidin-2-one;
[140] 3-(5-((9-(3,3-Dimethylbutyl)-3,9-diazaspiro[5.5]undecan-3-yl)sulfonyl)-3-fluoropyridin-2-yl)oxazolidin-2-one;
[141] 1-(5-((9-(3,3-Dimethylbutyl)-3,9-diazaspiro[5.5]undecan-3-yl)sulfonyl)-3-fluoropyridin-2-yl)pyrrolidin-2-one;
[142] 3-(5-((4-(Benzyl(methyl)amino)piperidin-1-yl)sulfonyl)pyridin-2-yl)oxazolidin-2-one;
[143] 3-(5-((9-Neopentyl-3,9-diazaspiro[5.5]undecan-3-yl)sulfonyl)pyridin-2-yl)oxazolidin-2-one;
[144] 3-(5-((8-Benzyl-2,8-diazaspiro[4.5]decan-2-yl)sulfonyl)pyridin-2-yl)oxazolidin-2-one;
[145] 3-(5-((2-(2-Fluorobenzyl)-2,9-diazaspiro[5.5]undecan-9-yl)sulfonyl)pyridin-2-yl)oxazolidin-2-one;
[146] 3-(5-((2-(2,5-Difluorobenzyl)-2,9-diazaspiro[5.5]undecan-9-yl)sulfonyl)pyridin-2-yl)oxazolidin-2-one;
[147] 3-(5-((2-Benzyl-2,9-diazaspiro[5.5]undecan-9-yl)sulfonyl)pyridin-2-yl)oxazolidin-2-one;
[148] 1-(5-((4-(Benzyl(methyl)amino)piperidin-1-yl)sulfonyl)pyridin-2-yl)pyrrolidin-2-one;
[149] 3-(5-((2-((Tetrahydro-2H-pyran-4-yl)methyl)-2,9-diazaspiro[5.5]undecan-9-yl)sulfonyl)pyridin-2-yl)oxazolidin-2-one;
[150] 3-(5-((2-Neopentyl-2,9-diazaspiro[5.5]undecan-9-yl)sulfonyl)pyridin-2-yl)oxazolidin-2-one;
[151] 2-Fluoro-4-((9-((6-(2-oxooxazolidin-3-yl)pyridin-3-yl)sulfonyl)-2,9-diazaspiro[5.5]undecan-2-yl)methyl)benzonitrile;
[152] 2-Fluoro-5-((9-((6-(2-oxooxazolidin-3-yl)pyridin-3-yl)sulfonyl)-2,9-diazaspiro[5.5]undecan-2-yl)methyl)benzonitrile;
[153] 3-(5-((8-((Tetrahydro-2H-pyran-4-yl)methyl)-2,8-diazaspiro[4.5]decan-2-yl)sulfonyl)pyridin-2-yl)oxazolidin-2-one;
[154] 3-(6-((2-(3,3-Dimethylbutyl)-2,9-diazaspiro[5.5]undecan-9-yl)sulfonyl)pyridin-3-yl)oxazolidin-2-one;
[155] 3-(5-((9-Benzyl-2,9-diazaspiro[5.5]undecan-2-yl)sulfonyl)pyridin-2-yl)oxazolidin-2-one;
[156] 3-(5-((9-(2,5-Difluorobenzyl)-3,9-diazaspiro[5.5]undecan-3-yl)sulfonyl)pyridin-2-yl)oxazolidin-2-one;
[157] 2-Fluoro-5-((9-((6-(2-oxooxazolidin-3-yl)pyridin-3-yl)sulfonyl)-3,9-diazaspiro[5.5]undecan-3-yl)methyl)benzonitrile;
[158] 3-(5-((9-((Tetrahydro-2H-pyran-4-yl)methyl)-2,9-diazaspiro[5.5]undecan-2-yl)sulfonyl)pyridin-2-yl)oxazolidin-2-one;
[159] 3-(5-((9-(2-Fluorobenzyl)-2,9-diazaspiro[5.5]undecan-2-yl)sulfonyl)pyridin-2-yl)oxazolidin-2-one;
[160] 3-(5-((9-(2,5-Difluorobenzyl)-2,9-diazaspiro[5.5]undecan-2-yl)sulfonyl)pyridin-2-yl)oxazolidin-2-one;
[161] 2-Fluoro-5-((2-((6-(2-oxooxazolidin-3-yl)pyridin-3-yl)sulfonyl)-2,9-diazaspiro[5.5]undecan-9-yl)methyl)benzonitrile;
[162] 3-(5-((8-(2,5-Difluorobenzyl)-2,8-diazaspiro[4.5]decan-2-yl)sulfonyl)pyridin-2-yl)oxazolidin-2-one;
[163] 3-(5-((8-(2-Fluorobenzyl)-2,8-diazaspiro[4.5]decan-2-yl)sulfonyl)pyridin-2-yl)oxazolidin-2-one;
[164] 2-Fluoro-4-((2-((6-(2-oxooxazolidin-3-yl)pyridin-3-yl)sulfonyl)-2,8-diazaspiro[4.5]decan-8-yl)methyl)benzonitrile;
[165] 2-Fluoro-5-((2-((6-(2-oxooxazolidin-3-yl)pyridin-3-yl)sulfonyl)-2,8-diazaspiro[4.5]decan-8-yl)methyl)benzonitrile;
[166] 3-(5-((9-(2-Fluorobenzyl)-3,9-diazaspiro[5.5]undecan-3-yl)sulfonyl)pyridin-2-yl)oxazolidin-2-one;
[167] N-((1 r,4r)-4-(Benzyl(methyl)amino)cyclohexyl)-6-(2-oxooxazolidin-3-yl)pyridine-3-sulfonamide;
[168] 1-((6-(4,4-Difluoropiperidin-1-yl)pyridin-3-yl)sulfonyl)-N-(3,3-dimethylbutyl)-N-methylpiperidin-4-amine;
[169] 1-((6-(4,4-Difluoropiperidin-1-yl)pyridin-3-yl)sulfonyl)-N-methyl-N-((tetrahydro-2H-pyran-4-yl)methyl)piperidin-4-amine;
[170] N-Benzyl-1-((6-(4,4-difluoropiperidin-1-yl)pyridin-3-yl)sulfonyl)-N-methylpiperidin-4-amine;
4-(((1-((6-(4,4-Difluoropiperidin-1-yl)pyridin-3-yl)sulfonyl)piperidin-4-yl)(methyl)amino)methyl)-3-fluorobenzonitrile;
[172] 1-((6-(4,4-Difluoropiperidin-1-yl)pyridin-3-yl)sulfonyl)-N-(2-fluorobenzyl)-N-methylpiperidin-4-amine;
[173] 4-(((1-((6-(4,4-Difluoropiperidin-1-yl)pyridin-3-yl)sulfonyl)piperidin-4-yl)(methyl)amino)methyl)-2-fluorobenzonitrile;
[174] N-(2,5-Difluorobenzyl)-1-((6-(4,4-difluoropiperidin-1-yl)pyridin-3-yl)sulfonyl)-N-methylpiperidin-4-amine;
[175] N-((1 r,4r)-4-(Benzyl(methyl)amino)cyclohexyl)-6-(4,4-difluoropiperidin-1-yl)pyridine-3-sulfonamide;
[176] N-((1 r,4r)-4-(Benzyl(methyl)amino)cyclohexyl)-6-(1-oxa-8-azaspiro[4.5]decan-8-yl)pyridine-3-sulfonamide;
[177] N-((1 r,4r)-4-(Methyl((tetrahydro-2H-pyran-4-yl)methyl)amino)cyclohexyl)-6-(1-oxa-8-azaspiro[4.5]decan-8-yl)pyridine-3-sulfonamide;
[178] N-((1 r,4r)-4-((2-Fluorobenzyl)(methyl)amino)cyclohexyl)-6-(1-oxa-8-azaspiro[4.5]decan-8-yl)pyridine-3-sulfonamide;
[179] N-((1 r,4r)-4-((2,5-Difluorobenzyl)(methyl)amino)cyclohexyl)-6-(2-oxa-8-azaspiro[4.5]decan-8-yl)pyridine-3-sulfonamide;
[180] N-((1 r,4r)-4-((3,4-Difluorobenzyl)(methyl)amino)cyclohexyl)-6-(2-oxa-8-azaspiro[4.5]decan-8-yl)pyridine-3-sulfonamide;
[181] N-((1 r,4r)-4-((3,3-Dimethylbutyl)(methyl)amino)cyclohexyl)-6-(2-oxa-8-azaspiro[4.5]decan-8-yl)pyridine-3-sulfonamide;
[182] N-((1 r,4r)-4-(Methyl(neopentyl)amino)cyclohexyl)-6-(2-oxa-8-azaspiro[4.5]decan-8-yl)pyridine-3-sulfonamide and
[183] N-((1 r,4r)-4-(Methyl((tetrahydro-2H-pyran-4-yl)methyl)amino)cyclohexyl)-6-(2-oxa-8-azaspiro[4.5]decan-8-yl)pyridine-3-sulfonamide.
or a pharmaceutical acceptable salt, stereoisomer, co-crystal, prod rug or solvate thereof.

In another aspect, the invention refers to the process for obtaining the compounds of general formula (I′). A general procedure for obtaining all the compounds of the invention has been developed, and the synthetic routes for preparing them as well as their intermediate compounds will be explained below.

The obtained reaction products may, if desired, be purified by conventional methods, such as crystallization and chromatography. Where the process described below for the preparation of compounds of the invention give rise to mixtures of stereoisomers, these isomers may be separated by conventional techniques such as preparative chromatography. If there are chiral centers the compounds may be prepared in racemic form, or individual enantiomers may be prepared either by enantiospecific synthesis or by resolution.

A process is described for the preparation of a compound of general formula (I′):

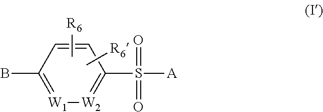

which comprises reacting a compound of formula (II):

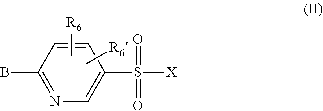

with a compound of formula (III):

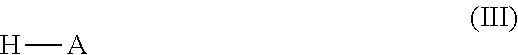

wherein A, B, W₁, W₂, R₁, R₁′, R₆ and R₆′ are as defined along the detailed description and the claims, and X represents a suitable leaving group, preferably a halogen atom.

The compounds of formula (I′) can be prepared in a one-step process by treating a sulfonyl halide of formula (II) with an amine of formula (III), in a suitable solvent, such as pyridine, dichloromethane or tetrahydrofuran; optionally, in the presence of a base such as triethylamine or N,N-diisopropylethylamine; and at a suitable temperature, preferably comprised between room temperature and the reflux temperature. Alternatively, the reactions can be carried out in a microwave reactor.

In scheme 1 below, the synthetic routes for producing compounds of formula (I′) are summarized wherein A, B, W₁, W₂, Rₐ, R₁, R₁′, R₂, R₃, R₄, R₅, R₅′, R₅″, R₅′″, R₆, R₆′, n, m, p, q and r are as defined for formula (I′) along the detailed description and claims, X represents a suitable leaving group preferably halogen, and preferably chlorine, Y represents halogen or triflate and PG represents a suitable protecting group, preferably Boc.

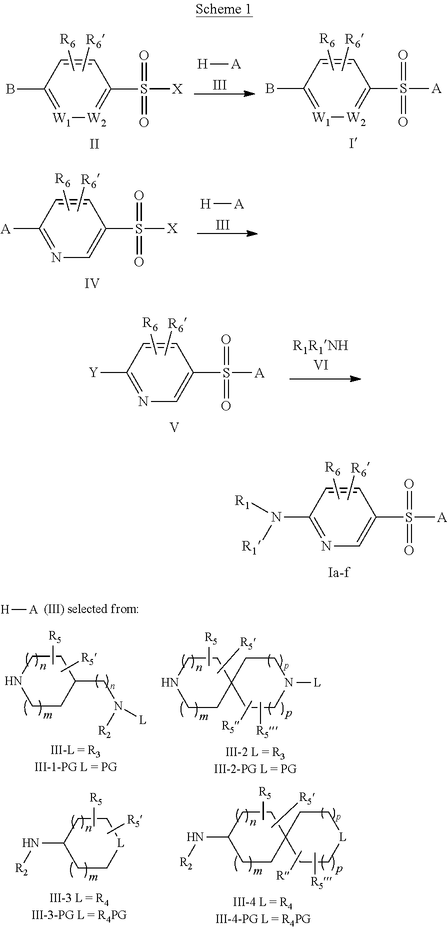

As shown in scheme 1, the compounds of formula (Ia-f) can alternatively, be prepared by introducing the group NR₁R_{1′} at a later stage, from a precursor of formula (V) and a suitable compound of formula (VI). The reaction can be carried out under nucleophilic aromatic substitution conditions, using a suitable solvent such as ethanol, N,N-dimethylacetamide (DMA), N,N-dimethylformamide (DMF) or dimethylsulfoxide (DMSO), in the presence of a suitable base such as triethylamine, N,N-diisopropylethylamine, sodium hydride or potassium tert-butoxide, at a suitable temperature, preferably heating, either thermally or irradiating in a microwave reactor. Alternatively, the reaction can be performed using a palladium catalyst, such as Pd(OAc)₂ or Pd₂(dba)₃ in a suitable solvent, such as dioxane, in the presence of a base such as K₃PO₄ or Cs₂CO₃, optionally in the presence of a suitable phosphine, such as XantPhos or DavePhos, and at a suitable temperature, preferably comprised between room temperature and the reflux temperature. Alternatively, the reactions can be carried out in a microwave reactor. The precursor compound of formula (V) can be prepared starting from a sulfonyl halide of formula (IV) following the sulfonylation conditions described for the preparation of a compound of formula I.

In another alternative approach, the group R₃ present in a compound of formula (I) can be introduced in the last step of the synthesis by reacting a compound of formula II with an N-protected precursor of a compound of formula (III), i.e. a compound of formula (III-1)-PG, (III-2)-PG, (III-3)-PG, or (III-4)-PG, to obtain an N-protected precursor of a compound of formula (I), followed by N-deprotection and final N-derivatization to incorporate the group R₃. When the protecting group is Boc, the deprotection can be conducted by adding a solution of a strong acid such as HCl, in a suitable solvent such as diethyl ether, 1,4-dioxane or methanol, or with trifluoroacetic acid in dichloromethane. The derivatization by reductive amination is carried out in the presence of a suitable ketone or aldehyde and a reductive reagent, preferably sodium triacetoxyborohydride, in a suitable solvent, such as dichloromethane, 1,2-dichloroethane or tetrahydrofuran, optionally in the presence of a base such as N,N-diisopropylethylamine or an acid such as acetic acid. The derivatization by alkylation can be carried out under standard alkylation conditions in the presence of a suitable alkylating agent, in a suitable solvent, such as acetonitrile, N,N-dimethylformamide, dimethylsulfoxide, dichloromethane, tetrahydrofuran or 1,4-dioxane, preferably in acetonitrile; in the presence of an inorganic base such as K₂CO₃ or Cs₂CO₃, or an organic base such as triethylamine or N,N-diisopropylethylamine, preferably K₂CO₃; at a suitable temperature comprised between room temperature and the reflux temperature, preferably heating. Additionally, an activating agent such as NaI can be used.

Alternatively, the compounds of formula (I′) can be prepared, as described in scheme 2, by treating a precursor compound of formula (V):
with a suitable amide, such as acetamide or propionamide, using a palladium catalyst, such as Pd₂(dba)₃ in a suitable solvent, such as dioxane, in the presence of a base such as K₃PO₄, optionally in the presence of a suitable phosphine, such as XantPhos, and at a suitable temperature, preferably at the reflux temperature. Alternatively, the reactions can be carried out in a microwave reactor.
with a suitable boronic acid of formula (VII), using a palladium catalyst, such as Pd(PPh₃)₄ or Pd(dppf)Cl₂ in a suitable solvent, such as a mixture of dioxane and water, in the presence of a base such as Na₂CO₃; and at a suitable temperature, preferably comprised between room temperature and the reflux temperature. Alternatively, the reactions can be carried out in a microwave reactor.
with a suitable alkyl zinc reagent of formula (VIII), using a nickel catalyst, such as Ni(dppp)Cl₂ in a suitable solvent, such as tetrahydrofuran and at a suitable temperature, preferably at room temperature.

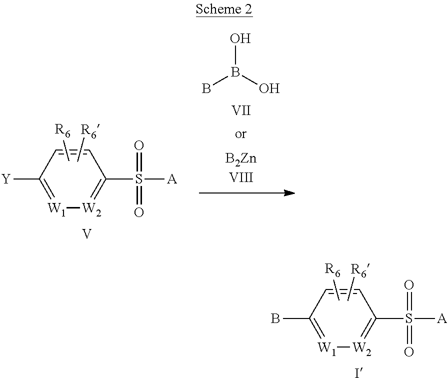

The compounds of formula (II), (III), (III-1), (III-1)-PG, (III-2), (III-2)-PG, (III-3), (III-3)-PG, (III-4), (III-4)-PG, (IV), (VII) and (VIII) are commercially available or can be synthesized following common procedures described in the literature.

In some of the processes described above, it may be necessary to protect the amino groups present in any of the compounds with suitable protecting groups, such as for example Boc (tert-butoxycarbonyl), Fmoc (fluorenylmethyloxycarbonyl), Cbz (benzyloxycarbonyl) or benzyl. The procedures for the introduction and removal of these protecting groups are well known in the art and can be found thoroughly described in the literature. As a way of example, for Boc as protecting group, the deprotection can be conducted by adding a solution of a strong acid such as HCl, in a suitable solvent such as diethyl ether, 1,4-dioxane or methanol, or with trifluoroacetic acid in dichloromethane. For Fmoc as protecting group, the deprotection is usually performed under basic media, such as for example diethylamine or piperidine in dichloromethane or N,N-dimethylformamide. When the protecting group is Cbz or benzyl, the deprotection reaction is preferably carried out by hydrogenation under hydrogen atmosphere and metal catalysis, preferably by the use of palladium or palladium hydroxide over charcoal as catalyst, in a suitable solvent such as methanol or ethanol, optionally in the presence of an acid such as acetic acid or hydrochloric acid.

Finally, a compound of formula (I′) or (I) can be obtained in enantiopure form by resolution of a racemic compound of formula (I′) or (I) or a diastereomeric mixture, either by chiral preparative HPLC or by crystallization of a diastereomeric salt or co-crystal. Alternatively, the resolution step can be carried out at a previous stage, using any suitable intermediate.

Turning to another aspect, the invention also relates to the therapeutic use of the compounds of general formula (I′) or (I). As mentioned above, compounds of general formula (I′) show a strong affinity to sigma receptors, especially to sigma-1 and/or sigma-2 receptors and can behave as agonists, antagonists, inverse agonists, partial antagonists or partial agonists thereof. Therefore, compounds of general formula (I′) or (I) are useful as medicaments.

They are suitable for the treatment and/or prophylaxis of diseases and/or disorders mediated by sigma receptors and preferably by sigma-1 and/or sigma 2 receptors. In this sense, compounds of formula (I′) or (I) are suitable for the treatment and/or prophylaxis of pain, especially neuropathic pain, inflammatory pain, and chronic pain or other pain conditions involving allodynia and/or hyperalgesia, or CNS disorder or diseases, selected from the group consisting of addiction to drugs and chemical substances including cocaine, amphetamine, ethanol and nicotine, anxiety, attention-deficit-/hyperactivity disorder (ADHD), autism spectrum disorder, catalepsy, cognition disorder, learning, memory and attention deficit, depression, encephalitis, epilepsy, headache disorder, insomnia, locked-in-syndrome, meningitis, migraine, multiple sclerosis (MS), leukodystrophies, amyotrophic lateral sclerosis (ALS), myelopathy, narcolepsy, neurodegenerative disease, traumatic brain injury, Alzheimer disease, Gaucher's disease, Huntington disease, Parkinson disease, Tourette's syndrome, psychotic condition, bipolar disorder, schizophrenia or paranoia.

The compounds of general formula (I′) or (I) are especially suited for the treatment of pain, especially neuropathic pain, inflammatory pain or other pain conditions involving allodynia and/or hyperalgesia or CNS disorder or diseases, selected from the group consisting of addiction to drugs and chemical substances including cocaine, amphetamine, ethanol and nicotine, anxiety, attention-deficit-/hyperactivity disorder (ADHD), autism spectrum disorder, catalepsy, cognition disorder, learning, memory and attention deficit, depression, encephalitis, epilepsy, headache disorder, insomnia, locked-in-syndrome, meningitis, migraine, multiple sclerosis (MS), leukodystrophies, amyotrophic lateral sclerosis (ALS), myelopathy, narcolepsy, neurodegenerative disease, traumatic brain injury, Alzheimer disease, Gaucher's disease, Huntington disease, Parkinson disease, Tourette's syndrome, psychotic condition, bipolar disorder, schizophrenia or paranoia.

Pain is defined by the International Association for the Study of Pain (IASP) as “an unpleasant sensory and emotional experience associated with actual or potential tissue damage, or described in terms of such damage” (IASP, Classification of chronic pain, 2nd Edition, IASP Press (2002), 210). Even though pain is always subjective its causes or syndromes can be classified.

In a preferred embodiment compounds of the invention are used for the treatment and/or prophylaxis of allodynia and more specifically mechanical or thermal allodynia.

In another preferred embodiment compounds of the invention are used for the treatment and/or prophylaxis of hyperalgesia.

In yet another preferred embodiment the compounds of the invention are used for the treatment and/or prophylaxis of neuropathic pain and more specifically for the treatment and/or prophylaxis of hyperpathia.

A related aspect of the invention refers to the use of compounds of general formula (I′) or (I) for the manufacture of a medicament for the treatment and/or prophylaxis of disorders and diseases mediated by sigma receptors and more preferably by sigma-1 receptors and/or sigma-2 receptors, as explained before.

Another related aspect of the invention refers to a method for the treatment and/or prophylaxis of disorders and diseases mediated by sigma receptors and more preferably by sigma-1 receptors and/or sigma-2 receptors, as explained before comprising the administration of a therapeutically effective amount of a compound of general formula (I′) or (I) to a subject in need thereof.

Another aspect of the invention is a pharmaceutical composition, which comprises at least a compound of general formula (I′) or (I) or a pharmaceutically acceptable salt, isomer, co-crystal, prodrug or solvate thereof, and at least a pharmaceutically acceptable carrier, additive, adjuvant or vehicle.

The pharmaceutical composition of the invention can be formulated as a medicament in different pharmaceutical forms comprising at least a compound binding to the sigma receptor and optionally at least one further active substance and/or optionally at least one auxiliary substance.

The auxiliary substances or additives can be selected among carriers, excipients, support materials, lubricants, fillers, solvents, diluents, colorants, flavour conditioners such as sugars, antioxidants and/or agglutinants. In the case of suppositories, this may imply waxes or fatty acid esters or preservatives, emulsifiers and/or carriers for parenteral application. The selection of these auxiliary materials and/or additives and the amounts to be used will depend on the form of application of the pharmaceutical composition.

The pharmaceutical composition in accordance with the invention can be adapted to any form of administration, be it orally or parenterally, for example pulmonarily, nasally, rectally and/or intravenously.

Preferably, the composition is suitable for oral or parenteral administration, more preferably for oral, intravenous, intraperitoneal, intramuscular, subcutaneous, intrathekal, rectal, transdermal, transmucosal or nasal administration.

The composition of the invention can be formulated for oral administration in any form preferably selected from the group consisting of tablets, dragées, capsules, pills, chewing gums, powders, drops, gels, juices, syrups, solutions and suspensions. The composition of the present invention for oral administration may also be in the form of multiparticulates, preferably microparticles, microtablets, pellets or granules, optionally compressed into a tablet, filled into a capsule or suspended in a suitable liquid. Suitable liquids are known to those skilled in the art.

Suitable preparations for parenteral applications are solutions, suspensions, reconstitutable dry preparations or sprays.

The compounds of the invention can be formulated as deposits in dissolved form or in patches, for percutaneous application.

Skin applications include ointments, gels, creams, lotions, suspensions or emulsions.

The preferred form of rectal application is by means of suppositories.

In a preferred embodiment, the pharmaceutical compositions are in oral form, either solid or liquid. Suitable dose forms for oral administration may be tablets, capsules, syrops or solutions and may contain conventional excipients known in the art such as binding agents, for example syrup, acacia, gelatin, sorbitol, tragacanth, or polyvinylpyrrolidone; fillers, for example lactose, sugar, maize starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

The solid oral compositions may be prepared by conventional methods of blending, filling or tabletting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the appropriate unit dosage form. Adequate excipients can be used, such as bulking agents, buffering agents or surfactants.

The mentioned formulations will be prepared using standard methods such as those described or referred to in the Spanish and US Pharmacopoeias and similar reference texts.

The daily dosage for humans and animals may vary depending on factors that have their basis in the respective species or other factors, such as age, sex, weight or degree of illness and so forth. The daily dosage for humans may preferably be in the range from 1 to 2000, preferably 1 to 1500, more preferably 1 to 1000 milligrams of active substance to be administered during one or several intakes per day.

The following examples are merely illustrative of certain embodiments of the invention and cannot be considered as restricting it in any way.

### EXAMPLES

The following abbreviations are used in the intermediates and examples:
ACN: acetonitrile
AcOH: acetic acid
aq.: aqueous
CH: cyclohexane
DavePhos:2-dicyclohexylphosphino-2′-(N,N-dimethylamino)biphenyl
DOE: dichloroethane
DCM: dichloromethane
Dba: dibenzylideneacetone
DIPEA: N,N-diisopropylethylamine
Dppp: 1,3-bis(diphenylphosphino)propane
Dppf: 1,1′-ferrocenediyl-bis(diphenylphosphine)
EtOAc: ethyl acetate
EtOH: ethanol
EX: example
h: hour/s
HPLC: high performance liquid chromatography
MeOH: methanol
MS: mass spectrometry
min: minutes
Quant: quantitative
Rt.: retention time
rt: room temperature
Sat: saturated
Sol.: solution
TEA: triethylamine
TFA: trifluoroacetic acid
THF: tetrahydrofuran
XantPhos: 4,5-bis(diphenylphosphino)-9,9-dimethylxanthene
wt: weight

The following methods were used to determine the HPLC-MS spectra:

### Method A

Column: KINETEX EVO 50×4.6 mm, 2.6 μm; flow rate 1.5 mL/min; temperature: 40° C.; A: NH₄HCO₃ pH 8, B: ACN; gradient: 0.5 min 95% A, 95% A to 100% B in 6.5 min; isocratic 2 min 100% B. Sample dissolved approx. 1 mg/mL in NH₄HCO₃ pH 8/ACN.

### Method B

Column ZORBAX Extend-C18 RRHD 2.1×50 mm, 1.8 μm; flow rate 0.61 mL/min; temperature: 35° C. A: NH₄HCO₃ 10 mM, B: ACN; gradient: 0.3 min 98% A, 98% A to 100% B in 2.65 min; isocratic 2.05 min 100% B.

### Method C

Column ZORBAX Extend-C18 RRHD 2.1×50 mm, 1.8 μm; flow rate 0.61 mL/min; temperature: 35° C.; A: NH₄HCO₃ 10 mM, B: ACN, C: MeOH+0.1% formic acid; gradient: 0.3 min 98% A, 98% A to 0:95:5 A:B:C in 2.7 min; 0:95:5 A:B:C to 100% B in 0.1 min; isocratic 2 min 100% B.

### SYNTHESIS OF INTERMEDIATES

### Intermediate 1: (1 r,4r)-M-Benzyl-M-methylcyclohexane-1,4-diamine dihydrochloride

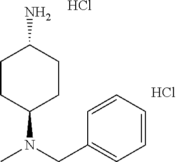 [SMILES: Cl ; CN(Cc1ccccc1)[C@H]1CC[C@H](N)CC1 ; Cl]

### Step 1. tert-Butyl ((1 r,4r)-4-(benzylamino)cyclohexyl)carbamate

To a solution of tert-butyl ((1r,4r)-4-aminocyclohexyl)carbamate (0.5 g, 2.33 mmol) in MeOH (15 mL), benzaldehyde (1.2 mL, 11.67 mmol) and AcOH (0.13 mL, 2.33 mmol) were added and the solution was stirred at rt overnight. Then, a mixture of NaBH₄ (0.88 g, 23.3 mmol) in MeOH (10 mL) was added and the reaction mixture was stirred at rt for 1 h. Then, it was cooled to 0° C. and 10 wt % NaOH aq. sol. (10 mL) was added. The organic solvent was evaporated and the remaining aqueous phase was extracted with DCM. The combined organic extracts were washed with brine, dried over MgSO₄, filtered and concentrated to dryness. The residue was purified by flash chromatography, silica gel, gradient DCM to MeOH:DCM (1:4), to give the title compound (0.48 g, 69% yield).

### Step 2. tert-Butyl ((1 r,4r)-4-(Benzyl(methyl)amino)cyclohexyl)carbamate

To a solution of the product obtained in Step 1 (0.48 g, 1.60 mmol) in MeOH (5 mL), formaldehyde (1.48 mL, 16.0 mmol) and AcOH (0.23 mL, 4.01 mmol) were added and the mixture was stirred at rt for 30 min. NaBH(OAc)₃ (0.85 g. 4.01 mmol) was added and reaction mixture was stirred at rt overnight. It was quenched with aq. NaHCO₃ sat. sol, and the aqueous phase was extracted with DCM. The combined organic extracts were washed with brine, dried over MgSO₄, filtered and concentrated to dryness to give the title compound (0.49 g, 98% yield).

### Step 3. Title Compound

To a solution of the product obtained in Step 2 (0.49 g, 1.56 mmol) in MeOH (36 mL), HCl solution (4 M in 1,4-dioxane, 1.95 mL, 7.82 mmol) was added and the reaction mixture was stirred at rt overnight. HPLC-MS analysis showed the presence of some residual starting material. Thus, additional HCl solution (4 M in 1,4-dioxane, 1.95 mL, 7.82 mmol) was added and the reaction mixture was again stirred at rt overnight. The solvent was concentrated to dryness to afford the title compound (0.44 g, 97% yield).

This method was used for the preparation of Intermediates 2 and 3 using suitable starting materials:

| Intermediate | STRUCTURE | CHEMICAL NAME |
|---|---|---|
| 2 | 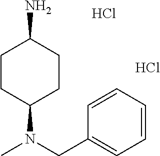 [SMILES: CN(Cc1ccccc1)[C@H]1CC[C@@H](N)CC1 ; Cl ; Cl] | (1s,4s)-N¹-Benzyl-N¹- methylcyclohexane- 1,4-diamine dihydrochloride |
| 3 | 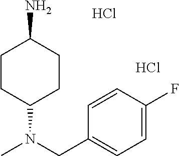 | (1r,4r)-N¹-(4- Fluorobenzyl)-N¹- methylcyclohexane- 1,4-diamine |

### Intermediate 4. 3-(3,3-Dimethylbutyl)-3,9-diazaspiro[5.5]undecane

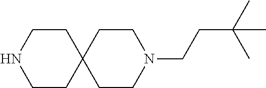 [SMILES: CC(C)(C)CCN1CCC2(CCNCC2)CC1]

### Step 1. tert-Butyl 9-(3,3-dimethylbutyl)-3,9-diazaspiro[5.5]undecane-3-carboxylate

To a solution of tert-butyl 3,9-diazaspiro[5.5]undecane-3-carboxylate (11.7 g, 46.0 mmol) in MeOH/AcOH (50 mL, 99:1 v %), 3,3-dimethylbutanal (4.92 mL, 48.2 mmol) was added and the resulting mixture was stirred for 1 h at rt. NaBH₃CN (5.77 g, 91.8 mmol) was added and the resulting mixture was stirred at rt for 18 h. All volatiles were removed under reduced pressure and the residue was partitioned between DCM and sat. aq. NaHCO₃ sol. The combined organic fractions were dried over Na₂SO₄ and the solvent removed under reduced pressure. The crude product was purified by Combiflash chromatography (120 g-SiO₂, column DCM/MeOH up to 40%) to give the title compound (12.5 g, 80% yield).

### Step 2a. 3-(3,3-Dimethylbutyl)-3,9-diazaspiro[5.5]undecane trifluoroacetate

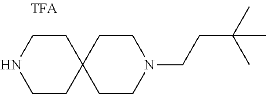 [SMILES: CC(C)(C)CCN1CCC2(CCNCC2)CC1]

The compound obtained in step 1 (4.73 g, 13.9 mmol) was dissolved in DCM (40 mL) and the reaction solution cooled to 0° C. At this temperature TFA was added (6.42 mL, 83.8 mmol) and the mixture was slowly allowed to reach rt and stirred overnight. The volatiles were removed under reduced pressure to give the title compound (6.7 g, quant. overweight, TFA salt) as viscous oil.

### Step 2b. 3-(3,3-Dimethylbutyl)-3,9-diazaspiro[5.5]undecane di hydrochloride

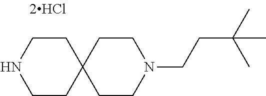 [SMILES: CC(C)(C)CCN1CCC2(CCNCC2)CC1]

A 2 M solution of HCl in EtOH (25 mL, 63 mmol) was added to a solution of the compound obtained in step 1 (4.28 g, 13 mmol) in MeOH (30 mL) and the mixture was stirred overnight. The volatiles were removed under reduced pressure to give the title compound (3.26 g, 83%).

This method was used for the preparation of Intermediates 5-13, that were obtained either as trifluoroacetate or dihydrochloride salt, using suitable starting materials:

| Inter- | | |
|---|---|---|
| mediate | STRUCTURE | CHEMICAL NAME |
| 5 | 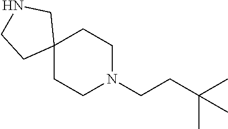 [SMILES: CC(C)(C)CCN1CCC2(CCNC2)CC1] | 8-(3,3- Dimethylbutyl)-2,8- diazaspiro[4.5]decane |
| 6 | 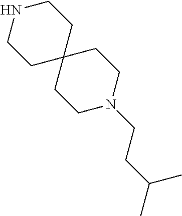 [SMILES: CC(C)CCN1CCC2(CCNCC2)CC1] | 3-Isopentyl-3,9- diazaspiro[5.5]undecane |
| 7 | 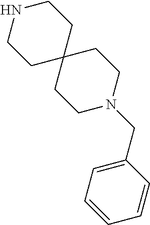 [SMILES: c1ccc(CN2CCC3(CCNCC3)CC2)cc1] | 3-Benzyl-3,9- diazaspiro[5.5]undecane |
| 8 | 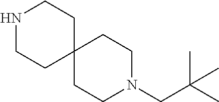 [SMILES: CC(C)(C)CN1CCC2(CCNCC2)CC1] | 3-Neopentyl-3,9- diazaspiro[5.5]undecane |
| 9 | 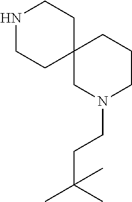 [SMILES: CC(C)(C)CCN1CCCC2(CCNCC2)C1] | 2-(3,3- Dimethylbutyl)-2,9- diazaspiro[5.5]undecane |
| 10 | 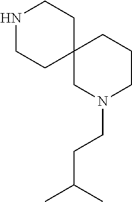 [SMILES: CC(C)CCN1CCCC2(CCNCC2)C1] | 2-Isopentyl-2,9- diazaspiro[5.5]undecane |
| 11 | 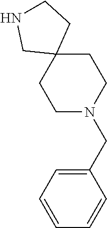 [SMILES: c1ccc(CN2CCC3(CCNC3)CC2)cc1] | 8-Benzyl-2,8- diazaspiro[4.5]decane |
| 12 | 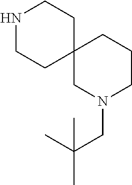 [SMILES: CC(C)(C)CN1CCCC2(CCNCC2)C1] | 2-Neopentyl-2,9- diazaspiro[5.5]undecane |
| 13 | 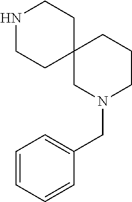 [SMILES: c1ccc(CN2CCCC3(CCNCC3)C2)cc1] | 2-Benzyl-2,9- diazaspiro[5.5]undecane |

### Intermediate 14. (R)-1-(6-Methoxypyridin-3-yl)-N-(piperidin-3-ylmethyl)methanamine trifluoroacetate

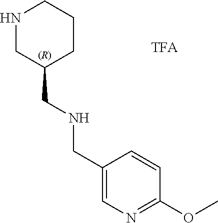

### Step 1. (S)-tert-Butyl 3-((((6-methoxypyridin-3-yl)methyl)amino)methyl)piperidine-1-carboxylate

To a solution of (S)-tert-butyl 3-(aminomethyl)piperidine-1-carboxylate (262 mg, 1.22 mmol) in DCM (5 mL), 6-methoxynicotinaldehyde (168 mg, 1.23 mmol) was added followed by a few drops of AcOH. NaBH(OAc)₃ (289 mg, 1.84 mmol) was added and the reaction stirred at rt overnight. The reaction was quenched with sat. aq. NaHCO₃ sol. and the product extracted with DCM. The combined organic fractions were dried over Na₂SO₄ and the solvent removed under reduced pressure. The crude product was purified by Combiflash chromatography (SiO₂, DCM/MeOH up to 10%) to give the title compound (200 mg, 49% yield).

### Step 2. Title Compound

The compound obtained in step 1 (200 mg, 0.6 mmol) was dissolved in DCM (8 mL) and the reaction solution cooled to 0° C. At this temperature TFA was added (456 μL, 5.96 mmol) and the mixture was slowly allowed to reach rt and stirred overnight. The volatiles were removed under reduced pressure to give the title compound (208 mg, quant., TFA salt).

This method was used for the preparation of Intermediate 15 using suitable starting materials.

| Intermediate | STRUCTURE | CHEMICAL NAME |
|---|---|---|
| 15 | 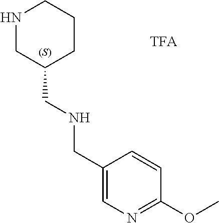 | (S)-1-(6- Methoxypyridin- 3-yl)-N- (piperidin-3- ylmethyl) methanamine 2,2,2- trifluoroacetate |

### Intermediate 16. 4-((2,9-Diazaspiro[5.5]undecan-2-yl)methyl)-2-fluorobenzonitrile dihydrochloride

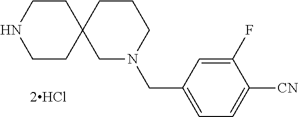 [SMILES: N#Cc1ccc(CN2CCCC3(CCNCC3)C2)cc1F]

### Step 1. tert-Butyl 2-(4-cyano-3-fluorobenzyl)-2,9-diazaspiro[5.5]undecane-9-carboxylate

tert-Butyl 2,9-diazaspiro[5.5]undecane-9-carboxylate (213 mg, 0.83 mmol), 4-(bromomethyl)-2-fluorobenzonitrile (200 mg, 0.92 mmol) and TEA (0.23 mL, 1.7 mmol) were placed in a sealed tube. ACN (10 mL) was added and the reaction mixture was stirred at r.t. for 16 h. Water and EtOAc were added, the layers were separated and the aqueous phase was extracted with EtOAc. The combined organic extracts were washed with brine, dried over MgSO₄, filtered and concentrated to dryness. The residue was purified by flash chromatography, silica gel, DCM:MeOH, to obtain the title compound (322 mg, 99% yield).

### Step 2. Title Compound

Starting from the compound obtained in step 1 (322 mg, 0.83 mmol) and following a similar procedure to that described in step 2b of intermediate 4, the title compound was obtained (300 mg, Quant.).

This method was used for the preparation of Intermediates 17-19 using suitable starting materials:

| Intermediate | STRUCTURE | CHEMICAL NAME |
|---|---|---|
| 17 | 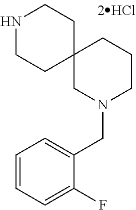 [SMILES: Fc1ccccc1CN1CCCC2(CCNCC2)C1] | 2-(2-Fluorobenzyl)-2,9- diazaspiro[5.5]undecane dihydrochloride |
| 18 | 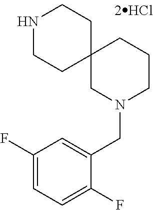 [SMILES: Fc1ccc(F)c(CN2CCCC3(CCNCC3)C2)c1] | 2-(2,5-Difluorobenzyl)-2,9- diazaspiro[5.5]undecane dihydrochloride |
| 19 | 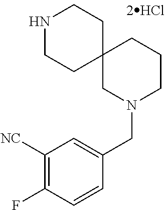 [SMILES: N#Cc1cc(CN2CCCC3(CCNCC3)C2)ccc1F] | 5-((2,9-Diazaspiro [5.5]undecan-2- yl)methyl)-2- fluorobenzonitrile dihydrochloride |

### Intermediate 20. 2-((Tetrahydro-2H-pyran-4-yl)methyl)-2,9-diazaspiro[5.5]undecanedihydrochloride

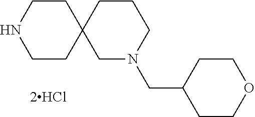 [SMILES: C1CN(CC2CCOCC2)CC2(C1)CCNCC2]

### Step 1. tert-Butyl 2-((tetrahydro-2H-pyran-4-yl)methyl)-2,9-diazaspiro[5.5]undecane-9-carboxylate

To a solution of tert-butyl 2,9-diazaspiro[5.5]undecane-9-carboxylate (250 mg, 1 mmol) in DOE (5 mL), tetrahydro-2H-pyran-4-carbaldehydes (174 mg, 1.53 mmol) and AcOH (0.06 mL, 1 mmol) were added, and the mixture was stirred at rt for 5 min. NaBH(OAc)₃ (417 g. 2 mmol) was added and the reaction mixture was stirred at 50° C. for 90 min. It was quenched with aq. NaHCO₃ sat. sol. and the aqueous phase was extracted with DCM. The combined organic extracts were washed with brine, dried over MgSO₄, filtered and concentrated to dryness to give the title compound (351 mg, Quant.).

### Step 2. Title Compound

A 2 M solution of HCl in EtOH (11.8 mL, 15 mmol) was added to a solution of the compound obtained in step 1 (351 mg, 1 mmol) in MeOH (20 mL) and the mixture was stirred overnight. The volatiles were removed under reduced pressure and the residue was suspended in Et₂O. The resulting solid was filtered to give the title compound (331 mg, 98% yield).

This method was used for the preparation of Intermediate 21 using suitable starting materials:

| Intermediate | STRUCTURE | CHEMICAL NAME |
|---|---|---|
| 21 | 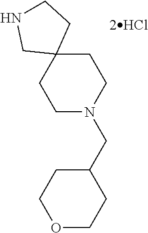 [SMILES: C1CC2(CCN(CC3CCOCC3)CC2)CN1] | 8-((Tetrahydro-2H-pyran-4- yl)methyl)-2,8- diazaspiro[4.5]decane dihydrochloride |

### SYNTHESIS OF EXAMPLES

### Example 1. (S)—N-(1-Benzylpyrrolidin-3-yl)-6-morpholinopyridine-3-sulfonamide

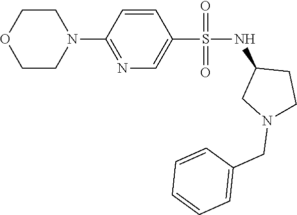 [SMILES: O=S(=O)(N[C@H]1CCN(Cc2ccccc2)C1)c1ccc(N2CCOCC2)nc1]

A mixture of 6-morpholinopyridine-3-sulfonyl chloride (100 mg, 0.38 mmol), and (S)-1-benzylpyrrolidin-3-amine (67 mg, 0.38 mmol) in pyridine (1.5 mL) was placed in a microwave vial. The system was purged with vacuum/N₂ cycles and it was irradiated under microwave heating at 130° C. for 5 min. After cooling, aq. NaHCO₃ sat. sol. and DCM were added to the reaction mixture. The phases were separated and the aqueous layer was extracted with DCM. The combined organic extracts were washed with brine, dried over MgSO₄, filtered and concentrated to dryness. The residue was purified by flash chromatography, silica gel, gradient DCM to MeOH:DCM (1:4), to give the title compound (78 mg, 51% yield).

HPLC Rt (Method A): 4.24 min; ESI+-MS m/z: 403.2 (M+H)⁺.

This method was used for the preparation of Examples 2-12 using suitable starting materials:

| | | | Rt | MS | HPLC |
|---|---|---|---|---|---|
| EX | STRUCTURE | CHEMICAL NAME | (min) | (M + H) | Method |
| 2 | 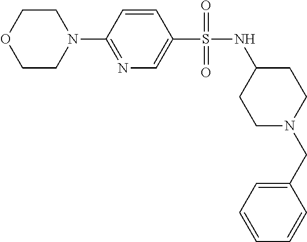 [SMILES: O=S(=O)(NC1CCN(Cc2ccccc2)CC1)c1ccc(N2CCOCC2)nc1] | N-(1-Benzylpiperidin-4-yl)-6- morpholinopyridine-3-sulfonamide | 4.39 | 417.2 | A |
| 3 | 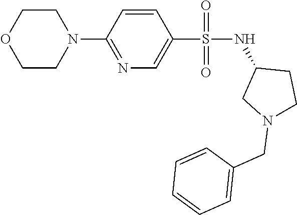 [SMILES: O=S(=O)(N[C@@H]1CCN(Cc2ccccc2)C1)c1ccc(N2CCOCC2)nc1] | (R)-N-(1-Benzylpyrrolidin-3-yl)-6- morpholinopyridine-3-sulfonamide | 4.23 | 403.2 | A |
| 4 | 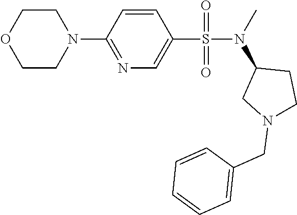 [SMILES: CN([C@H]1CCN(Cc2ccccc2)C1)S(=O)(=O)c1ccc(N2CCOCC2)nc1] | (S)-N-(1-Benzylpyrrolidin-3-yl)-N- methyl-6-morpholinopyridine-3- sulfonamide | 4.87 | 417.2 | A |
| 5 | 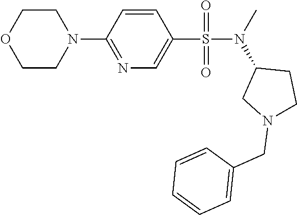 [SMILES: CN([C@@H]1CCN(Cc2ccccc2)C1)S(=O)(=O)c1ccc(N2CCOCC2)nc1] | (R)-N-(1-Benzylpyrrolidin-3-yl)-N- methyl-6-morpholinopyridine-3- sulfonamide | 4.87 | 417.1 | A |
| 6 | 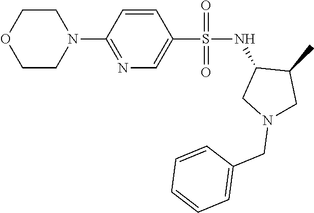 [SMILES: C[C@H]1CN(Cc2ccccc2)C[C@@H]1NS(=O)(=O)c1ccc(N2CCOCC2)nc1] | N-((3R,4S)-1-Benzyl-4- methylpyrrolidin-3-yl)-6- morpholinopyridine-3-sulfonamide | 4.54 | 417.2 | A |
| 7 | 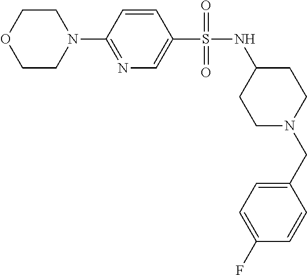 [SMILES: O=S(=O)(NC1CCN(Cc2ccc(F)cc2)CC1)c1ccc(N2CCOCC2)nc1] | N-(1-(4-Fluorobenzyl)piperidin-4- yl)-6-morpholinopyridine-3- sulfonamide | 4.41 | 435.1 | A |
| 8 | 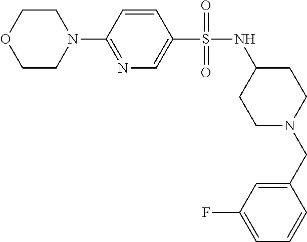 [SMILES: O=S(=O)(NC1CCN(Cc2cccc(F)c2)CC1)c1ccc(N2CCOCC2)nc1] | N-(1-(3-Fluorobenzyl)piperidin-4- yl)-6-morpholinopyridine-3- sulfonamide | 4.86 | 435.1 | A |
| 9 | 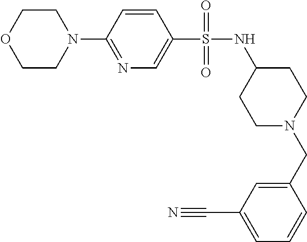 [SMILES: N#Cc1cccc(CN2CCC(NS(=O)(=O)c3ccc(N4CCOCC4)nc3)CC2)c1] | N-(1-(3-Cyanobenzyl)piperidin-4- yl)-6-morpholinopyridine-3- sulfonamide | 4.22 | 442.1 | A |
| 10 | 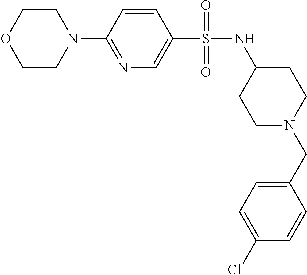 [SMILES: O=S(=O)(NC1CCN(Cc2ccc(Cl)cc2)CC1)c1ccc(N2CCOCC2)nc1] | N-(1-(4-Chlorobenzyl)piperidin-4- yl)-6-morpholinopyridine-3- sulfonamide | 4.77 | 451.0 | A |
| 11 | 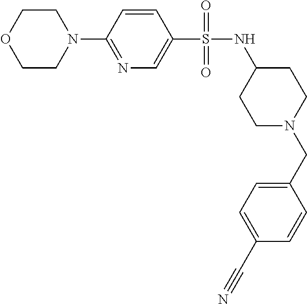 [SMILES: N#Cc1ccc(CN2CCC(NS(=O)(=O)c3ccc(N4CCOCC4)nc3)CC2)cc1] | N-(1-(4-Cyanobenzyl)piperidin-4- yl)-6-morpholinopyridine-3- sulfonamide | 4.23 | 442.1 | A |
| 12 | 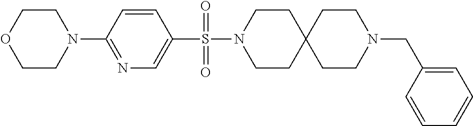 [SMILES: O=S(=O)(c1ccc(N2CCOCC2)nc1)N1CCC2(CCN(Cc3ccccc3)CC2)CC1] | 4-(5-((9-Benzyl-3,9- diazaspiro[5.5]undecan-3- yl)sulfonyl)pyridin-2-yl)morpholine | 5.04 | 471.1 | A |

### Example 13. 4-(5-((2-Benzyl-2,8-diazaspiro[4.5]decan-8-yl)sulfonyl)pyridin-2-yl)morpholine

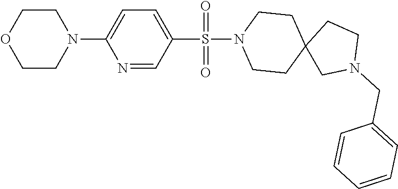 [SMILES: O=S(=O)(c1ccc(N2CCOCC2)nc1)N1CCC2(CCN(Cc3ccccc3)C2)CC1]

A solution of 6-morpholinopyridine-3-sulfonyl chloride (50 mg, 0.19 mmol), 2-benzyl-2,8-diazaspiro[4.5]decane dihydrochloride (58 mg, 0.19 mmol) and TEA (0.05 mL, 0.38 mmol) in DCM (1.5 mL) was stirred at rt overnight. The reaction mixture was diluted with DCM and it was washed with 1 N aq. NaOH sol. The aqueous phase was extracted with DCM. The combined organic extracts were washed with brine, dried over MgSO₄, filtered and concentrated to dryness. The residue was purified by flash chromatography, C₁₈, gradient NH₄HCO₃ pH 8 to ACN, to obtain the title compound (45 mg, 51% yield).

HPLC Rt (Method A): 5.03 min; ESI+-MS m/z: 457.1 (M+H)⁺.

This method was used for the preparation of Examples 14-19 using suitable starting materials and intermediates 1-6:

| | | | Rt | MS | HPLC |
|---|---|---|---|---|---|
| EX | STRUCTURE | CHEMICAL NAME | (min) | (M + H) | Method |
| 14 | 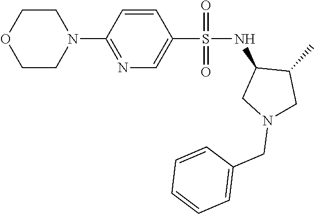 [SMILES: C[C@@H]1CN(Cc2ccccc2)C[C@H]1NS(=O)(=O)c1ccc(N2CCOCC2)nc1] | N-((3S,4R)-1-Benzyl-4- methylpyrrolidin-3-yl)-6- morpholinopyridine-3-sulfonamide | 4.55 | 417.1 | A |
| 15 | 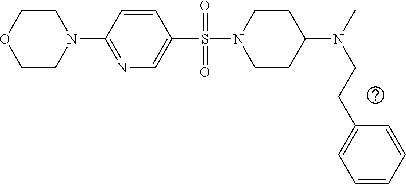 [SMILES: CN(CCc1ccccc1)C1CCN(S(=O)(=O)c2ccc(N3CCOCC3)nc2)CC1] | N-Methyl-1-((6-morpholinopyridin- 3-yl)sulfonyl)-N- phenethylpiperidin-4-amine | 4.86 | 445.1 | A |
| 16 | 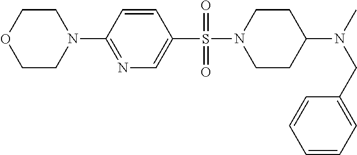 [SMILES: CN(Cc1ccccc1)C1CCN(S(=O)(=O)c2ccc(N3CCOCC3)nc2)CC1] | N-Benzyl-N-methyl-1-((6- morpholinopyridin-3- yl)sulfonyl)piperidin-4-amine | 4.93 | 431.1 | A |
| 17 | 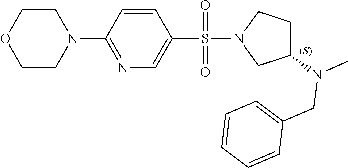 | (S)-N-Benzyl-N-methyl-1-((6- morpholinopyridin-3- yl)sulfonyl)pyrrolidin-3-amine | 4.75 | 417.1 | A |
| 18 | 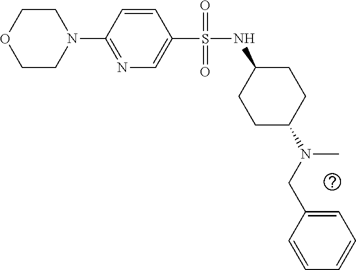 [SMILES: CN(Cc1ccccc1)[C@H]1CC[C@H](NS(=O)(=O)c2ccc(N3CCOCC3)nc2)CC1] | N-((1r,4r)-4- (Benzyl(methyl)amino)cyclohexyl)- 6-morpholinopyridine-3- sulfonamide | 4.49 | 445.1 | A |
| 19 | 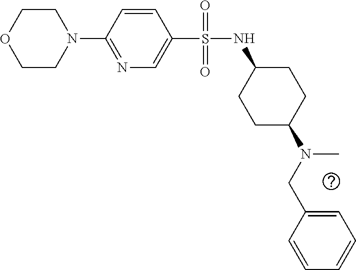 [SMILES: CN(Cc1ccccc1)[C@H]1CC[C@@H](NS(=O)(=O)c2ccc(N3CCOCC3)nc2)CC1] | N-((1s,4s)-4- (Benzyl(methyl)amino)cyclohexyl)- 6-morpholinopyridine-3- sulfonamide | 4.64 | 445.2 | A |

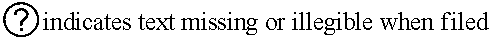

### Example 20. N—((S)-1-Benzylpyrrolidin-3-yl)-6-((2R,6S)-2,6-dimethylmorpholino)pyridine-3-sulfonamide

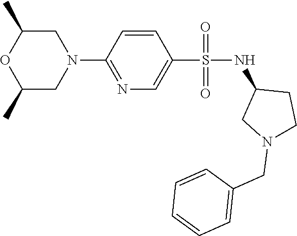 [SMILES: C[C@@H]1CN(c2ccc(S(=O)(=O)N[C@H]3CCN(Cc4ccccc4)C3)cn2)C[C@H](C)O1]

### Step 1. (S)—N-(1-Benzylpyrrolidin-3-yl)-6-chloropyridine-3-sulfonamide

A solution of 6-chloropyridine-3-sulfonyl chloride (1.0 g, 4.72 mmol), (S)-1-benzylpyrrolidin-3-amine (831 mg, 4.72 mmol) and TEA (0.8 mL, 5.66 mmol) in DCM (5 mL) was stirred at rt overnight. Additional 6-chloropyridine-3-sulfonyl chloride (0.5 g, 2.36 mmol) was added and the reaction mixture was again stirred at rt overnight. The reaction mixture was poured into water and it was extracted with DCM. The combined organic extracts were washed with brine, dried over MgSO₄, filtered and concentrated to dryness. The residue was purified by flash chromatography, silica gel, gradient DCM to MeOH:DCM (1:4), to give the title compound (0.806 g, 48% yield).

### Step 2. Title Compound

A mixture of the product obtained in Step 1 (100 mg, 0.28 mmol), cis-2,6-dimethylmorpholine (164 mg, 1.42 mmol) and TEA (0.2 mL, 1.42 mmol) in EtOH (5 mL) was placed in a microwave vial. The system was purged with vacuum/N₂ cycles and it was irradiated under microwave heating at 120° C. for 1 h. After cooling down to rt, the solvent was evaporated to dryness. The residue was dissolved in DCM and it was washed with 1 N aq. NaOH sol. The aqueous phase was back extracted with DCM. The combined organic extracts were washed with brine, dried over MgSO₄, filtered and evaporated to dryness. The residue was purified by flash chromatography, silica gel, gradient DCM to MeOH:DCM (1:4), to give the title compound (68 mg, 56% yield).

HPLC Rt (Method A): 4.81 min; ESI+-MS m/z: 431.2 (M+H)⁺.

This method was used for the preparation of Examples 21-34 using suitable starting materials and intermediate 1-6

| | | | Rt | MS | HPLC |
|---|---|---|---|---|---|
| EX | STRUCTURE | CHEMICAL NAME | (min) | (M + H) | Method |
| 21 | 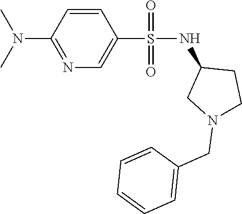 [SMILES: CN(C)c1ccc(S(=O)(=O)N[C@H]2CCN(Cc3ccccc3)C2)cn1] | (S)-N-(1- Benzylpyrrolidin- 3-yl)-6- (dimethylamino) pyridine-3- sulfonamide⁽¹⁾ | 4.33 | 361.1 | A |
| 22 | 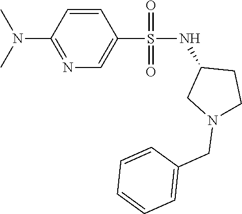 [SMILES: CN(C)c1ccc(S(=O)(=O)N[C@@H]2CCN(Cc3ccccc3)C2)cn1] | (R)-N-(1- Benzylpyrrolidin- 3-yl)-6- (dimethylamino) pyridine-3- sulfonamide⁽¹⁾ | 4.32 | 361.1 | A |
| 23 | 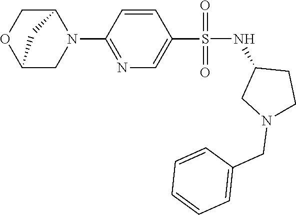 [SMILES: O=S(=O)(N[C@@H]1CCN(Cc2ccccc2)C1)c1ccc(N2C[C@@H]3C[C@H]2CO3)nc1] | N-((R)-1- Benzylpyrrolidin- 3-yl)-6-((1S,4S)- 2-oxa-5- azabicyclo[2.2.1] heptan-5- yl)pyridine-3- sulfonamide | 3.99 | 415.1 | A |
| 24 | 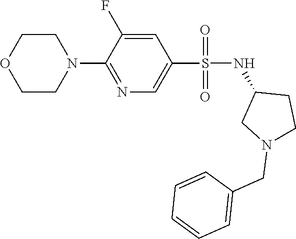 [SMILES: O=S(=O)(N[C@@H]1CCN(Cc2ccccc2)C1)c1cnc(N2CCOCC2)c(F)c1] | (R)-N-(1- Benzylpyrrolidin-3- yl)-5-fluoro-6- morpholinopyridine-3- sulfonamide | 4.61 | 421.1 | A |
| 25 | 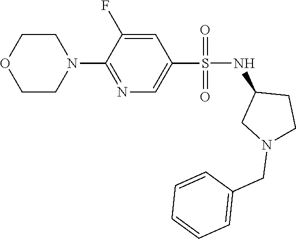 [SMILES: O=S(=O)(N[C@H]1CCN(Cc2ccccc2)C1)c1cnc(N2CCOCC2)c(F)c1] | (S)-N-(1- Benzylpyrrolidin-3- yl)-5-fluoro-6- morpholinopyridine-3- sulfonamide | 4.60 | 421.1 | A |
| 26 | 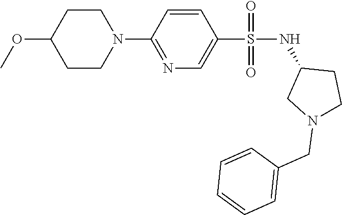 [SMILES: COC1CCN(c2ccc(S(=O)(=O)N[C@@H]3CCN(Cc4ccccc4)C3)cn2)CC1] | (R)-N-(1- Benzylpyrrolidin- 3-yl)-6-(4- methoxypiperidin- 1-yl)pyridine- 3-sulfonamide | 4.61 | 431.1 | A |
| 27 | 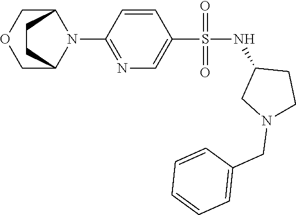 [SMILES: O=S(=O)(N[C@@H]1CCN(Cc2ccccc2)C1)c1ccc(N2[C@@H]3CC[C@H]2COC3)nc1] | N-((R)-1- Benzylpyrrolidin- 3-yl)-6-((1R,5S)- 3-oxa-8-azabicyclo [3.2.1]octan-8- yl)pyridine-3- sulfonamide | 4.44 | 429.1 | A |
| 28 | 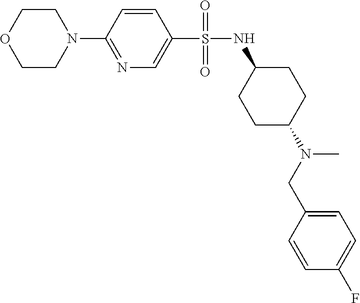 [SMILES: CN(Cc1ccc(F)cc1)[C@H]1CC[C@H](NS(=O)(=O)c2ccc(N3CCOCC3)nc2)CC1] | N-((1r,4r)-4-((4- Fluorobenzyl)(methyl) amino)cyclohexyl)-6- morpholinopyridine- 3-sulfonamide | 1.96 | 463.2 | B |
| 29 | 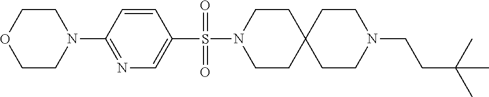 [SMILES: CC(C)(C)CCN1CCC2(CC1)CCN(S(=O)(=O)c1ccc(N3CCOCC3)nc1)CC2] | 4-(5-((9-(3,3- Dimethylbutyl)-3,9- diazaspiro[5.5] undecan-3- yl)sulfonyl)pyridin- 2-yl)morpholine | 2.00 | 465.2 | B |
| 30 | 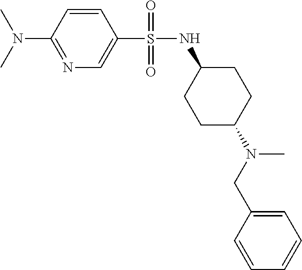 [SMILES: CN(C)c1ccc(S(=O)(=O)N[C@H]2CC[C@H](N(C)Cc3ccccc3)CC2)cn1] | N-((1r,4r)-4-(Benzyl (methyl)amino) cyclohexyl)- 6-(dimethylamino) pyridine-3- sulfonamide | 1.97 | 403.2 | B |
| 31 | 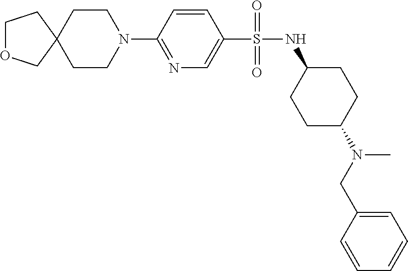 [SMILES: CN(Cc1ccccc1)[C@H]1CC[C@H](NS(=O)(=O)c2ccc(N3CCC4(CCOC4)CC3)nc2)CC1] | N-((1r,4r)-4- (Benzyl(methyl) amino)cyclohexyl)- 6-(1-oxa- 8-azaspiro[4.5] decan-8- yl)pyridine-3- sulfonamide | 2.09 | 499.2 | B |
| 32 | 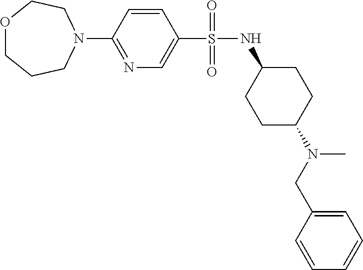 [SMILES: CN(Cc1ccccc1)[C@H]1CC[C@H](NS(=O)(=O)c2ccc(N3CCCOCC3)nc2)CC1] | N-((1r,4r)-4- (Benzyl(methyl) amino) cyclohexyl)-6-(1,4- oxazepan-4-yl) pyridine-3- sulfonamide | 1.94 | 459.2 | B |
| 33 | 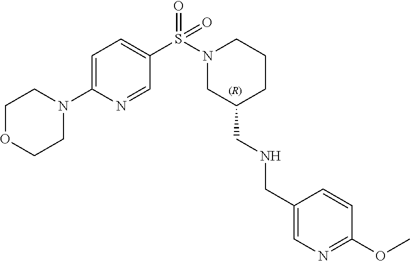 | (R)-1-(6- Methoxypyridin-3- yl)-N-((1-((6- morpholinopyridin-3- yl)sulfonyl) piperidin-3- yl)methyl) methanamine | 1.78 | 462.2 | B |
| 34 | 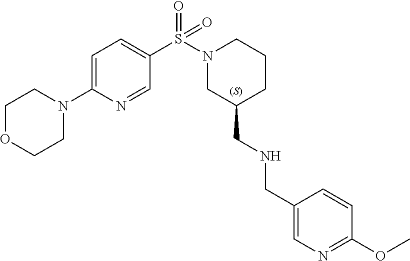 | (S)-1-(6- Methoxypyridin-3- yl)-N-((1-((6- morpholinopyridin-3- yl)sulfonyl) piperidin-3- yl)methyl) methanamine | 1.76 | 462.2 | B |

⁽¹⁾Step 2 was performed using dimethylamine solution (2M in THF) and THF as solvent heating at 90° C. under conventional thermal heating

### Example 35. (S)—N-(1-(3-Fluorobenzyl)pyrrolidin-3-yl)-6-morpholinopyridine-3-sulfonamide

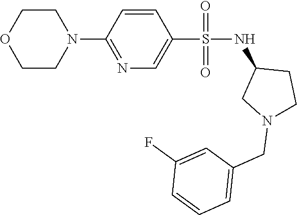 [SMILES: O=S(=O)(N[C@H]1CCN(Cc2cccc(F)c2)C1)c1ccc(N2CCOCC2)nc1]

### Step 1. (S)-tert-Butyl 3-(6-morpholinopyridine-3-sulfonamido)pyrrolidine-1-carboxylate

Following the experimental procedure described in Example 1, starting from tert-butyl (S)-3-aminopyrrolidine-1-carboxylate (0.57 g, 3.05 mmol) and 6-morpholinopyridine-3-sulfonyl chloride (0.80 g, 3.05 mmol), the title compound was obtained (0.64 g, 51% yield).

### Step 2. (S)-6-Morpholino-N-(pyrrolidin-3-yl)pyridine-3-sulfonamide trifluoroacetate

A solution of the product obtained in Step 1 (0.64 g, 1.55 mmol) and TFA (1.2 mL, 15.51 mmol) in DCM (2 mL) was stirred at rt overnight. The solvent was evaporated to dryness to afford the title compound (1.87 g, overweight, 35 wt %; quant. yield was assumed) as a crude product that was used as such without further purification Step 3. Title compound.

To a solution of the crude product obtained in Step 2 (165 mg, 35 wt %, 0.14 mmol), 3-fluorobenzaldehyde (0.015 mL, 0.14 mmol) and DIPEA (0.025 mL, 0.14 mmol) in DCM (7 mL), cooled at 0° C., NaBH(OAc)₃ (29 mg, 0.14 mmol) was added. The reaction mixture was stirred at rt overnight. Water and aq. NaHCO₃ sat. sol. were added and it was extracted with DCM. The combined organic extracts were washed with brine, dried over MgSO₄, filtered and concentrated to dryness. The residue was purified by flash chromatography, silica gel, gradient DCM to MeOH:DCM (1:4), to give the title compound (39 mg, 68% yield).

HPLC Rt (Method A): 4.42 min; ESI+-MS m/z: 421.1 (M+H)⁺.

This method was used for the preparation of Examples 36-48 using suitable starting materials:

| | | | Rt | MS | HPLC |
|---|---|---|---|---|---|
| EX | STRUCTURE | CHEMICAL NAME | (min) | (M + H) | Method |
| 36 | 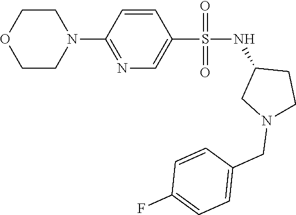 [SMILES: O=S(=O)(N[C@@H]1CCN(Cc2ccc(F)cc2)C1)c1ccc(N2CCOCC2)nc1] | (R)-N-(1-(4- Fluorobenzyl) pyrrolidin-3-yl)-6- morpholinopyridine- 3-sulfonamide | 4.36 | 421.1 | A |
| 37 | 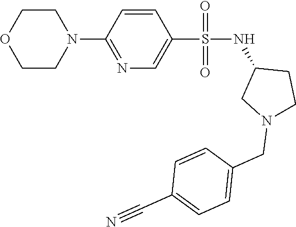 [SMILES: N#Cc1ccc(CN2CC[C@@H](NS(=O)(=O)c3ccc(N4CCOCC4)nc3)C2)cc1] | (R)-N-(1-(4- Cyanobenzyl) pyrrolidin-3-yl)-6- morpholinopyridine- 3-sulfonamide | 4.14 | 428.2 | A |
| 38 | 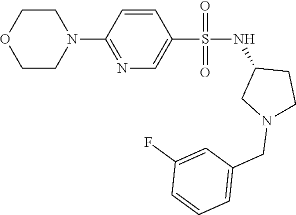 [SMILES: O=S(=O)(N[C@@H]1CCN(Cc2cccc(F)c2)C1)c1ccc(N2CCOCC2)nc1] | (R)-N-(1-(3- Fluorobenzyl) pyrrolidin-3-yl)-6- morpholinopyridine- 3-sulfonamide | 4.42 | 421.1 | A |
| 39 | 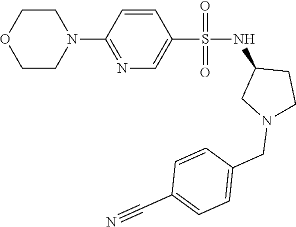 [SMILES: N#Cc1ccc(CN2CC[C@H](NS(=O)(=O)c3ccc(N4CCOCC4)nc3)C2)cc1] | (S)-N-(1-(4- Cyanobenzyl) pyrrolidin-3-yl)-6- morpholinopyridine- 3-sulfonamide | 4.14 | 428.2 | A |
| 40 | 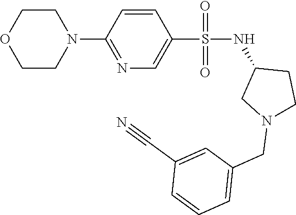 [SMILES: N#Cc1cccc(CN2CC[C@@H](NS(=O)(=O)c3ccc(N4CCOCC4)nc3)C2)c1] | (R)-N-(1-(3- Cyanobenzyl) pyrrolidin-3-yl)-6- morpholinopyridine- 3-sulfonamide | 4.14 | 428.2 | A |
| 41 | 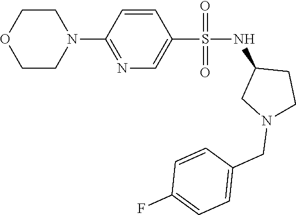 [SMILES: O=S(=O)(N[C@H]1CCN(Cc2ccc(F)cc2)C1)c1ccc(N2CCOCC2)nc1] | (S)-N-(1-(4- Fluorobenzyl) pyrrolidin-3-yl)-6- morpholinopyridine- 3-sulfonamide | 4.35 | 421.1 | A |
| 42 | 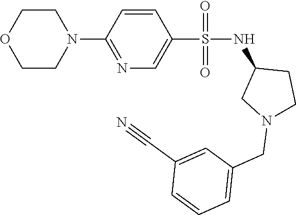 [SMILES: N#Cc1cccc(CN2CC[C@H](NS(=O)(=O)c3ccc(N4CCOCC4)nc3)C2)c1] | (S)-N-(1-(3- Cyanobenzyl) pyrrolidin-3-yl)-6- morpholinopyridine- 3-sulfonamide | 4.14 | 428.1 | A |
| 43 | 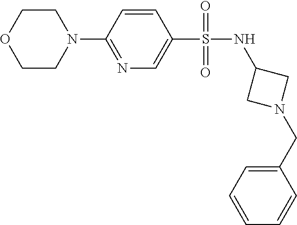 [SMILES: O=S(=O)(NC1CN(Cc2ccccc2)C1)c1ccc(N2CCOCC2)nc1] | N-(1-Benzylazetidin- 3-yl)-6- morpholinopyridine- 3-sulfonamide | 3.91 | 389.0 | A |
| 44 | 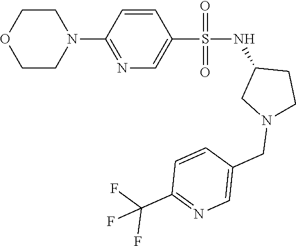 [SMILES: O=S(=O)(N[C@@H]1CCN(Cc2ccc(C(F)(F)F)nc2)C1)c1ccc(N2CCOCC2)nc1] | (R)-6-Morpholino- N-(1-((6- (trifluoromethyl) pyridin-3- yl)methyl)pyrrolidin- 3-yl)pyridine- 3-sulfonamide | 4.24 | 472.0 | A |
| 45 | 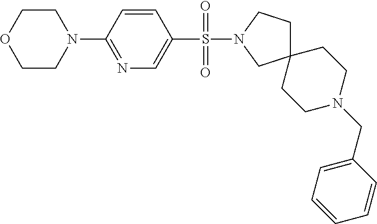 [SMILES: O=S(=O)(c1ccc(N2CCOCC2)nc1)N1CCC2(CCN(Cc3ccccc3)CC2)C1] | 4-(5-((8-Benzyl-2,8- diazaspiro[4.5]decan-2- yl)sulfonyl)pyridin-2- yl)morpholine⁽¹⁾ | 4.87 | 457.1 | A |
| 46 | 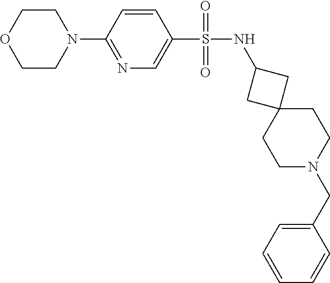 [SMILES: O=S(=O)(NC1CC2(CCN(Cc3ccccc3)CC2)C1)c1ccc(N2CCOCC2)nc1] | N-(7-Benzyl-7- azaspiro[3.5] nonan-2-yl)-6- morpholinopyridine-3- sulfonamide⁽¹⁾ | 4.55 | 457.1 | A |
| 47 | 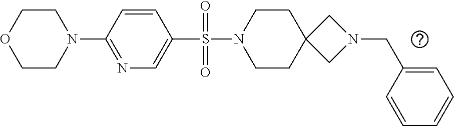 [SMILES: O=S(=O)(c1ccc(N2CCOCC2)nc1)N1CCC2(CC1)CN(Cc1ccccc1)C2] | 4-(5-((2-Benzyl-2,7- diazaspiro[3.5]nonan-7- yl)sulfonyl)pyridin- 2-yl)morpholine | 4.61 | 443.2 | A |
| 48 | 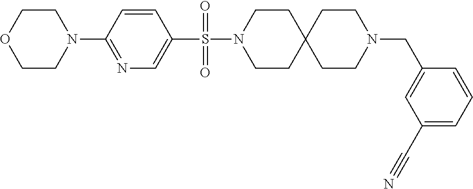 [SMILES: N#Cc1cccc(CN2CCC3(CC2)CCN(S(=O)(=O)c2ccc(N4CCOCC4)nc2)CC3)c1] | 3-((9-((6- Morpholinopyridin-3- yl)sulfonyl)-3,9- diazaspiro[5.5] undecan-3- yl)methyl)benzonitrile | 4.99 | 496.2 | A |

⁽¹⁾Step 1 was performed following the experimental procedure described in Example 15

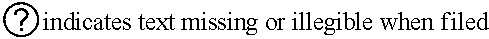

### Example 49. (R)—N-(1-(4-Cyanobenzyl)pyrrolidin-3-yl)-N-isopropyl-6-morpholinopyridine-3-sulfonamide

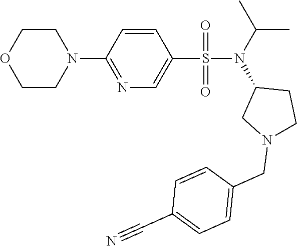 [SMILES: CC(C)N([C@@H]1CCN(Cc2ccc(C#N)cc2)C1)S(=O)(=O)c1ccc(N2CCOCC2)nc1]

### Step 1. (R)—N-Isopropyl-6-morpholino-N-(pyrrolidin-3-yl)pyridine-3-sulfonamide

A mixture of 6-morpholinopyridine-3-sulfonyl chloride (230 mg, 0.87 mmol) and tert-butyl (R)-3-(isopropylamino)pyrrolidine-1-carboxylate (200 mg, 0.87 mmol) in pyridine (0.5 mL) was placed in a microwave vial. The system was purged with vacuum/N₂ cycles and it was irradiated under microwave heating at 130° C. for 2 h. Additional 6-morpholinopyridine-3-sulfonyl chloride (115 mg, 0.43 mmol) was added and the system was again irradiated under microwave heating at 130° C. for 1 h. After cooling, aq. NaHCO₃ sat. sol. and DCM were added to the reaction mixture. The phases were separated and the aqueous layer was extracted with DCM. The combined organic extracts were washed with brine, dried over MgSO₄, filtered and concentrated to dryness. The residue was purified by flash chromatography, silica gel, gradient DCM to MeOH:DCM (1:4), to give the title compound (135 mg, 43% yield).

### Step 2. Title Compound

The product obtained in Step 1 (65 mg, 0.18 mmol), 4-(bromomethyl)benzonitrile (36 mg, 0.18 mmol) and K₂CO₃ (51 mg, 0.37 mmol) were placed in a sealed tube. ACN (2 mL) was added and the reaction mixture was stirred at 80° C. for 48 h. Water and EtOAc were added, the layers were separated and the aqueous phase was extracted with EtOAc. The combined organic extracts were washed with brine, dried over MgSO₄, filtered and concentrated to dryness. The residue was purified by flash chromatography, silica gel, gradient DCM to MeOH:DCM (1:4), and then it was further purified by flash chromatography, C₁₈, gradient NH₄HCO₃ pH 8 to ACN, to obtain the title compound (16 mg, 18% yield).

HPLC Rt (Method A): 5.34 min; ESI+-MS m/z: 470.0 (M+H)⁺.

This method was used for the preparation of Example 50 using suitable starting materials:

| | | | Rt | MS | HPLC |
|---|---|---|---|---|---|
| EX | STRUCTURE | CHEMICAL NAME | (min) | (M + H) | Method |
| 50 | 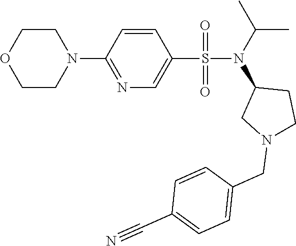 [SMILES: CC(C)N([C@H]1CCN(Cc2ccc(C#N)cc2)C1)S(=O)(=O)c1ccc(N2CCOCC2)nc1] | (S)-N-(1-(4- Cyanobenzyl)pyrrolidin-3-yl)-N- isopropyl-6-morpholinopyridine-3- sulfonamide | 5.29 | 470.0 | A |

### Example 51. N-((1 r,4r)-4-(Benzylamino)cyclohexyl)-6-morpholinopyridine-3-sulfonamide

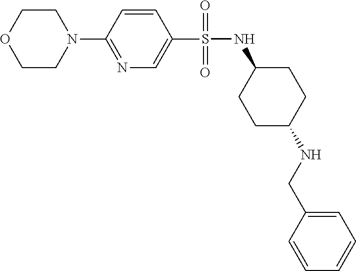 [SMILES: O=S(=O)(N[C@H]1CC[C@H](NCc2ccccc2)CC1)c1ccc(N2CCOCC2)nc1]

### Step 1. tert-Butyl ((1r,4r)-4-(6-morpholinopyridine-3-sulfonamido)cyclohexyl)carbamate

Following the procedure described in example 13 using suitable starting material the title compound (467 mg, 66% yield) was obtained.

### Step 2. N-((1 r,4r)-4-Aminocyclohexyl)-6-morpholinopyridine-3-sulfonamide trifluoroacetate

Starting from compound obtained in step 1 using the procedure described in example 35 step 2, the title compound was obtained quant. as TFA salt.

### Step 3. Title Compound

Starting from the base of compound obtained in step 2 (liberating with triethylamine) following a similar procedure to that described in intermediate 1, step 1, the title compound (24 mg, 49% yield) was obtained.

HPLC Rt (Method B): 1.61 min; ESI+-MS m/z: 431.2 (M+H)⁺.

This method was used for the preparation of Examples 52-55 using suitable starting materials

| | | | Rt | MS | HPLC |
|---|---|---|---|---|---|
| EX | STRUCTURE | CHEMICAL NAME | (min) | (M + H) | Method |
| 52 | 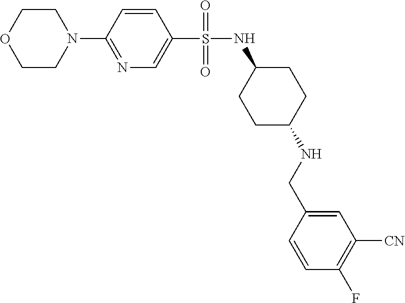 [SMILES: N#Cc1cc(CN[C@H]2CC[C@H](NS(=O)(=O)c3ccc(N4CCOCC4)nc3)CC2)ccc1F] | N-((1r,4r)-4-((3-Cyano-4- fluorobenzyl)amino) cyclohexyl)-6- morpholinopyridine- 3-sulfonamide | 1.71 | 474.0 | B |
| 53 | 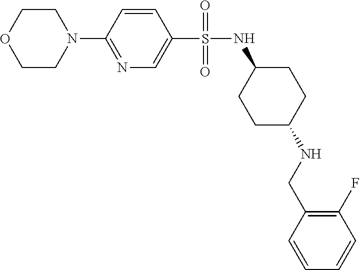 [SMILES: O=S(=O)(N[C@H]1CC[C@H](NCc2ccccc2F)CC1)c1ccc(N2CCOCC2)nc1] | N-((1r,4r)-4-((2- Fluorobenzyl)amino)cyclohexyl)- 6-morpholinopyridine-3- sulfonamide | 1.70 | 449.0 | B |
| 54 | 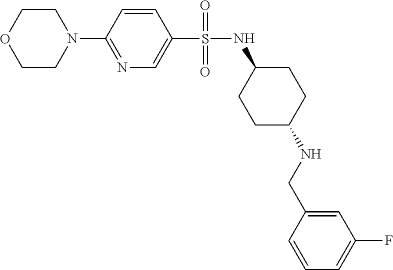 [SMILES: O=S(=O)(N[C@H]1CC[C@H](NCc2cccc(F)c2)CC1)c1ccc(N2CCOCC2)nc1] | N-((1r,4r)-4-((3- Fluorobenzyl)amino)cyclohexyl)- 6-morpholinopyridine-3- sulfonamide | 1.73 | 449.0 | B |
| 55 | 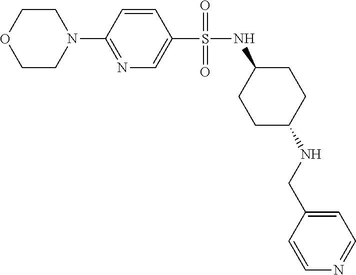 [SMILES: O=S(=O)(N[C@H]1CC[C@H](NCc2ccncc2)CC1)c1ccc(N2CCOCC2)nc1] | 6-Morpholino-N-((1r,4r)-4- ((pyridin-4-ylmethyl) amino)cyclohexyl)pyridine- 3-sulfonamide | 1.31 | 432.2 | B |

### Example 56. N-((1r,4r)-4-((2-Fluorobenzyl)(methyl)amino)cyclohexyl)-6-morpholinopyridine-3-sulfonamide

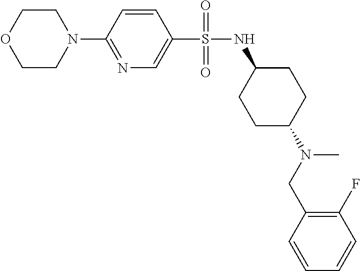 [SMILES: CN(Cc1ccccc1F)[C@H]1CC[C@H](NS(=O)(=O)c2ccc(N3CCOCC3)nc2)CC1]

To a solution of N-((1r,4r)-4-((2-fluorobenzyl)amino)cyclohexyl)-6-morpholinopyridine-3-sulfonamide (example 53) (47 mg, 0.11 mmol) in DCM (4 mL), Na₂SO₄ (19 mg, 0.13 mmol), NaBH(OAc)₃ (178 mg, 0.84 mmol) and formaldehyde (37% in water, 27 μL, 0.37 mmol) were added at 0° C. and the reaction mixture was stirred at rt overnight. The reaction was quenched with ice-cold sat. aq. NaHCO₃ sol, and the product extracted with DCM. The combined organic fractions were washed with water, dried over Na₂SO₄ and the solvent removed under reduced pressure. The crude product was purified by flash chromatography (SiO₂, DCM/MeOH) to give the title compound (20 mg, 41%).

HPLC Rt (Method B):1.98 min; ESI+-MS m/z:463.0 (M+H)⁺.

This method was used for the preparation of Examples 57-60 using suitable starting materials

| | | | Rt | MS | HPLC |
|---|---|---|---|---|---|
| EX | STRUCTURE | CHEMICAL NAME | (min) | (M + H) | Method |
| 57 | 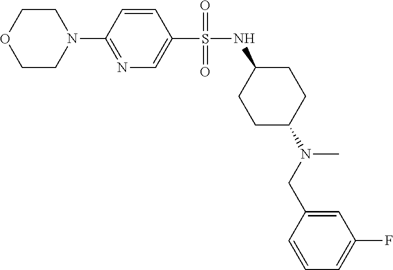 [SMILES: CN(Cc1cccc(F)c1)[C@H]1CC[C@H](NS(=O)(=O)c2ccc(N3CCOCC3)nc2)CC1] | N-((1r,4r)-4-((3-Fluorobenzyl) (methyl)amino)cyclo- hexyl)-6-morpholinopyridine-3- sulfonamide | 2.06 | 463.0 | B |
| 58 | 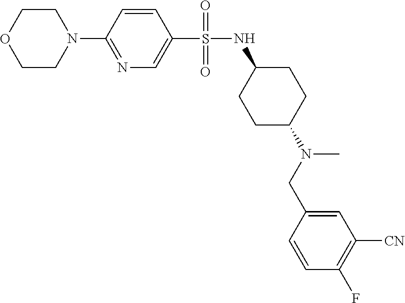 [SMILES: CN(Cc1ccc(F)c(C#N)c1)[C@H]1CC[C@H](NS(=O)(=O)c2ccc(N3CCOCC3)nc2)CC1] | N-((1r,4r)-4-((3-Cyano-4- Fluorobenzyl)(methyl)amino)cyclo- hexyl)-6-morpholinopyridine-3- sulfonamide | 1.98 | 488.2 | C |
| 59 | 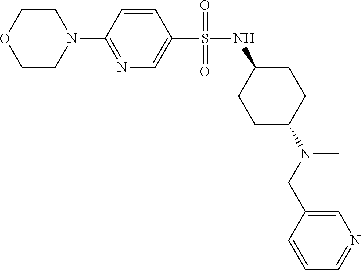 [SMILES: CN(Cc1cccnc1)[C@H]1CC[C@H](NS(=O)(=O)c2ccc(N3CCOCC3)nc2)CC1] | N-((1r,4r)-4-(Methyl(pyridin-3- ylmethyl)amino)cyclohexyl)-6- morpholinopyridine-3-sulfonamide | 1.51 | 446.2 | B |
| 60 | 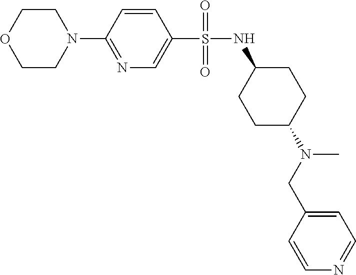 [SMILES: CN(Cc1ccncc1)[C@H]1CC[C@H](NS(=O)(=O)c2ccc(N3CCOCC3)nc2)CC1] | N-((1r,4r)-4-(Methyl(pyridin-4- ylmethyl)amino)cyclohexyl)-6- morpholinopyridine-3-sulfonamide | 1.55 | 446.2 | B |

### Example 61. 4-(5-((9-Benzyl-2,9-diazaspiro[5.5]undecan-2-yl)sulfonyl)pyridin-2-yl)morpholine

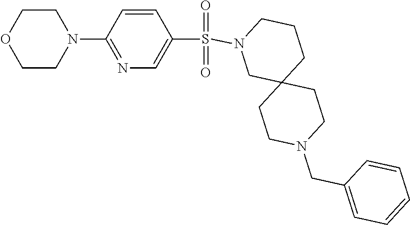 [SMILES: O=S(=O)(c1ccc(N2CCOCC2)nc1)N1CCCC2(CCN(Cc3ccccc3)CC2)C1]

### Step 1. tert-Butyl 2-((6-morpholinopyridin-3-yl)sulfonyl)-2,9-diazaspiro[5.5]undecane-9-carboxylate

Following the procedure described in example 20 using suitable starting material the title compound (quant.) was obtained.

### Step 2. 4-(5-(2,9-Diazaspiro[5.5]undecan-2-ylsulfonyl)pyridin-2-yl)morpholine trifluoroacetate

Starting from compound obtained in step 1 using the procedure described for example 35 step 2, the title compound was obtained quant. as TFA salt.

### Step 3. Title Compound

To the compound obtained in step 2 (46 mg, 0.09 mmol) in ACN (6 mL), K₂CO₃ (64 mg, 0.46 mmol) and benzyl bromide (13 μL, 0.11 mmol) were added. The reaction mixture was stirred at rt overnight. The volatiles were removed under reduced pressure and the residue was partitioned between water and EtOAc. The aqueous phase was additionally extracted with EtOAc and the combined organic fractions were washed with water, dried over Na₂SO₄ and the solvent removed under reduced pressure. The crude product was purified by flash chromatography (SiO₂, DCM/MeOH) to give the title compound (27 mg, 61% yield).

HPLC Rt (Method B): 2.21 min; ESI+-MS m/z: 471.2 (M+H)⁺.

This method was used for the preparation of Example 62 using suitable starting materials.

| | | | Rt | MS | HPLC |
|---|---|---|---|---|---|
| EX | STRUCTURE | CHEMICAL NAME | (min) | (M + H) | Method |
| 62 | 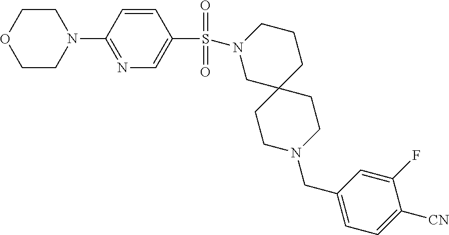 [SMILES: N#Cc1ccc(CN2CCC3(CCCN(S(=O)(=O)c4ccc(N5CCOCC5)nc4)C3)CC2)cc1F] | 2-Fluoro-4-((2-((6- morpholinopyridin- 3-yl)sulfonyl)- 2,9-diazaspiro[5.5] undecan-9- yl)methyl)benzonitrile | 2.28 | 514.3 | C |

### Example 63. 4-(5-((9-(Pyridin-4-ylmethyl)-2,9-diazaspiro[5.5]undecan-2-yl)sulfonyl)pyridin-2-yl)morpholine

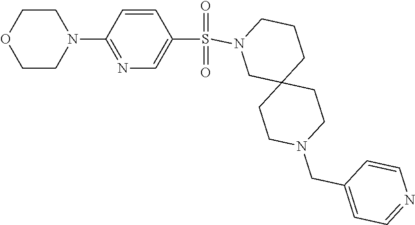 [SMILES: O=S(=O)(c1ccc(N2CCOCC2)nc1)N1CCCC2(CCN(Cc3ccncc3)CC2)C1]

4-(5-(2,9-Diazaspiro[5.5]undecan-2-ylsulfonyl)pyridin-2-yl)morpholine trifluoroacetate (44 mg, 0.09 mmol, prepared as described in example 61, step 2), was dissolved in MeOH (2 mL) and isonicotinaldehyde (17.6 mg, 0.16 mmol) was added followed by NaBH₃CN (13.4 mg, 0.21 mmol). A few drops of AcOH were added and the reaction mixture was stirred at rt overnight. Additional isonicotinaldehyde (21 mg, 0.2 mmol) and NaBH₃CN (15.5 mg, 0.25 mmol) were added and the mixture was stirred for 2 days. The solvent was concentrated and the residue was treated with aqueous NaOH solution (10%). The mixture was extracted with EtOAc and the combined organic fractions were washed with water, dried over Na₂SO₄ and the solvent removed under reduced pressure. The crude product was purified by flash chromatography (SiO₂, DCM/MeOH) to give the title compound (22 mg, 51% yield).

HPLC Rt (Method B): 1.80 min; ESI+-MS m/z: 472.2 (M+H)⁺.

### Example 64. (R)-5-((((1-((6-Morpholinopyridin-3-yl)sulfonyl)piperidin-3-yl)methyl)amino)methyl)pyridin-2-ol

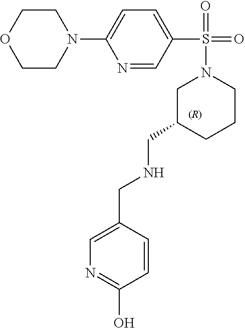

A solution of (R)-1-(6-methoxypyridin-3-yl)-N-((1-((6-morpholinopyridin-3-yl)sulfonyl)piperidin-3-yl)methyl)methanamine (77 mg, 0.17 mmol, obtained in example 33), HBr (48% w/w, 500 μL) and AcOH (0.7 mL) was heated in a sealed tube at 100° C. for 5 h. The reaction was cooled to 0° C. and quenched with aqueous NaOH (10%) sol. The mixture was extracted with EtOAc and the combined organic fractions were washed with water, dried over Na₂SO₄ and the solvent removed under reduced pressure. The crude product was purified by flash chromatography (SiO₂, DCM/MeOH) to give the title compound (36 mg, 48% yield).

HPLC Rt (Method B): 1.33 min; ESI+-MS m/z: 448.2 (M+H)⁺.

This method was used for the preparation of Example 65 using suitable starting materials.

| | | | Rt | MS | HPLC |
|---|---|---|---|---|---|
| EX | STRUCTURE | CHEMICAL NAME | (min) | (M + H) | Method |
| 65 | 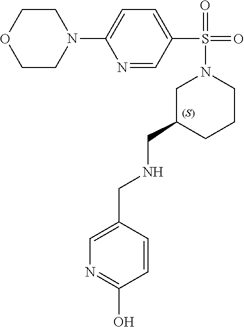 | (S)-5-((((1-((6-Morpholinopyridin- 3-yl)sulfonyl)piperidin-3- yl)methyl)amino)methyl)pyridin-2- ol | 1.33 | 448.2 | B |

### Examples 66 and 67

The following examples were synthesized following the method described in Example 13 using suitable starting materials:

| | | | Rt | MS | |
|---|---|---|---|---|---|
| EX | Structure | Chemical name | (min) | (M + H) | Method |
| 66 | 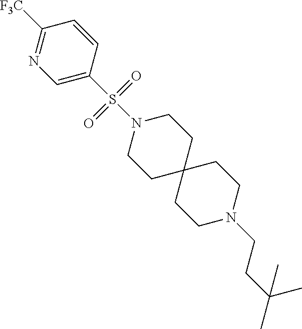 [SMILES: CC(C)(C)CCN1CCC2(CC1)CCN(S(=O)(=O)c1ccc(C(F)(F)F)nc1)CC2] | 3-(3,3-Dimethylbutyl)-9-((6- (trifluoromethyl)pyridin-3- yl)sulfonyl)-3,9- diazaspiro[5.5]undecane | 2.26 | 448.0 | B |
| 67 | 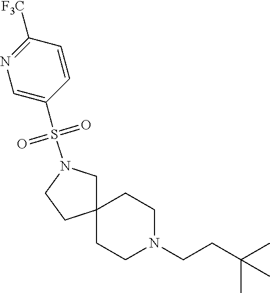 [SMILES: CC(C)(C)CCN1CCC2(CC1)CCN(S(=O)(=O)c1ccc(C(F)(F)F)nc1)C2] | 8-(3,3-Dimethylbutyl)-2-((6- (trifluoromethyl)pyridin-3- yl)sulfonyl)-2,8- diazaspiro[4.5]decane | 2.33 | 434.4 | B |

### Examples 68-87

The following examples were synthesized following the method described in Example 20 using suitable starting materials:

| | | | Rt | MS | |
|---|---|---|---|---|---|
| EX | Structure | Chemical name | (min) | (M + H) | Method |
| 68 | 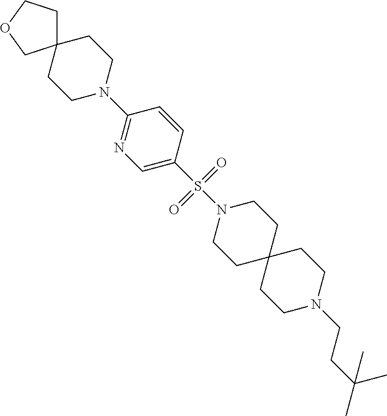 [SMILES: CC(C)(C)CCN1CCC2(CC1)CCN(S(=O)(=O)c1ccc(N3CCC4(CCOC4)CC3)nc1)CC2] | 8-(5-((9-(3,3-Dimethylbutyl)- 3,9-diazaspiro[5.5]undecan-3- yl)sulfonyl)pyridin-2-yl)-2-oxa- 8-azaspiro[4.5]decane | 2.18 | 519.2 | B |
| 69 | 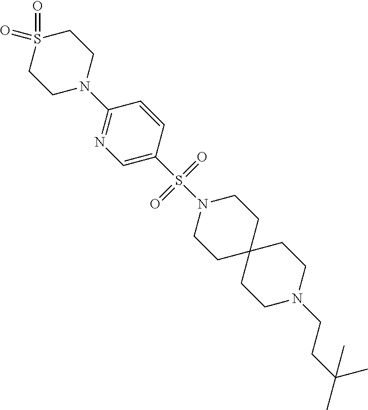 [SMILES: CC(C)(C)CCN1CCC2(CC1)CCN(S(=O)(=O)c1ccc(N3CCS(=O)(=O)CC3)nc1)CC2] | 4-(5-((9-(3,3-Dimethylbutyl)- 3,9-diazaspiro[5.5]undecan-3- yl)sulfonyl)pyridin-2- yl)thiomorpholine 1,1-dioxide | 1.81 | 513.4 | B |
| 70 | 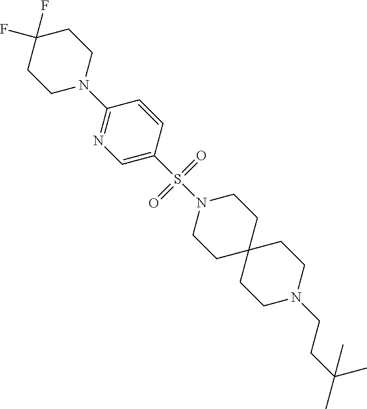 [SMILES: CC(C)(C)CCN1CCC2(CC1)CCN(S(=O)(=O)c1ccc(N3CCC(F)(F)CC3)nc1)CC2] | 3-((6-(4,4-Difluoropiperidin-1- yl)pyridin-3-yl)sulfonyl)-9-(3,3- dimethylbutyl)-3,9- diazaspiro[5.5]undecane | 2.29 | 499.4 | B |
| 71 | 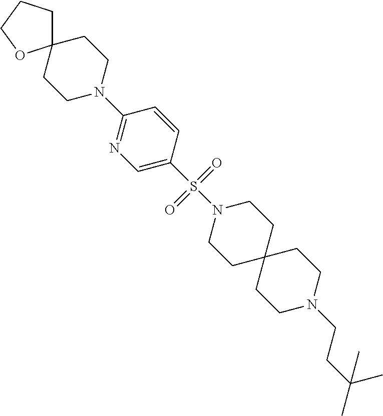 [SMILES: CC(C)(C)CCN1CCC2(CC1)CCN(S(=O)(=O)c1ccc(N3CCC4(CCCO4)CC3)nc1)CC2] | 8-(5-((9-(3,3-Dimethylbutyl)- 3,9-diazaspiro[5.5]undecan-3- yl)sulfonyl)pyridin-2-yl)-1-oxa- 8-azaspiro[4.5]decane | 2.36 | 519.4 | C |
| 72 | 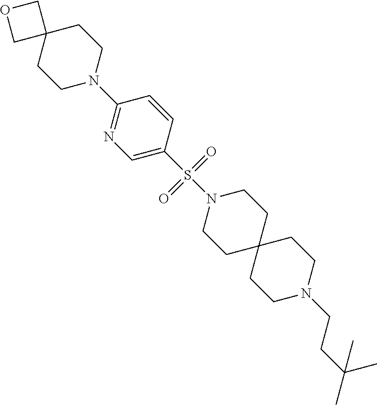 [SMILES: CC(C)(C)CCN1CCC2(CC1)CCN(S(=O)(=O)c1ccc(N3CCC4(CC3)COC4)nc1)CC2] | 7-(5-((9-(3,3-Dimethylbutyl)- 3,9-diazaspiro[5.5]undecan-3- yl)sulfonyl)pyridin-2-yl)-2-oxa- 7-azaspiro[3.5]nonane | 2.04 | 505.3 | C |
| 73 | 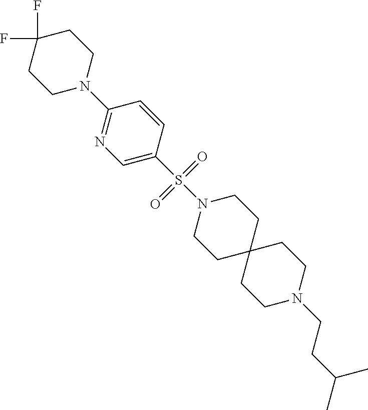 [SMILES: CC(C)CCN1CCC2(CC1)CCN(S(=O)(=O)c1ccc(N3CCC(F)(F)CC3)nc1)CC2] | 3-((6-(4,4-Difluoropiperidin-1- yl)pyridin-3-yl)sulfonyl)-9- isopentyl-3,9- diazaspiro[5.5]undecane | 2.31 | 485.2 | B |
| 74 | 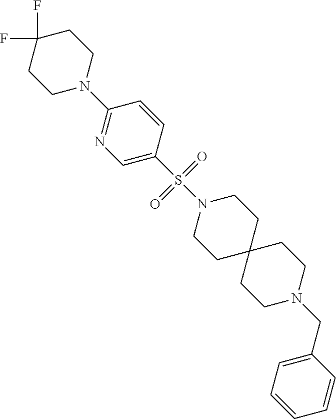 [SMILES: O=S(=O)(c1ccc(N2CCC(F)(F)CC2)nc1)N1CCC2(CCN(Cc3ccccc3)CC2)CC1] | 3-Denzyl-9-((6-(4,4- difluoropiperidin-1-yl)pyridin- 3-yl)sulfonyl)-3,9- diazaspiro[5.5]undecane | 2.54 | 505.2 | B |
| 75 | 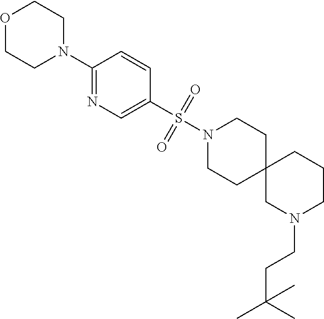 [SMILES: CC(C)(C)CCN1CCCC2(CCN(S(=O)(=O)c3ccc(N4CCOCC4)nc3)CC2)C1] | 4-(5-((2-(3,3-Dimethylbutyl)- 2,9-diazaspiro[5.5]undecan-9- yl)sulfonyl)pyridin-2- yl)morpholine | 2.53 | 465.3 | C |
| 76 | 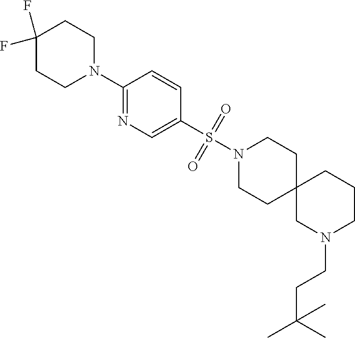 [SMILES: CC(C)(C)CCN1CCCC2(CCN(S(=O)(=O)c3ccc(N4CCC(F)(F)CC4)nc3)CC2)C1] | 9-((6-(4,4-Difluoropiperidin-1- yl)pyridin-3-yl)sulfonyl)-2-(3,3- dimethylbutyl)-2,9- diazaspiro[5.5]undecane | 3.71 | 499.2 | B |
| 77 | 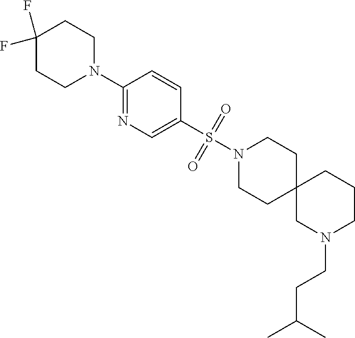 [SMILES: CC(C)CCN1CCCC2(CCN(S(=O)(=O)c3ccc(N4CCC(F)(F)CC4)nc3)CC2)C1] | 9-((6-(4,4-Difluoropiperidin-1- yl)pyridin-3-yl)sulfonyl)-2- isopentyl-2,9- diazaspiro[5.5]undecane | 2.34 | 485.2 | B |
| 78 | 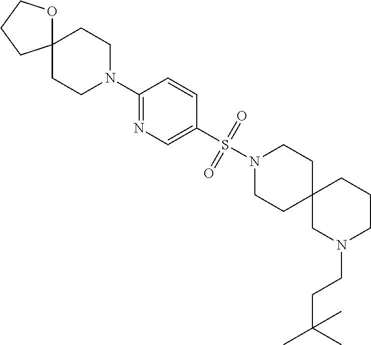 [SMILES: CC(C)(C)CCN1CCCC2(CCN(S(=O)(=O)c3ccc(N4CCC5(CCCO5)CC4)nc3)CC2)C1] | 8-(5-((2-(3,3-Dimethylbutyl)- 2,9-diazaspiro[5.5]undecan-9- yl)sulfonyl)pyridin-2-yl)-1-oxa- 8-azaspiro[4.5]decane | 3.03 | 519.2 | B |
| 79 | 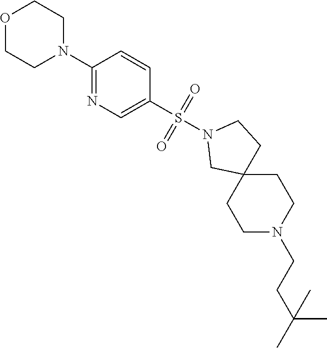 [SMILES: CC(C)(C)CCN1CCC2(CC1)CCN(S(=O)(=O)c1ccc(N3CCOCC3)nc1)C2] | 4-(5-((8-(3,3-Dimethylbutyl)- 2,8-diazaspiro[4.5]decan-2- yl)sulfonyl)pyridin-2- yl)morpholine | 2.02 | 451.2 | B |
| 80 | 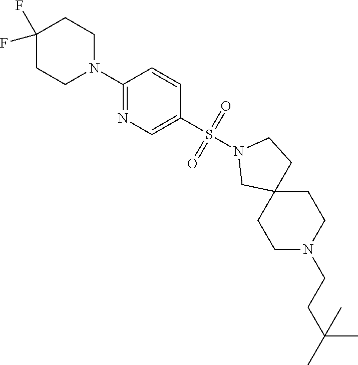 [SMILES: CC(C)(C)CCN1CCC2(CC1)CCN(S(=O)(=O)c1ccc(N3CCC(F)(F)CC3)nc1)C2] | 2-((6-(4,4-Difluoropiperidin-1- yl)pyridin-3-yl)sulfonyl)-8-(3,3- dimethylbutyl)-2,8- diazaspiro[4.5]decane | 2.42 | 485.2 | B |
| 81 | 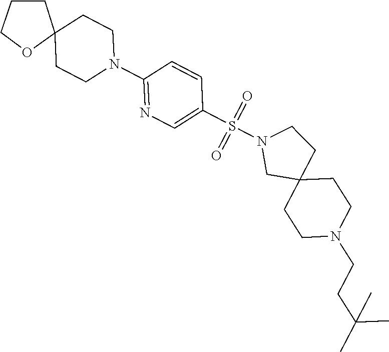 [SMILES: CC(C)(C)CCN1CCC2(CC1)CCN(S(=O)(=O)c1ccc(N3CCC4(CCCO4)CC3)nc1)C2] | 8-(5-((8-(3,3-Dimethylbutyl)- 2,8-diazaspiro[4.5]decan-2- yl)sulfonyl)pyridin-2-yl)-1-oxa- 8-azaspiro[4.5]decane | 2.44 | 505.2 | B |
| 82 | 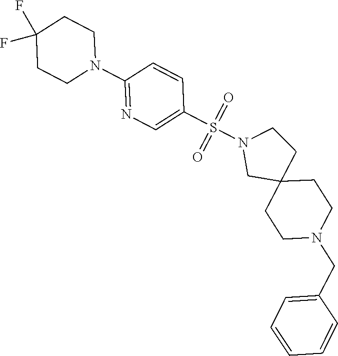 [SMILES: O=S(=O)(c1ccc(N2CCC(F)(F)CC2)nc1)N1CCC2(CCN(Cc3ccccc3)CC2)C1] | 8-Benzyl-2-((6-(4,4- difluoropiperidin-1-yl)pyridin- 3-yl)sulfonyl)-2,8- diazaspiro[4.5]decane | 2.42 | 491.2 | B |
| 83 | 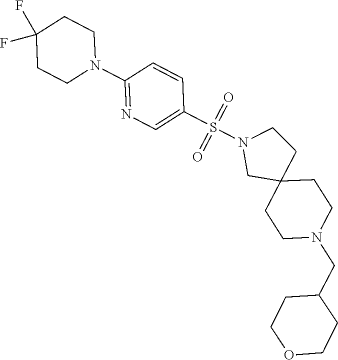 [SMILES: O=S(=O)(c1ccc(N2CCC(F)(F)CC2)nc1)N1CCC2(CCN(CC3CCOCC3)CC2)C1] | 2-((6-(4,4-Difluoropiperidin-1- yl)pyridin-3-yl)sulfonyl)-8- ((tetrahydro-2H-pyran-4- yl)methyl)-2,8- diazaspiro[4.5]decane | 2.04 | 499.2 | B |
| 84 | 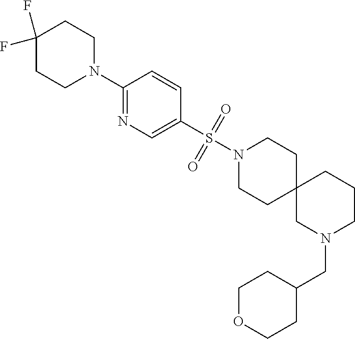 [SMILES: O=S(=O)(c1ccc(N2CCC(F)(F)CC2)nc1)N1CCC2(CCCN(CC3CCOCC3)C2)CC1] | 9-((6-(4,4-Difluoropiperidin-1- yl)pyridin-3-yl)sulfonyl)-2- ((tetrahydro-2H-pyran-4- yl)methyl)-2,9- diazaspiro[5.5]undecane | 2.58 | 513.2 | B |
| 85 | 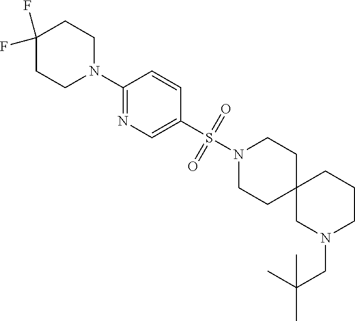 [SMILES: CC(C)(C)CN1CCCC2(CCN(S(=O)(=O)c3ccc(N4CCC(F)(F)CC4)nc3)CC2)C1] | 9-((6-(4,4-Difluoropiperidin-1- yl)pyridin-3-yl)sulfonyl)-2- neopentyl-2,9- diazaspiro[5.5]undecane | 3.04 | 485.2 | B |
| 86 | 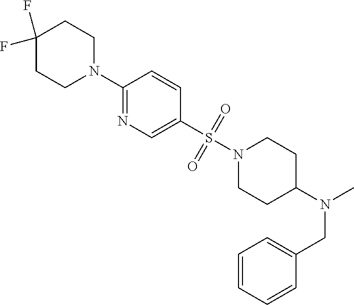 [SMILES: CN(Cc1ccccc1)C1CCN(S(=O)(=O)c2ccc(N3CCC(F)(F)CC3)nc2)CC1] | N-Benzyl-1-((6-(4,4- difluoropiperidin-1-yl)pyridin- 3-yl)sulfonyl)-N- methylpiperidin-4-amine | 2.46 | 465.0 | B |
| 87 | 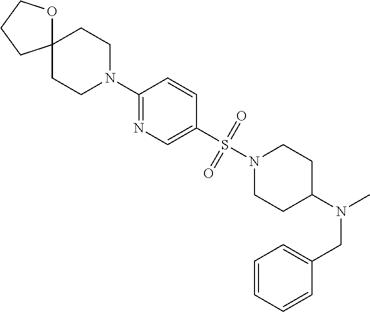 [SMILES: CN(Cc1ccccc1)C1CCN(S(=O)(=O)c2ccc(N3CCC4(CCCO4)CC3)nc2)CC1] | 1-((6-(1-Oxa-8- azaspiro[4.5]decan-8- yl)pyridin-3-yl)sulfonyl)-N- benzyl-N-methylpiperidin-4- amine | 2.51 | 485.2 | B |

### Example 88

The following example was synthesized following the method described in Example 35 using suitable starting materials:

| | | | Rt | MS | |
|---|---|---|---|---|---|
| EX | Structure | Chemical name | (min) | (M + H) | Method |
| 88 | 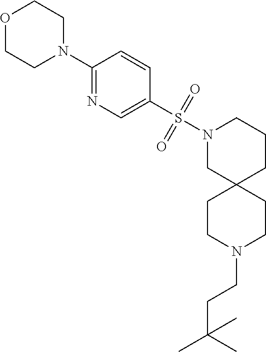 [SMILES: CC(C)(C)CCN1CCC2(CCCN(S(=O)(=O)c3ccc(N4CCOCC4)nc3)C2)CC1] | 4-(5-((9-(3,3-Dimethylbutyl)-2,9- diazaspiro[5.5]undecan-2- yl)sulfonyl)pyridin-2-yl)morpholine | 2.08 | 465.2 | B |

### Example 89

The following example was synthesized following the method described in Example 56 using suitable starting materials:

| | | | Rt | MS | |
|---|---|---|---|---|---|
| EX | Structure | Chemical name | (min) | (M + H) | Method |
| 89 | 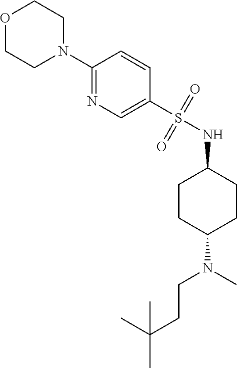 [SMILES: CN(CCC(C)(C)C)[C@H]1CC[C@H](NS(=O)(=O)c2ccc(N3CCOCC3)nc2)CC1] | N-((1r,4r)-4-((3,3- Dimethylbutyl)(methyl)amino)cyclo- hexyl)-6-morpholinopyridine-3- sulfonamide | 1.67 | 439.2 | B |

### Example 90

The following example was synthesized following the method described in Example 61 using suitable starting materials:

| | | | Rt | MS | |
|---|---|---|---|---|---|
| EX | Structure | Chemical name | (min) | (M + H) | Method |
| 90 | 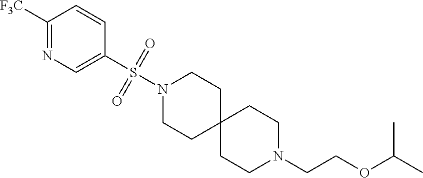 [SMILES: CC(C)OCCN1CCC2(CC1)CCN(S(=O)(=O)c1ccc(C(F)(F)F)nc1)CC2] | 3-(2-Isopropoxyethyl)-9-((6- (trifluoromethyl)pyridin-3- yl)sulfonyl)-3,9- diazaspiro[5.5]undecane | 2.07 | 450.2 | B |

### Examples 91-93

The following examples were synthesized following the method described in Example 63 using suitable starting materials:

| | | | Rt | MS | |
|---|---|---|---|---|---|
| EX | Structure | Chemical name | (min) | (M + H) | Method |
| 91 | 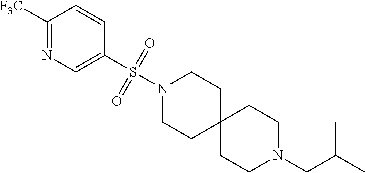 [SMILES: CC(C)CN1CCC2(CC1)CCN(S(=O)(=O)c1ccc(C(F)(F)F)nc1)CC2] | 3-Isobutyl-9-((6- (trifluoromethyl)pyridin-3- yl)sulfonyl)-3,9- diazaspiro[5.5]undecane | 2.30 | 420.2 | B |
| 92 | 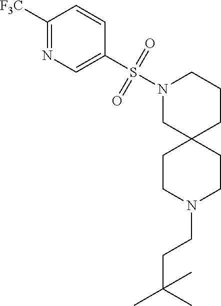 [SMILES: CC(C)(C)CCN1CCC2(CCCN(S(=O)(=O)c3ccc(C(F)(F)F)nc3)C2)CC1] | 9-(3,3-Dimethylbutyl)-2-((6- (trifluoromethyl)pyridin-3- yl)sulfonyl)-2,9- diazaspiro[5.5]undecane | 2.31 | 448.0 | B |
| 93 | 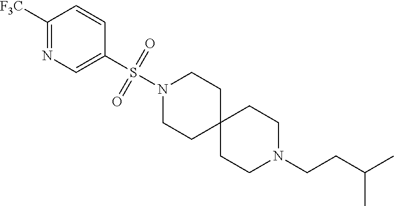 [SMILES: CC(C)CCN1CCC2(CC1)CCN(S(=O)(=O)c1ccc(C(F)(F)F)nc1)CC2] | 3-Isopentyl-9-((6- (trifluoromethyl)pyridin-3- yl)sulfonyl)-3,9- diazaspiro[5.5]undecane | 2.52 | 434.4 | B |

### Example 94. 5-((9-(3,3-Di methyl butyl)-2,9-diazaspiro[5.5]undecan-2-yl)sulfonyl)-N-methylpyridin-2-amine

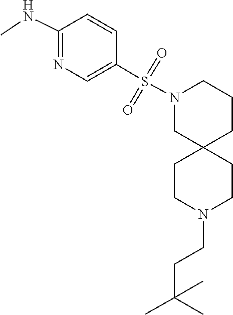 [SMILES: CNc1ccc(S(=O)(=O)N2CCCC3(CCN(CCC(C)(C)C)CC3)C2)cn1]

### Step 1. tert-Butyl 2-((6-chloropyridin-3-yl)sulfonyl)-2,9-diazaspiro[5.5]undecane-9-carboxylate

Starting from 6-chloropyridine-3-sulfonyl chloride (460 mg, 2.15 mmol) and tert-butyl 2,9-diazaspiro[5.5]undecane-9-carboxylate (607 mg, 2.4 mmol) and following the experimental procedure described in example 13, the title compound was obtained (1.05 g, Quant.).

HPLC Rt (Method B): 2.41 min; ESI+-MS m/z: 374.0 (M+H)⁺.

### Step 2. 2-((6-Chloropyridin-3-yl)sulfonyl)-2,9-diazaspiro[5.5]undecane hydrochloride

Starting from the compound obtained in step 1 (1.05 g, 2.15 mmol) and following a similar procedure to that described in step 2b of intermediate 4, the title compound was obtained (878 mg, Quant.).

HPLC Rt (Method B): 1.33 min; ESI+-MS m/z: 330.0 (M+H)⁺.

### Step 3. 2-((6-Chloropyridin-3-yl)sulfonyl)-9-(3,3-dimethylbutyl)-2,9-diazaspiro[5.5]undecane

Starting from the compound obtained in step 2 (878 mg, 2.4 mmol) and following a similar procedure to that described in step 1 of intermediate 4, the title compound was obtained (759 mg, 76% yield).

HPLC Rt (Method B): 2.26 min; ESI+-MS m/z: 414.0 (M+H)⁺.

### Step 4. Title Compound

Starting from the compound obtained in step 3 (100 mg, 0.24 mmol) and following a similar procedure to that described in step 2 of example 20, the title compound was obtained (71 mg, 68% yield).

HPLC Rt (Method B): 1.79 min; ESI+-MS m/z: 409.2 (M+H)⁺.

This method was used for the preparation of Examples 95-100 using suitable starting materials:

| | | | Rt | MS | |
|---|---|---|---|---|---|
| EX | Structure | Chemical name | (min) | (M + H) | Method |
| 95 | 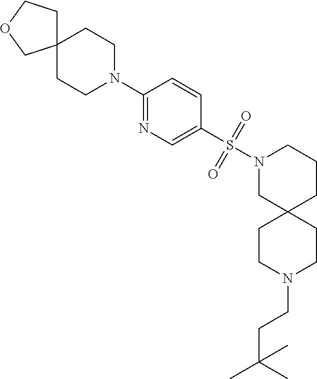 [SMILES: CC(C)(C)CCN1CCC2(CCCN(S(=O)(=O)c3ccc(N4CCC5(CCOC5)CC4)nc3)C2)CC1] | 8-(5-((9-(3,3-Dimethylbutyl)- 2,9-diazaspiro[5.5]undecan-2- yl)sulfonyl)pyridin-2-yl)-2-oxa- 8-azaspiro[4.5]decane | 3.86 | 519.2 | B |
| 96 | 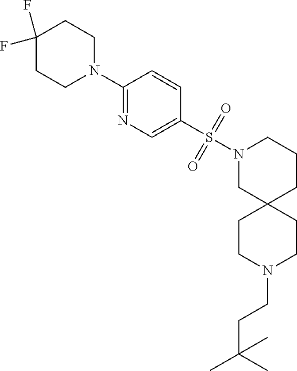 [SMILES: CC(C)(C)CCN1CCC2(CCCN(S(=O)(=O)c3ccc(N4CCC(F)(F)CC4)nc3)C2)CC1] | 2-((6-(4,4-Difluoropiperidin-1- yl)pyridin-3-yl)sulfonyl)-9-(3,3- dimethylbutyl)-2,9- diazaspiro[5.5]undecane | 2.49 | 499.3 | C |
| 97 | 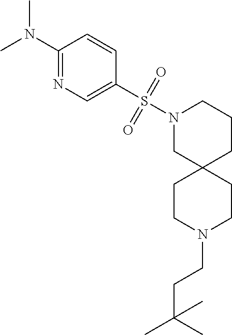 [SMILES: CN(C)c1ccc(S(=O)(=O)N2CCCC3(CCN(CCC(C)(C)C)CC3)C2)cn1] | 5-((9-(3,3-Dimethylbutyl)-2,9- diazaspiro[5.5]undecan-2- yl)sulfonyl)-N,N- dimethylpyridin-2-amine | 2.13 | 423.3 | C |
| 98 | 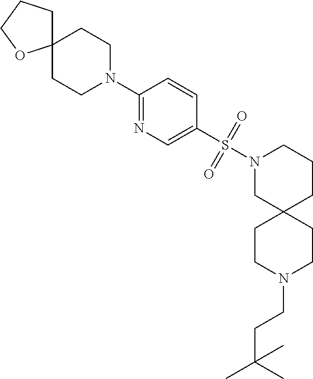 [SMILES: CC(C)(C)CCN1CCC2(CCCN(S(=O)(=O)c3ccc(N4CCC5(CCCO5)CC4)nc3)C2)CC1] | 8-(5-((9-(3,3-Dimethylbutyl)- 2,9-diazaspiro[5.5]undecan-2- yl)sulfonyl)pyridin-2-yl)-1-oxa- 8-azaspiro[4.5]decane | 2.54 | 519.4 | C |
| 99 | 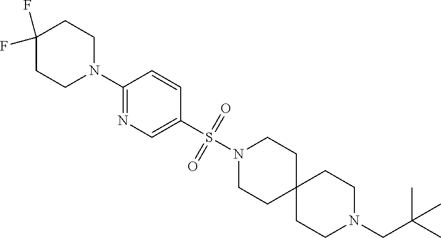 [SMILES: CC(C)(C)CN1CCC2(CC1)CCN(S(=O)(=O)c1ccc(N3CCC(F)(F)CC3)nc1)CC2] | 3-((6-(4,4-Difluoropiperidin-1- yl)pyridin-3-yl)sulfonyl)-9- neopentyl-3,9- diazaspiro[5.5]undecane | 2.93 | 485.2 | B |
| 100 | 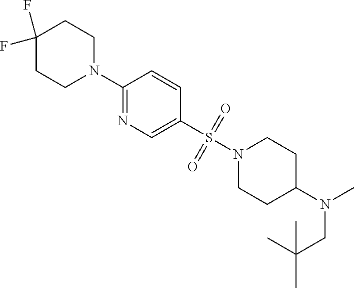 [SMILES: CN(CC(C)(C)C)C1CCN(S(=O)(=O)c2ccc(N3CCC(F)(F)CC3)nc2)CC1] | 1-((6-(4,4-Difluoropiperidin-1- yl)pyridin-3-yl)sulfonyl)-N- methyl-N-neopentylpiperidin- 4-amine | 2.73 | 445.2 | B |
| 101 | 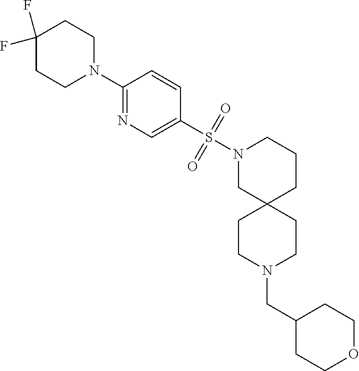 [SMILES: O=S(=O)(c1ccc(N2CCC(F)(F)CC2)nc1)N1CCCC2(CCN(CC3CCOCC3)CC2)C1] | 2-((6-(4,4-Difluoropiperidin-1- yl)pyridin-3-yl)sulfonyl)-9- ((tetrahydro-2H-pyran-4- yl)methyl)-2,9- diazaspiro[5.5]undecane | 2.17 | 513.2 | C |

### Example 102. N-(5-((9-(3,3-Dimethylbutyl)-2,9-diazaspiro[5.5]undecan-2-yl)sulfonyl)pyridin-2-yl)-N-methylacetamide

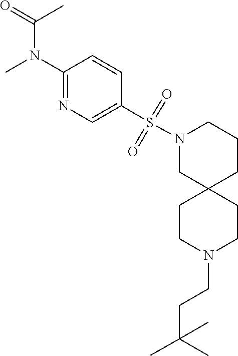 [SMILES: CC(=O)N(C)c1ccc(S(=O)(=O)N2CCCC3(CCN(CCC(C)(C)C)CC3)C2)cn1]

A mixture of the compound obtained in example 94 (63 mg, 0.15 mmol) and acetic anhydride (0.36 mL, 3.8 mmol) was placed in a microwave vial. The system was purged with N₂ and it was irradiated under microwave heating at 120° C. for 20 min. After cooling down to rt, the reaction was quenched with ice. The residue was dissolved in EtOAc and it was washed with aq. sat. NaHCO₃ sol. The aqueous phase was further extracted with EtOAc. The combined organic extracts were washed with water, dried over Na₂SO₄, filtered and concentrated to dryness. The residue was purified by flash chromatography (DCM:MeOH) to give the title compound (18 mg, 26% yield).

HPLC Rt (Method B): 1.89 min; ESI+-MS m/z: 451.2 (M+H)⁺.

This method was used for the preparation of Examples 103-105 using suitable starting materials:

| | | | Rt | MS | HPLC |
|---|---|---|---|---|---|
| EX | STRUCTURE | CHEMICAL NAME | (min) | (M + H) | Method |
| 103 | 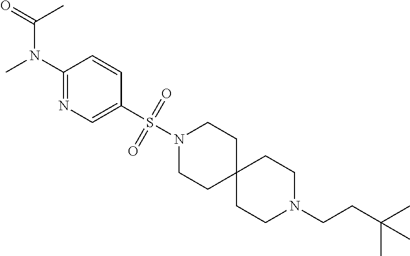 [SMILES: CC(=O)N(C)c1ccc(S(=O)(=O)N2CCC3(CCN(CCC(C)(C)C)CC3)CC2)cn1] | N-(5-((9-(3,3-Dimethylbutyl)-3,9- diazaspiro[5.5]undecan-3- yl)sulfonyl)pyridin-2-yl)-N- methylacetamide | 1.76 | 451.2 | B |
| 104 | 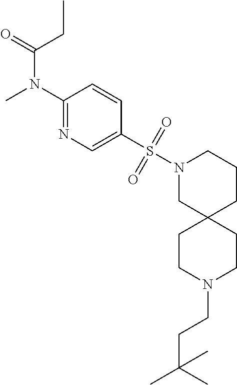 [SMILES: CCC(=O)N(C)c1ccc(S(=O)(=O)N2CCCC3(CCN(CCC(C)(C)C)CC3)C2)cn1] | N-(5-((9-(3,3-Dimethylbutyl)-2,9- diazaspiro[5.5]undecan-2- yl)sulfonyl)pyridin-2-yl)-N- methylpropionamide | 2.11 | 465.3 | C |
| 105 | 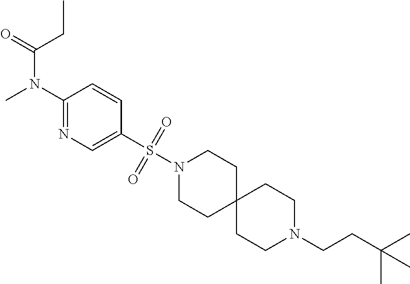 [SMILES: CCC(=O)N(C)c1ccc(S(=O)(=O)N2CCC3(CCN(CCC(C)(C)C)CC3)CC2)cn1] | N-(5-((9-(3,3-Dimethylbutyl)-3,9- diazaspiro[5.5]undecan-3- yl)sulfonyl)pyridin-2-yl)-N- methylpropionamide | 1.99 | 465.2 | B |

### Example 106. N-(5-((9-(3,3-Dimethylbutyl)-2,9-diazaspiro[5.5]undecan-2-yl)sulfonyl)pyridin-2-yl)propionamide

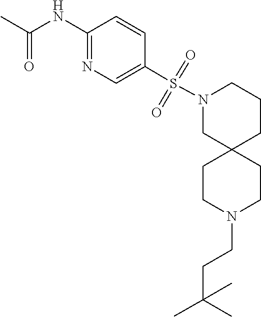 [SMILES: CC(=O)Nc1ccc(S(=O)(=O)N2CCCC3(CCN(CCC(C)(C)C)CC3)C2)cn1]

A sealed tube charged with the compound obtained in step 3 of Example 94 (89 mg, 0.21 mmol), propionamide (31 mg, 0.43 mmol), Pd₂(dba)₃ (3 mg, 0.003 mmol), XantPhos (6 mg, 0.011 mmol) and K₃PO₄ (55 mg, 0.26 mmol), was evacuated and backfilled with argon. Dioxane (2 mL), degassed by means of bubbling argon to the solution for 5 min, was added and the reaction mixture was irradiated under microwave heating at 150° C. for 2 h. The resulting mixture was filtered and the solvent was removed under vacuum. The crude product was purified by flash chromatography (DCM:MeOH) to give the title compound (24 mg, 24% yield).

HPLC Rt (Method C): 2.00 min; ESI+-MS m/z: 451.3 (M+H)⁺.

This method was used for the preparation of Example 107 using suitable starting materials:

| | | | Rt | MS | HPLC |
|---|---|---|---|---|---|
| EX | STRUCTURE | CHEMICAL NAME | (min) | (M + H) | Method |
| 107 | 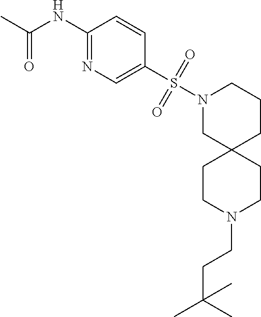 [SMILES: CC(=O)Nc1ccc(S(=O)(=O)N2CCCC3(CCN(CCC(C)(C)C)CC3)C2)cn1] | N-(5-((9-(3,3-Dimethylbutyl)-2,9- diazaspiro[5.5]undecan-2- yl)sulfonyl)pyridin-2-yl)acetamide | 1.87 | 437.3 | C |

### Example 108. 9-(3,3-Dimethylbutyl)-2-((6-methylpyridin-3-yl)sulfonyl)-2,9-diazaspiro[5.5]undecane

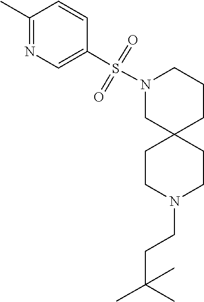 [SMILES: Cc1ccc(S(=O)(=O)N2CCCC3(CCN(CCC(C)(C)C)CC3)C2)cn1]

A sealed tube charged with the compound obtained in step 3 of Example 94 (100 mg, 0.24 mmol), a 2 M dimethylzinc solution in toluene (0.13 mL, 0.24 mmol) and Ni(dppp)Cl₂ (13 mg, 0.024 mmol) and THE (5 mL) was degassed by means of bubbling argon to the solution for 5 min and the reaction mixture was stirred at rt for 16 h. The solvent was removed under vacuum, the residue was dissolved in EtOAc and washed with aq. sat. NaHCO₃ sol. The aqueous phase was further extracted with EtOAc and the combined organic layers were dried over Na₂SO₄, filtered, and concentrated under vacuum. The crude product was purified by flash chromatography (Chx:EtOAc) to give the title compound (55 mg, 58% yield).

HPLC Rt (Method B): 1.90 min; ESI+-MS m/z: 394.2 (M+H)⁺.

This method was used for the preparation of Examples 109-111 using suitable starting materials:

| | | | Rt | MS | HPLC |
|---|---|---|---|---|---|
| EX | STRUCTURE | CHEMICAL NAME | (min) | (M + H) | Method |
| 109 | 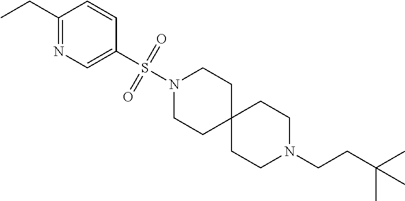 [SMILES: CCc1ccc(S(=O)(=O)N2CCC3(CCN(CCC(C)(C)C)CC3)CC2)cn1] | 3-(3,3-Dimethylbutyl)-9-((6- ethylpyridin-3-yl)sulfonyl)- 3,9-diazaspiro[5.5]undecane | 2.08 | 408.2 | B |
| 110 | 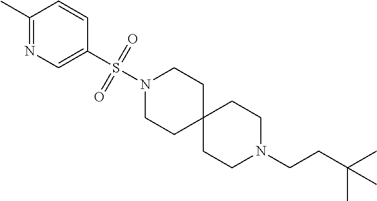 [SMILES: Cc1ccc(S(=O)(=O)N2CCC3(CCN(CCC(C)(C)C)CC3)CC2)cn1] | 3-(3,3-Dimethylbutyl)-9- ((6-methylpyridin-3- yl)sulfonyl)-3,9- diazaspiro[5.5]undecane | 1.89 | 394.2 | B |
| 111 | 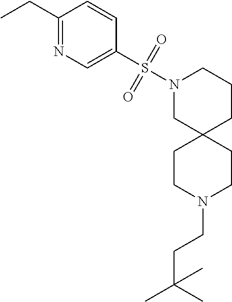 [SMILES: CCc1ccc(S(=O)(=O)N2CCCC3(CCN(CCC(C)(C)C)CC3)C2)cn1] | 9-(3,3-Dimethylbutyl)-2-((6- ethylpyridin-3-yl)sulfonyl)- 2,9-diazaspiro[5.5]undecane | 2.08 | 408.2 | B |

### Example 112. 2-([2,4′-Bipyridin]-5-ylsulfonyl)-9-(3,3-dimethylbutyl)-2,9-diazaspiro[5.5]undecane

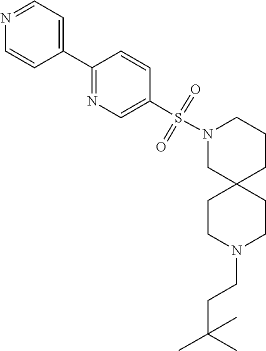 [SMILES: CC(C)(C)CCN1CCC2(CCCN(S(=O)(=O)c3ccc(-c4ccncc4)nc3)C2)CC1]

A sealed tube charged with the compound obtained in step 3 of Example 94 (50 mg, 0.12 mmol), pyridin-4-ylboronic acid (21 mg, 0.17 mmol), Pd(dppf)Cl₂ (7 mg, 0.012 mmol) and Na₂CO₃ (41 mg, 0.38 mmol), was evacuated and backfilled with argon. Dioxane:H₂O (3:1, 1.3 mL), degassed by means of bubbling argon to the solution for 5 min, was added and the reaction mixture was stirred at 130° C. for 1 h. The solvent was removed under vacuum, the residue was dissolved in EtOAc and washed with 10% aq. KOH sol. The aqueous phase was further extracted with EtOAc and the combined organic layers were dried over Na₂SO₄, filtered, and concentrated under vacuum. The crude product was purified by flash chromatography (DCM:MeOH) to give the title compound (20 mg, 36% yield).

HPLC Rt (Method B): 2.12 min; ESI+-MS m/z: 457.2 (M+H)⁺.

This method was used for the preparation of Examples 113-115 using suitable starting materials:

| | | | Rt | MS | HPLC |
|---|---|---|---|---|---|
| EX | STRUCTURE | CHEMICAL NAME | (min) | (M + H) | Method |
| 113 | 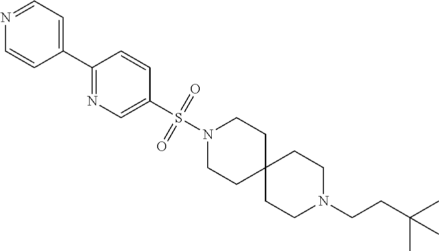 [SMILES: CC(C)(C)CCN1CCC2(CC1)CCN(S(=O)(=O)c1ccc(-c3ccncc3)nc1)CC2] | 3-([2,4′-Bipyridin]-5- ylsulfonyl)-9-(3,3- dimethylbutyl)-3,9- diazaspiro[5.5]undecane | 2.82 | 457.2 | B |
| 114 | 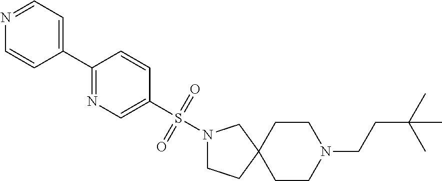 [SMILES: CC(C)(C)CCN1CCC2(CC1)CCN(S(=O)(=O)c1ccc(-c3ccncc3)nc1)C2] | 2-([2,4′-Bipyridin]-5- ylsulfonyl)-8-(3,3- dimethylbutyl)-2,8- diazaspiro[4.5]decane | 2.10 | 443.2 | B |
| 115 | 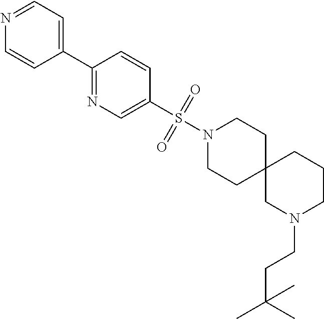 [SMILES: CC(C)(C)CCN1CCCC2(CCN(S(=O)(=O)c3ccc(-c4ccncc4)nc3)CC2)C1] | 9-([2,4′-Bipyridin]-5- ylsulfonyl)-2-(3,3- dimethylbutyl)-2,9- diazaspiro[5.5]undecane | 2.54 | 457.2 | B |

### Example 116. 9-(3,3-Dimethylbutyl)-2-((6-(tetrahydro-2H-pyran-4-yl)pyridin-3-yl)sulfonyl)-2,9-diazaspiro[5.5]undecane

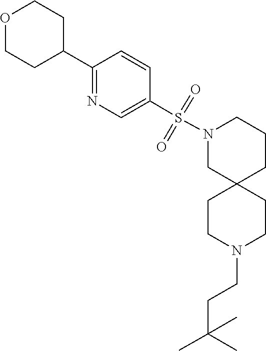 [SMILES: CC(C)(C)CCN1CCC2(CCCN(S(=O)(=O)c3ccc(C4CCOCC4)nc3)C2)CC1]

### Step 1. 2-((6-(3,6-Dihydro-2H-pyran-4-yl)pyridin-3-yl)sulfonyl)-9-(3,3-dimethylbutyl)-2,9-diazaspiro[5.5]undecane

A sealed tube charged with the compound obtained in step 3 of Example 94 (60 mg, 0.14 mmol), 2-(3,6-dihydro-2H-pyran-4-yl)-4,4,5,5-tetramethyl-1,3,2-dioxaborolane (46 mg, 0.22 mmol), Pd(PPh₃)₄ (17 mg, 0.014 mmol) and Na₂CO₃ (46 mg, 0.43 mmol), was evacuated and backfilled with argon. Dioxane:H₂O (3:1, 3 mL), degassed by means of bubbling argon to the solution for 5 min, was added and the reaction mixture was stirred at 90° C. for 16 h. The solvent was removed under vacuum, the residue was dissolved in EtOAc and washed with aq. sat. NaHCO₃ sol. The aqueous phase was further extracted with EtOAc and the combined organic layers were dried over Na₂SO₄, filtered and concentrated under vacuum to give the title compound (92 mg, Quant).

HPLC Rt (Method B): 2.15 min; ESI+-MS m/z: 462.4 (M+H)⁺.

### Step 2. Title Compound

A solution of the compound obtained in step 1 (30 mg, 0.065 mmol) in MeOH (3 mL) was purged with N₂ in a sealed tube. Palladium (7 mg, 10% wt. on charcoal) was added. The tube was purged with H₂ and the reaction mixture was stirred at r.t. overnight. The catalyst was filtered off and the solvent was evaporated. The crude product was purified by flash chromatography (DCM:MeOH) to give the title compound (29 mg, 99% yield).

HPLC Rt (Method C): 2.04 min; ESI+-MS m/z: 464.3 (M+H)⁺.

This method was used for the preparation of Example 117 using suitable starting materials:

| | | | Rt | MS | HPLC |
|---|---|---|---|---|---|
| EX | STRUCTURE | CHEMICAL NAME | (min) | (M + H) | Method |
| 117 | 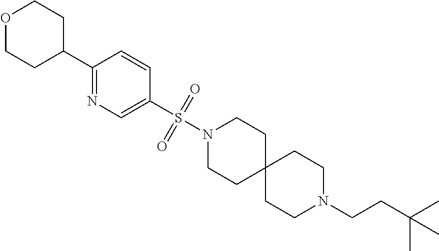 [SMILES: CC(C)(C)CCN1CCC2(CC1)CCN(S(=O)(=O)c1ccc(C3CCOCC3)nc1)CC2] | 3-(3,3-Dimethylbutyl)-9-((6- (tetrahydro-2H-pyran-4- yl)pyridin-3-yl)sulfonyl)-3,9- diazaspiro[5.5]undecane | 1.99 | 464.4 | B |

### Example 118. 4-(5-((9-(3,3-Dimethylbutyl)-2,9-diazaspiro[5.5]undecan-2-yl)sulfonyl)pyridin-2-yl)thiomorpholine 1,1-dioxide

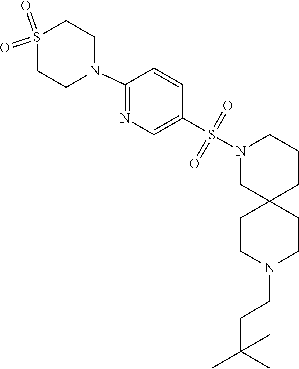 [SMILES: CC(C)(C)CCN1CCC2(CCCN(S(=O)(=O)c3ccc(N4CCS(=O)(=O)CC4)nc3)C2)CC1]

A solution of the compound obtained in step 3 of Example 94 (75 mg, 0.18 mmol), thiomorpholine 1,1-dioxide hydrochloride (62 mg, 0.36 mmol), Pd₂(dba)₃ (24 mg, 0.036 mmol), DavePhos (43 mg, 0.11 mmol) and K₃PO₄ (192 mg, 0.9 mmol) in dioxane under Ar atmosphere was heated at 80° C. for 16 h. After this time, solvent was removed under reduced pressure and the residue was diluted with EtOAc and washed with aq. sat. NaHCO₃ sol. The organic layer was dried over anh Na₂SO₄, filtered and concentrated to dryness. The crude product was purified by flash chromatography, silica gel, DCM:MeOH to give the title compound (21 mg, 22% yield).

HPLC-MS Rt (Method C): 1.89 min; ESI⁺-MS m/z: 513.2 (M+1).

This method was used for the preparation of Example 119 using suitable starting materials:

| | | | Rt | MS | HPLC |
|---|---|---|---|---|---|
| EX | STRUCTURE | CHEMICAL NAME | (min) | (M + H) | Method |
| 119 | 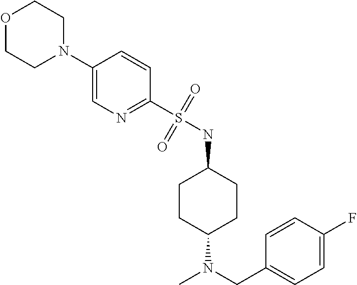 [SMILES: CN(Cc1ccc(F)cc1)[C@H]1CC[C@H](NS(=O)(=O)c2ccc(N3CCOCC3)cn2)CC1] | N-((1r,4r)-4-((4-Fluoro- benzyl)(methyl)amino)cyclo- hexyl)-5-morpholinopyridine- 2-sulfonamide | 1.90 | 463.0 | B |

### Example 120. 4-(5-((9-(3,3-Dimethylbutyl)-2,9-diazaspiro[5.5]undecan-2-yl)sulfonyl)pyridin-2-yl)morpholin-3-one

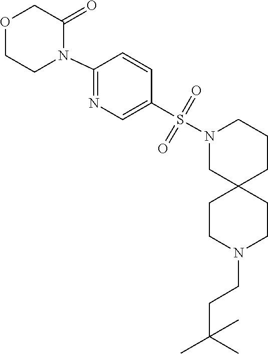 [SMILES: CC(C)(C)CCN1CCC2(CCCN(S(=O)(=O)c3ccc(N4CCOCC4=O)nc3)C2)CC1]

A sealed tube charged with the compound obtained in the step 3 of Example 94 (75 mg, 0.18 mmol), morpholin-3-one (22 mg, 0.22 mmol), Pd(OAc)₂ (4 mg, 0.018 mmol), XantPhos (16 mg, 0.027 mmol) and C₅₂CO₃ (83 mg, 0.25 mmol), was evacuated and backfilled with argon. Dioxane (2 mL), degassed by means of bubbling argon to the solution for 5 min, was added and the reaction mixture was stirred at 110° C. overnight. The solvent was removed under vacuum, the residue was dissolved in EtOAc and washed with aq. sat. NaHCO₃ sol. The aqueous phase was further extracted with EtOAc and the combined organic layers were dried over Na₂SO₄, filtered, and concentrated under vacuum. The crude product was purified by flash chromatography (DCM:MeOH) to give the title compound (47 mg, 50% yield).

HPLC Rt (Method C): 1.97 min; ESI+-MS m/z: 479.3 (M+H)⁺.

This method was used for the preparation of Examples 121-127 using suitable starting materials:

| | | | Rt | MS | HPLC |
|---|---|---|---|---|---|
| EX | STRUCTURE | CHEMICAL NAME | (min) | (M + H) | Method |
| 121 | 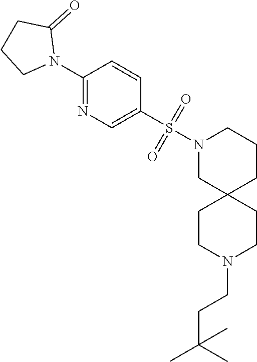 [SMILES: CC(C)(C)CCN1CCC2(CCCN(S(=O)(=O)c3ccc(N4CCCC4=O)nc3)C2)CC1] | 1-(5-((9-(3,3-Dimethylbutyl)- 2,9-diazaspiro[5.5]undecan-2- yl)sulfonyl)pyridin-2- yl)pyrrolidin-2-one | 2.12 | 463.3 | C |
| 122 | 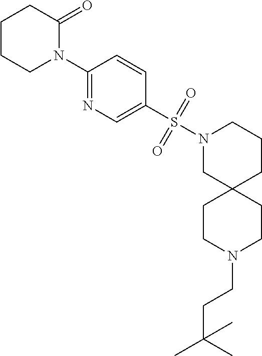 [SMILES: CC(C)(C)CCN1CCC2(CCCN(S(=O)(=O)c3ccc(N4CCCCC4=O)nc3)C2)CC1] | 1-(5-((9-(3,3-Dimethylbutyl)- 2,9-diazaspiro[5.5]undecan-2- yl)sulfonyl)pyridin-2- yl)piperidin-2-one | 2.12 | 477.3 | C |
| 123 | 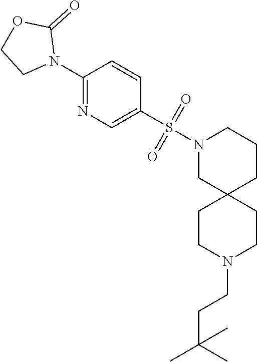 [SMILES: CC(C)(C)CCN1CCC2(CCCN(S(=O)(=O)c3ccc(N4CCOC4=O)nc3)C2)CC1] | 3-(5-((9-(3,3-Dimethylbutyl)- 2,9-diazaspiro[5.5]undecan-2- yl)sulfonyl)pyridin-2- yl)oxazolidin-2-one | 2.05 | 465.2 | B |
| 124 | 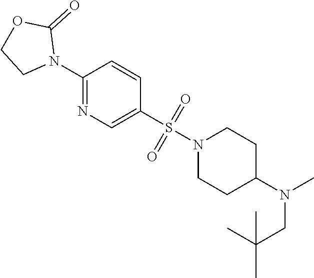 [SMILES: CN(CC(C)(C)C)C1CCN(S(=O)(=O)c2ccc(N3CCOC3=O)nc2)CC1] | 3-(5-((4- (Methyl(neopentyl)amino)pi- peridin-1-yl)sulfonyl)pyridin-2- yl)oxazolidin-2-one | 2.38 | 411.2 | B |
| 125 | 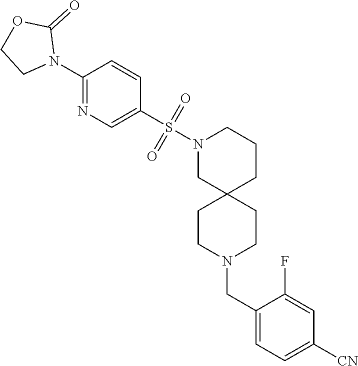 [SMILES: N#Cc1ccc(CN2CCC3(CCCN(S(=O)(=O)c4ccc(N5CCOC5=O)nc4)C3)CC2)c(F)c1] | 3-Fluoro-4-((2-((6-(2- oxooxazolidin-3-yl)pyridin- 3-yl)sulfonyl)-2,9- diazaspiro[5.5]undecan-9- yl)methyl)benzonitrile | 2.17 | 513.9 | C |
| 126 | 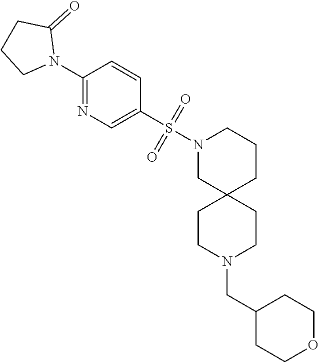 [SMILES: O=C1CCCN1c1ccc(S(=O)(=O)N2CCCC3(CCN(CC4CCOCC4)CC3)C2)cn1] | 1-(5-((9-((Tetrahydro-2H-pyran- 4-yl)methyl)-2,9- diazaspiro[5.5]undecan-2- yl)sulfonyl)pyridin-2- yl)pyrrolidin-2-one | 1.81 | 477.2 | B |
| 127 | 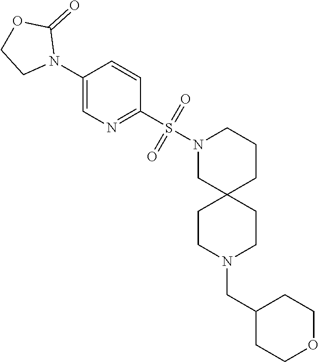 [SMILES: O=C1OCCN1c1ccc(S(=O)(=O)N2CCCC3(CCN(CC4CCOCC4)CC3)C2)nc1] | 3-(6-((9-((Tetrahydro-2H-pyran- 4-yl)methyl)-2,9- diazaspiro[5.5]undecan-2- yl)sulfonyl)pyridin-3- yl)oxazolidin-2-one | 1.54 | 479.0 | B |

### Example 128. 3-(5-((9-((Tetrahydro-2H-pyran-4-yl)methyl)-3,9-diazaspiro[5.5]undecan-3-yl)sulfonyl)pyridin-2-yl)oxazolidin-2-one

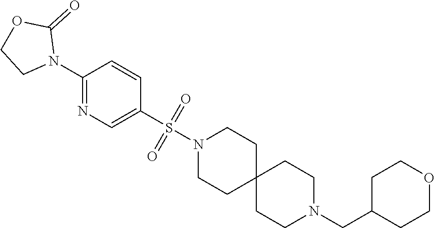 [SMILES: O=C1OCCN1c1ccc(S(=O)(=O)N2CCC3(CCN(CC4CCOCC4)CC3)CC2)cn1]

### Step 1. 3-((6-Chloropyridin-3-yl)sulfonyl)-3,9-diazaspiro[5.5]undecane hydrochloride

Starting from 6-chloropyridine-3-sulfonyl chloride and tert-butyl 3,9-diazaspiro[5.5]undecane-3-carboxylate and following the experimental procedure described in steps 1 and 2 of example 94, the title compound was obtained.

HPLC Rt (Method B): 1.28 min; ESI+-MS m/z: 330.0 (M+H)⁺.

### Step 2. 3-((6-Chloropyridin-3-yl)sulfonyl)-9-((tetrahydro-2H-pyran-4-yl)methyl)-3,9-diazaspiro[5.5]undecane

Starting from the compound obtained in step 1 (100 mg, 0.27 mmol) and following a similar procedure to that described in step 1 of intermediate 20, the title compound was obtained (129 mg, 93% yield).

HPLC Rt (Method B): 1.83 min; ESI+-MS m/z: 428.0 (M+H)⁺.

### Step 3. Title Compound

Starting from the compound obtained in step 2 (116 mg, 0.23 mmol) and following a similar procedure to that described in example 120, the title compound was obtained (68 mg, 62% yield).

HPLC Rt (Method B): 1.68 min; ESI+-MS m/z: 479.2 (M+H)⁺.

This method was used for the preparation of Example 129 using suitable starting materials:

| | | | Rt | MS | HPLC |
|---|---|---|---|---|---|
| EX | STRUCTURE | CHEMICAL NAME | (min) | (M + H) | Method |
| 129 | 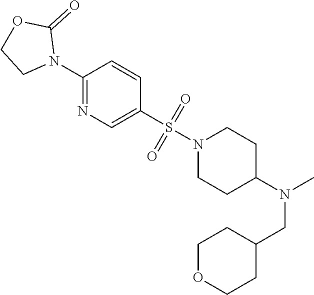 [SMILES: CN(CC1CCOCC1)C1CCN(S(=O)(=O)c2ccc(N3CCOC3=O)nc2)CC1] | 3-(5-((4-(Methyl((tetrahydro- 2H-pyran-4- yl)methyl)amino)piperidin-1- yl)sulfonyl)pyridin-2- yl)oxazolidin-2-one | 1.72 | 439.0 | B |

### Example 130. 9-Benzyl-2-((6-(4,4-difluoropiperidin-1-yl)pyridin-3-yl)sulfonyl)-2,9-diazaspiro[5.5]undecane

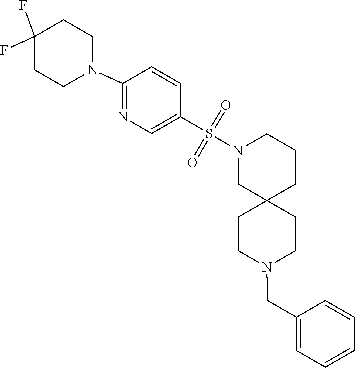 [SMILES: O=S(=O)(c1ccc(N2CCC(F)(F)CC2)nc1)N1CCCC2(CCN(Cc3ccccc3)CC2)C1]

### Step 1. 9-Benzyl-2-((6-chloropyridin-3-yl)sulfonyl)-2,9-diazaspiro[5.5]undecane

Benzyl bromide (0.1 mL, 0.9 mmol) and TEA (0.57 mL, 0.37 mmol) were added to a solution of the product obtained in step 2 of example 94 (300 mg, 0.82 mmol) in DCM (15 mL) and the reaction mixture was stirred at r.t. for 16 h. After this time, benzyl bromide (0.1 mL, 0.9 mmol) was added and the solution was stirred for further 48 h. An aq. sat. NaHCO₃ sol. and DCM were added, the layers were separated and the aqueous phase was extracted with DCM. The combined organic extracts were washed with brine, dried over MgSO₄, filtered and concentrated to dryness. The residue was purified by flash chromatography, silica gel, DCM:MeOH, to obtain the title compound (208 mg, 60% yield).

HPLC Rt (Method B): 2.45 min; ESI+-MS m/z: 420.0 (M+H)⁺.

### Step 2. Title Compound

Starting from the compound obtained in step 1 (60 mg, 0.14 mmol) and following a similar procedure to that described in step 2 of example 20, the title compound was obtained (34 mg, 47% yield).

HPLC Rt (Method B): 2.62 min; ESI+-MS m/z: 505.2 (M+H)⁺.

### Example 131. 4-(5-((9-(3,3-Dimethylbutyl)-3,9-diazaspiro[5.5]undecan-3-yl)sulfonyl)pyridin-2-yl)morpholin-3-one

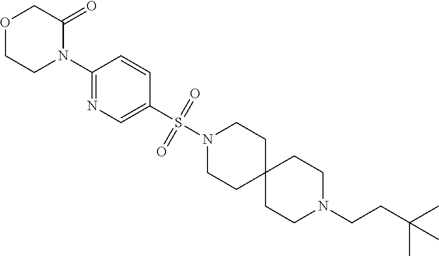 [SMILES: CC(C)(C)CCN1CCC2(CC1)CCN(S(=O)(=O)c1ccc(N3CCOCC3=O)nc1)CC2]

### Step 1. 3-((6-Chloropyridin-3-yl)sulfonyl)-9-(3,3-dimethylbutyl)-3,9-diazaspiro[5.5]undecane

Starting from intermediate 4 (dihydrochloride salt, 400 mg, 1.3 mmol) and 6-chloropyridine-3-sulfonyl chloride (250 mg, 1.2 mmol) and following a similar procedure to that described in step 2 of example 20, the title compound was obtained (470 mg, 96% yield).

HPLC Rt (Method B): 2.14 min; ESI+-MS m/z: 414.2 (M+H)⁺.

### Step 2. Title Compound

Starting from the compound obtained in step 1 (97 mg, 0.23 mmol) and following a similar procedure to that described in example 120, the title compound was obtained (63 mg, 56% yield).

HPLC Rt (Method C): 1.90 min; ESI+-MS m/z: 479.3 (M+H)⁺.

This method was used for the preparation of Examples 132-154 using suitable starting materials:

| | | | Rt | MS | HPLC |
|---|---|---|---|---|---|
| EX | STRUCTURE | CHEMICAL NAME | (min) | (M + H) | Method |
| 132 | 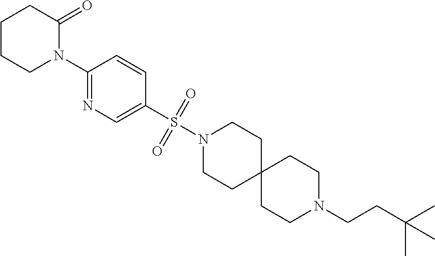 [SMILES: CC(C)(C)CCN1CCC2(CC1)CCN(S(=O)(=O)c1ccc(N3CCCCC3=O)nc1)CC2] | 1-(5-((9-(3,3- Dimethylbutyl)-3,9- diazaspiro[5.5]undecan- 3-yl)sulfonyl)pyridin-2- yl)piperidin-2-one | 2.01 | 477.3 | C |
| 133 | 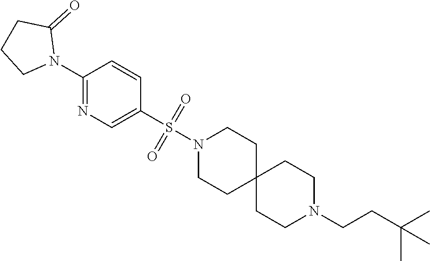 [SMILES: CC(C)(C)CCN1CCC2(CC1)CCN(S(=O)(=O)c1ccc(N3CCCC3=O)nc1)CC2] | 1-(5-((9-(3,3- Dimethylbutyl)-3,9- diazaspiro[5.5]undecan- 3-yl)sulfonyl)pyridin-2- yl)pyrrolidin-2-one | 2.04 | 463.3 | C |
| 134 | 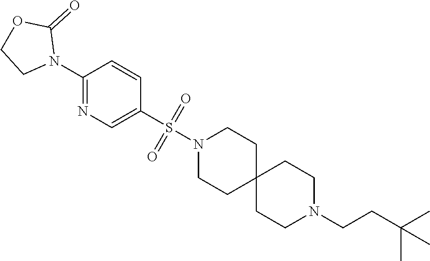 [SMILES: CC(C)(C)CCN1CCC2(CC1)CCN(S(=O)(=O)c1ccc(N3CCOC3=O)nc1)CC2] | 3-(5-((9-(3,3- Dimethylbutyl)-3,9- diazaspiro[5.5]undecan- 3-yl)sulfonyl)pyridin-2- yl)oxazolidin-2-one | 1.96 | 465.0 | B |
| 135 | 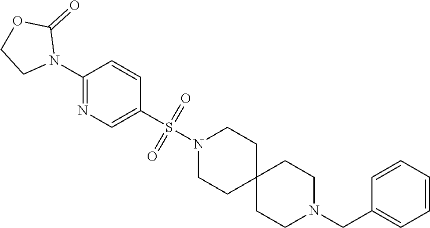 [SMILES: O=C1OCCN1c1ccc(S(=O)(=O)N2CCC3(CCN(Cc4ccccc4)CC3)CC2)cn1] | 3-(5-((9-Benzyl-3,9- diazaspiro[5.5]undecan- 3-yl)sulfonyl)pyridin-2- yl)oxazolidin-2-one | 2.19 | 471.0 | B |
| 136 | 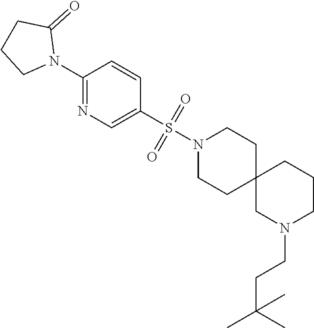 [SMILES: CC(C)(C)CCN1CCCC2(CCN(S(=O)(=O)c3ccc(N4CCCC4=O)nc3)CC2)C1] | 1-(5-((2-(3,3- Dimethylbutyl)-2,9- diazaspiro[5.5]undecan- 9-yl)sulfonyl)pyridin-2- yl)pyrrolidin-2-one | 3.46 | 463.2 | B |
| 137 | 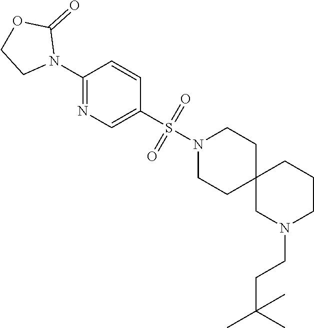 [SMILES: CC(C)(C)CCN1CCCC2(CCN(S(=O)(=O)c3ccc(N4CCOC4=O)nc3)CC2)C1] | 3-(5-((2-(3,3- Dimethylbutyl)-2,9- diazaspiro[5.5]undecan- 9-yl)sulfonyl)pyridin-2- yl)oxazolidin-2-one | 2.49 | 465.2 | B |
| 138 | 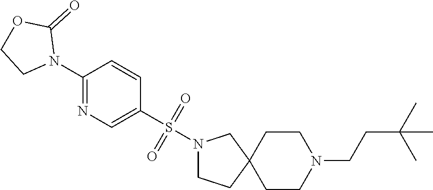 [SMILES: CC(C)(C)CCN1CCC2(CC1)CCN(S(=O)(=O)c1ccc(N3CCOC3=O)nc1)C2] | 3-(5-((8-(3,3- Dimethylbutyl)-2,8- diazaspiro[4.5]decan-2- yl)sulfonyl)pyridin-2- yl)oxazolidin-2-one | 2.05 | 451.2 | B |
| 139 | 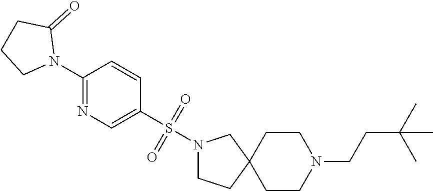 [SMILES: CC(C)(C)CCN1CCC2(CC1)CCN(S(=O)(=O)c1ccc(N3CCCC3=O)nc1)C2] | 1-(5-((8-(3,3- Dimethylbutyl)-2,8- diazaspiro[4.5]decan-2- yl)sulfonyl)pyridin-2- yl)pyrrolidin-2-one | 2.12 | 449.2 | B |
| 140 | 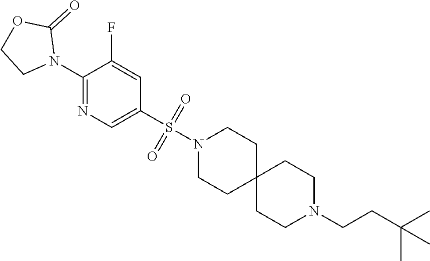 [SMILES: CC(C)(C)CCN1CCC2(CC1)CCN(S(=O)(=O)c1cnc(N3CCOC3=O)c(F)c1)CC2] | 3-(5-((9-(3,3- Dimethylbutyl)-3,9- diazaspiro[5.5]undecan- 3-yl)sulfonyl)-3- fluoropyridin-2- yl)oxazolidin-2-one | 2.00 | 483.2 | B |
| 141 | 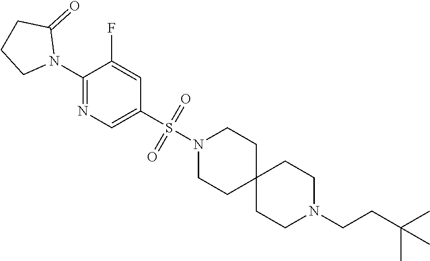 [SMILES: CC(C)(C)CCN1CCC2(CC1)CCN(S(=O)(=O)c1cnc(N3CCCC3=O)c(F)c1)CC2] | 1-(5-((9-(3,3- Dimethylbutyl)-3,9- diazaspiro[5.5]undecan- 3-yl)sulfonyl)-3- fluoropyridin-2- yl)pyrrolidin-2-one | 1.99 | 481.2 | B |
| 142 | 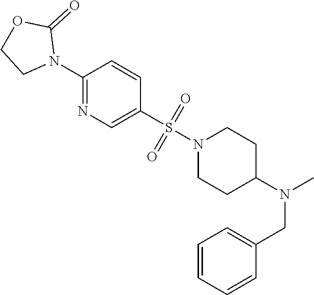 [SMILES: CN(Cc1ccccc1)C1CCN(S(=O)(=O)c2ccc(N3CCOC3=O)nc2)CC1] | 3-(5-((4- (Benzyl(methyl)ami- no)piperidin-1- yl)sulfonyl)pyridin-2- yl)oxazolidin-2-one | 2.17 | 431.0 | B |
| 143 | 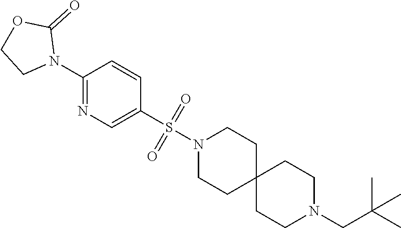 [SMILES: CC(C)(C)CN1CCC2(CC1)CCN(S(=O)(=O)c1ccc(N3CCOC3=O)nc1)CC2] | 3-(5-((9-Neopentyl-3,9- diazaspiro[5.5]undecan- 3-yl)sulfonyl)pyridin-2- yl)oxazolidin-2-one | 2.50 | 451.0 | C |
| 144 | 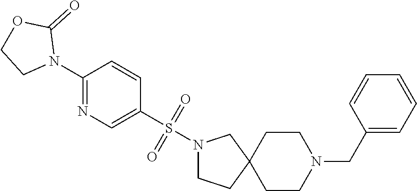 [SMILES: O=C1OCCN1c1ccc(S(=O)(=O)N2CCC3(CCN(Cc4ccccc4)CC3)C2)cn1] | 3-(5-((8-Benzyl-2,8- diazaspiro[4.5]decan-2- yl)sulfonyl)pyridin-2- yl)oxazolidin-2-one | 2.08 | 457.0 | B |
| 145 | 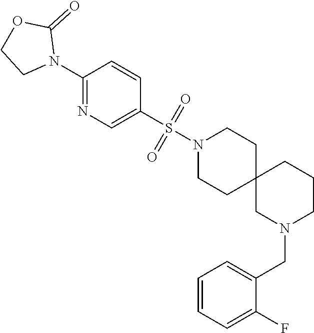 [SMILES: O=C1OCCN1c1ccc(S(=O)(=O)N2CCC3(CCCN(Cc4ccccc4F)C3)CC2)cn1] | 3-(5-((2-(2- Fluorobenzyl)-2,9- diazaspiro[5.5]undecan- 9-yl)sulfonyl)pyridin-2- yl)oxazolidin-2-one | 2.53 | 489.0 | B |
| 146 | 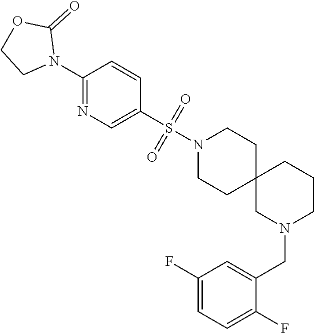 [SMILES: O=C1OCCN1c1ccc(S(=O)(=O)N2CCC3(CCCN(Cc4cc(F)ccc4F)C3)CC2)cn1] | 3-(5-((2-(2,5- Difluorobenzyl)-2,9- diazaspiro[5.5]undecan- 9-yl)sulfonyl)pyridin-2- yl)oxazolidin-2-one | 2.55 | 507.0 | B |
| 147 | 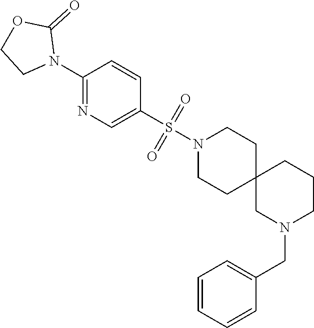 [SMILES: O=C1OCCN1c1ccc(S(=O)(=O)N2CCC3(CCCN(Cc4ccccc4)C3)CC2)cn1] | 3-(5-((2-Benzyl-2,9- diazaspiro[5.5]undecan- 9-yl)sulfonyl)pyridin-2- yl)oxazolidin-2-one | 2.52 | 471.0 | B |
| 148 | 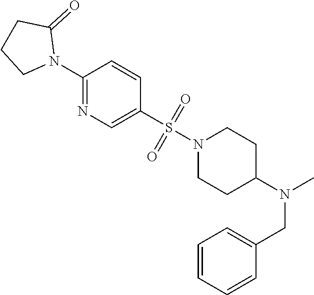 [SMILES: CN(Cc1ccccc1)C1CCN(S(=O)(=O)c2ccc(N3CCCC3=O)nc2)CC1] | 1-(5-((4- (Benzyl(methyl)ami- no)piperidin-1- yl)sulfonyl)pyridin-2- yl)pyrrolidin-2-one | 2.20 | 429.0 | B |
| 149 | 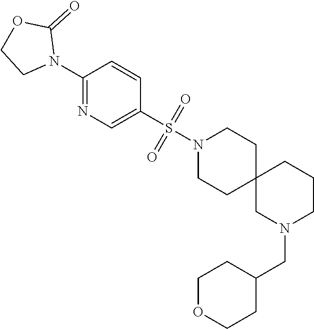 [SMILES: O=C1OCCN1c1ccc(S(=O)(=O)N2CCC3(CCCN(CC4CCOCC4)C3)CC2)cn1] | 3-(5-((2-((Tetrahydro- 2H-pyran-4-yl)methyl)- 2,9- diazaspiro[5.5]undecan- 9-yl)sulfonyl)pyridin-2- yl)oxazolidin-2-one | 2.20 | 479.0 | C |
| 150 | 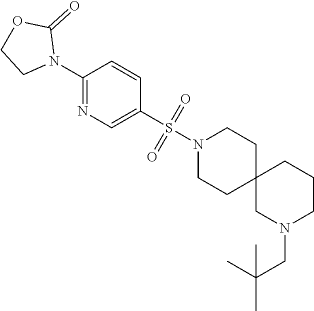 [SMILES: CC(C)(C)CN1CCCC2(CCN(S(=O)(=O)c3ccc(N4CCOC4=O)nc3)CC2)C1] | 3-(5-((2-Neopentyl-2,9- diazaspiro[5.5]undecan- 9-yl)sulfonyl)pyridin-2- yl)oxazolidin-2-one | 2.84 | 451.0 | C |
| 151 | 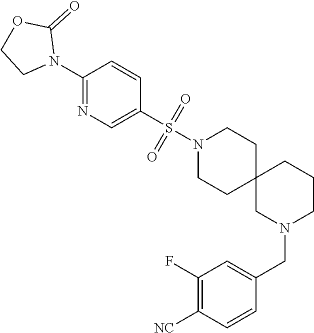 [SMILES: N#Cc1ccc(CN2CCCC3(CCN(S(=O)(=O)c4ccc(N5CCOC5=O)nc4)CC3)C2)cc1F] | 2-Fluoro-4-((9-((6-(2- oxooxazolidin-3- yl)pyridin-3- yl)sulfonyl)-2,9- diazaspiro[5.5]undecan- 2-yl)methyl)benzonitrile | 2.41 | 514.0 | B |
| 152 | 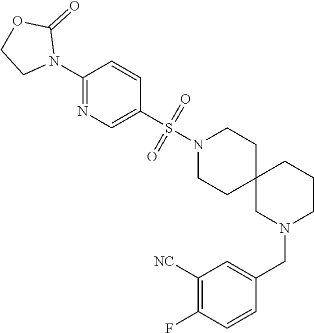 [SMILES: N#Cc1cc(CN2CCCC3(CCN(S(=O)(=O)c4ccc(N5CCOC5=O)nc4)CC3)C2)ccc1F] | 2-Fluoro-5-((9-((6-(2- oxooxazolidin-3- yl)pyridin-3- yl)sulfonyl)-2,9- diazaspiro[5.5]undecan- 2-yl)methyl)benzonitrile | 2.40 | 514.0 | B |
| 153 | 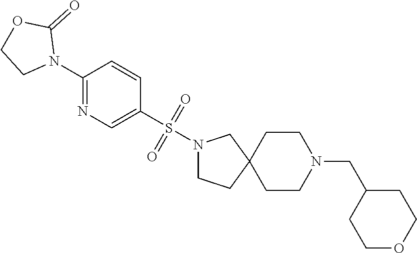 [SMILES: O=C1OCCN1c1ccc(S(=O)(=O)N2CCC3(CCN(CC4CCOCC4)CC3)C2)cn1] | 3-(5-((8-((Tetrahydro- 2H-pyran-4-yl)methyl)- 2,8-diazaspiro[4.5]decan- 2-yl)sulfonyl)pyridin-2- yl)oxazolidin-2-one | 1.67 | 465.2 | B |
| 154 | 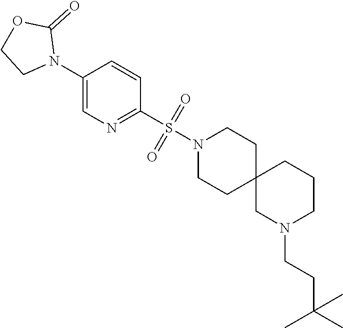 [SMILES: CC(C)(C)CCN1CCCC2(CCN(S(=O)(=O)c3ccc(N4CCOC4=O)cn3)CC2)C1] | 3-(6-((2-(3,3- Dimethylbutyl)-2,9- diazaspiro[5.5]undecan- 9-yl)sulfonyl)pyridin-3- yl)oxazolidin-2-one | 2.25 | 465.2 | B |

### Example 155. 3-(5-((9-Benzyl-2,9-diazaspiro[5.5]undecan-2-yl)sulfonyl)pyridin-2-yl)oxazolidin-2-one

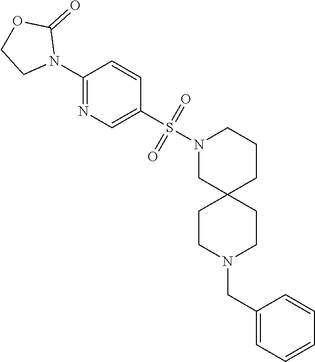 [SMILES: O=C1OCCN1c1ccc(S(=O)(=O)N2CCCC3(CCN(Cc4ccccc4)CC3)C2)cn1]

Starting from the compound obtained in step 1 of example 130 (76 mg, 0.18 mmol) and oxazolidin-2-one (19 mg, 0.22 mmol), following a similar procedure to that described in example 120, the title compound was obtained (29 mg, 33% yield).

HPLC Rt (Method B): 2.25 min; ESI+-MS m/z: 471.0 (M+H)⁺.

This method was used for the preparation of Examples 156 and 157 using suitable starting materials:

| | | | Rt | MS | HPLC |
|---|---|---|---|---|---|
| EX | STRUCTURE | CHEMICAL NAME | (min) | (M + H) | Method |
| 156 | 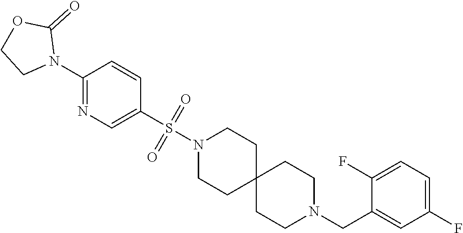 [SMILES: O=C1OCCN1c1ccc(S(=O)(=O)N2CCC3(CCN(Cc4cc(F)ccc4F)CC3)CC2)cn1] | 3-(5-((9-(2,5-Difluorobenzyl)- 3,9-diazaspiro[5.5]undecan- yl)sulfonyl)pyridin-2- yl)oxazolidin-3-2-one | 2.30 | 507.0 | B |
| 157 | 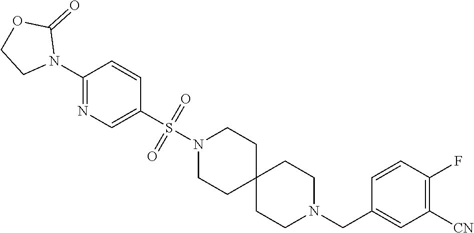 [SMILES: N#Cc1cc(CN2CCC3(CC2)CCN(S(=O)(=O)c2ccc(N4CCOC4=O)nc2)CC3)ccc1F] | 2-Fluoro-5-((9-((6-(2- oxooxazolidin-3-yl)pyridin- 3-yl)sulfonyl)-3,9- diazaspiro[5.5]undecan-3- yl)methyl)benzonitrile | 2.16 | 514.0 | B |

### Example 158. 3-(5-((9-((Tetrahydro-2H-pyran-4-yl)methyl)-2,9-diazaspiro[5.5]undecan-2-yl)sulfonyl)pyridin-2-yl)oxazolidin-2-one

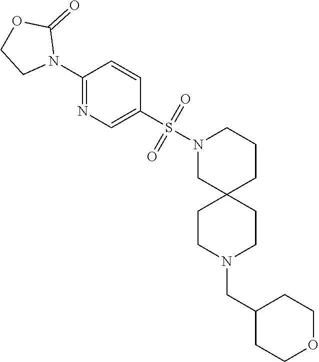 [SMILES: O=C1OCCN1c1ccc(S(=O)(=O)N2CCCC3(CCN(CC4CCOCC4)CC3)C2)cn1]

### Step 1. tert-Butyl 2-((6-(2-oxooxazolidin-3-yl)pyridin-3-yl)sulfonyl)-2,9-diazaspiro[5.5]undecane-9-carboxylate

Starting from the compound obtained in step 1 of Example 94 (796 mg, 1.8 mmol) and following a similar procedure to that described in example 120, the title compound was obtained (560 mg, 63% yield).

HPLC Rt (Method C): 2.20 min; ESI+-MS m/z: 481.1 (M+H)⁺.

### Step 2. 3-(5-((2,9-Diazaspiro[5.5]undecan-2-yl)sulfonyl)pyridin-2-yl)oxazolidin-2-one hydrochloride

Starting from the compound obtained in step 1 (365 mg, 0.76 mmol) and following a similar procedure to that described in step 2 of example 94, the title compound was obtained (350 mg, Quant.).

HPLC Rt (Method B): 1.22 min; ESI+-MS m/z: 381.0 (M+H)⁺.

### Step 3. Title Compound

Starting from the compound obtained in step 2 (91 mg, 0.22 mmol) and following a similar procedure to that described in step 1 of intermediate 20, the title compound was obtained (64 mg, 66% yield).

HPLC Rt (Method B): 1.74 min; ESI+-MS m/z: 479.0 (M+H)⁺.

### Example 159. 3-(5-((9-(2-Fluorobenzyl)-2,9-diazaspiro[5.5]undecan-2-yl)sulfonyl)pyridin-2-yl)oxazolidin-2-one

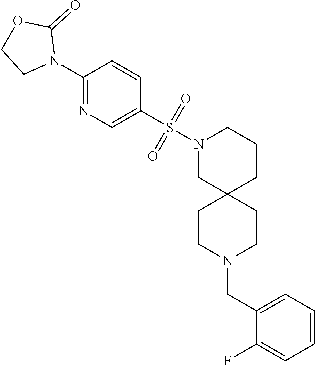 [SMILES: O=C1OCCN1c1ccc(S(=O)(=O)N2CCCC3(CCN(Cc4ccccc4F)CC3)C2)cn1]

Starting from the compound obtained in step 2 of example 158 (100 mg, 0.22 mmol) and following a similar procedure to that described in step 3 of example 61, the title compound was obtained (16 mg, 15% yield).

HPLC Rt (Method B): 2.24 min; ESI+-MS m/z: 489.0 (M+H)⁺.

This method was used for the preparation of Examples 160-166 using suitable starting materials:

| | | | Rt | MS | HPLC |
|---|---|---|---|---|---|
| EX | STRUCTURE | CHEMICAL NAME | (min) | (M + H) | Method |
| 160 | 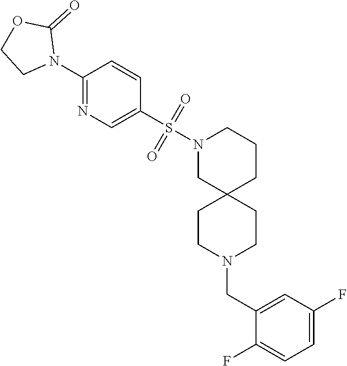 [SMILES: O=C1OCCN1c1ccc(S(=O)(=O)N2CCCC3(CCN(Cc4cc(F)ccc4F)CC3)C2)cn1] | 3-(5-((9-(2,5- Difluorobenzyl)-2,9- diazaspiro[5.5]undecan-2- yl)sulfonyl)pyridin-2- yl)oxazolidin-2-one | 2.33 | 507.0 | B |
| 161 | 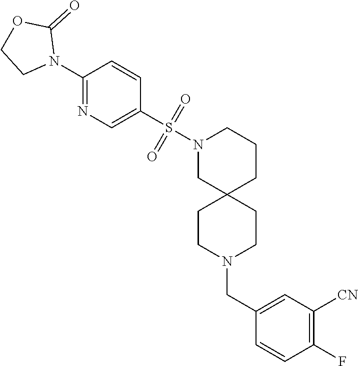 [SMILES: N#Cc1cc(CN2CCC3(CCCN(S(=O)(=O)c4ccc(N5CCOC5=O)nc4)C3)CC2)ccc1F] | 2-Fluoro-5-((2-((6-(2- oxooxazolidin-3- yl)pyridin-3- yl)sulfonyl)-2,9- diazaspiro[5.5]undecan- 9-yl)methyl)benzonitrile | 2.20 | 514.0 | B |
| 162 | 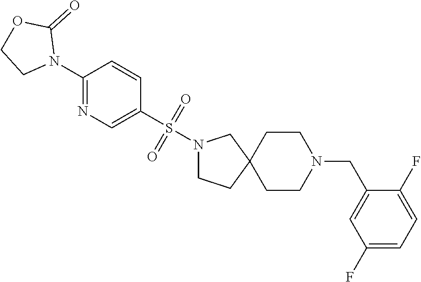 [SMILES: O=C1OCCN1c1ccc(S(=O)(=O)N2CCC3(CCN(Cc4cc(F)ccc4F)CC3)C2)cn1] | 3-(5-((8-(2,5- Difluorobenzyl)-2,8- diazaspiro[4.5]decan- 2-yl)sulfonyl)pyridin- 2-yl)oxazolidin-2-one | 2.18 | 493.0 | B |
| 163 | 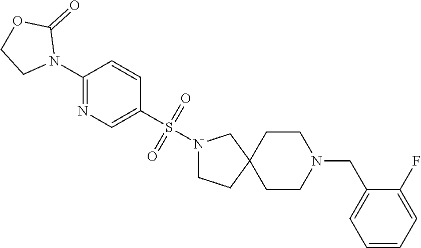 [SMILES: O=C1OCCN1c1ccc(S(=O)(=O)N2CCC3(CCN(Cc4ccccc4F)CC3)C2)cn1] | 3-(5-((8-(2-Fluorobenzyl)- 2,8-diazaspiro[4.5]decan- 2-yl)sulfonyl)pyridin-2- yl)oxazolidin-2-one | 2.11 | 475.0 | B |
| 164 | 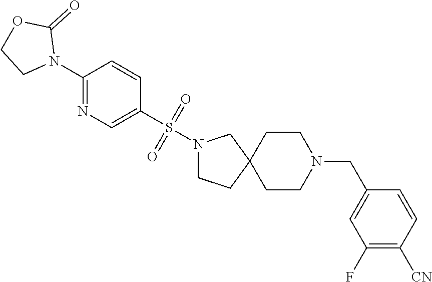 [SMILES: N#Cc1ccc(CN2CCC3(CC2)CCN(S(=O)(=O)c2ccc(N4CCOC4=O)nc2)C3)cc1F] | 2-Fluoro-4-((2-((6-(2- oxooxazolidin-3- yl)pyridin-3-yl)sulfonyl)- 2,8-diazaspiro[4.5]decan- 8-yl)methyl)benzonitrile | 2.11 | 500.0 | B |
| 165 | 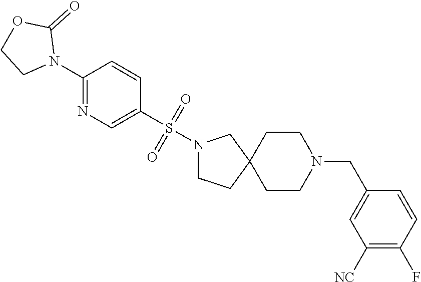 [SMILES: N#Cc1cc(CN2CCC3(CC2)CCN(S(=O)(=O)c2ccc(N4CCOC4=O)nc2)C3)ccc1F] | 2-Fluoro-5-((2-((6-(2- oxooxazolidin-3- yl)pyridin-3-yl)sulfonyl)- 2,8-diazaspiro[4.5]decan- 8-yl)methyl)benzonitrile | 2.07 | 500.0 | B |
| 166 | 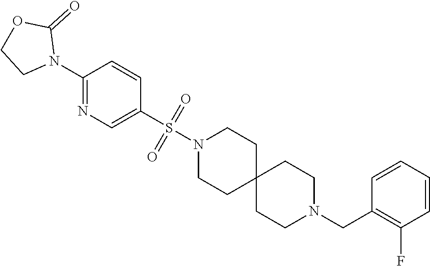 [SMILES: O=C1OCCN1c1ccc(S(=O)(=O)N2CCC3(CCN(Cc4ccccc4F)CC3)CC2)cn1] | 3-(5-((9-(2-Fluorobenzyl)- 3,9-diazaspiro[5.5]undecan- 3-yl)sulfonyl)pyridin-2- yl)oxazolidin-2-one | 2.20 | 489.0 | B |

### Example 167. N-((1 r,4r)-4-(Benzyl(methyl)amino)cyclohexyl)-6-(2-oxooxazolidin-3-yl)pyridine-3-sulfonamide

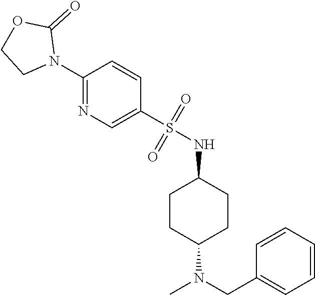 [SMILES: CN(Cc1ccccc1)[C@H]1CC[C@H](NS(=O)(=O)c2ccc(N3CCOC3=O)nc2)CC1]

### Step 1. tert-Butyl ((1 r,4r)-4-((6-chloropyridine)-3-sulfonamido)cyclohexyl)carbamate

Starting from 6-chloropyridine-3-sulfonyl chloride (1.88 g, 8.86 mmol) and tert-butyl ((1r,4r)-4-aminocyclohexyl)carbamate (1.9 g, 8.86 mmol), following the experimental procedure described in example 13, the title compound was obtained (1.61 g, 47% yield).

HPLC Rt (Method B): 2.04 min; ESI+-MS m/z: 388.0 (M−H)⁻.

### Step 2. N-((1 r,4r)-4-Aminocyclohexyl)-6-chloropyridine-3-sulfonamide trifluoroacetate

Starting from the compound obtained in step 1 (454 mg, 1.16 mmol) and following a similar procedure to that described in step 2a of intermediate 4, the title compound was obtained (574 mg, Quant.).

HPLC Rt (Method B): 1.14 min; ESI+-MS m/z: 290.0 (M+H)⁺.

### Step 3. N-((1r,4r)-4-(Benzylamino)cyclohexyl)-6-chloropyridine-3-sulfonamide

Starting from the compound obtained in step 2 (216 mg, 0.74 mmol) (with TEA, 0.52 mL, 3.7 mmol) and following a similar procedure to that described in step 1 of intermediate 1, the title compound was obtained (200 mg, 71% yield).

HPLC Rt (Method B): 1.78 min; ESI+-MS m/z: 380.0 (M+H)⁺.

### Step 4. N-((1 r,4r)-4-(Benzyl(methyl)amino)cyclohexyl)-6-chloropyridine-3-sulfonamide

Starting from the compound obtained in step 4 (200 mg, 0.53 mmol) and following a similar procedure to that described in example 56, the title compound was obtained (202 mg, 93% yield).

HPLC Rt (Method B): 2.23 min; ESI+-MS m/z: 394.0 (M+H)⁺.

### Step 5. Title Compound

Starting from the compound obtained in step 4 (100 mg, 0.25 mmol) and following a similar procedure to that described in example 120, the title compound was obtained (84 mg, 74% yield).

HPLC Rt (Method B): 1.92 min; ESI+-MS m/z: 445.0 (M+H)⁺.

### Example 168. 1-((6-(4,4-Difluoropiperidin-1-yl)pyridin-3-yl)sulfonyl)-N-(3,3-dimethylbutyl)-N-methylpiperidin-4-amine

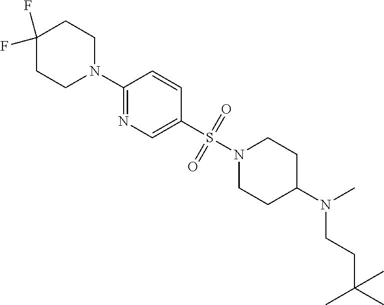 [SMILES: CN(CCC(C)(C)C)C1CCN(S(=O)(=O)c2ccc(N3CCC(F)(F)CC3)nc2)CC1]

### Step 1. tert-Butyl (1-((6-chloropyridin-3-yl)sulfonyl)piperidin-4-yl)(methyl)carbamate

Starting from 6-chloropyridine-3-sulfonyl chloride (250 mg, 1.18 mmol) and tert-butyl methyl(piperidin-4-yl)carbamate (278 mg, 1.3 mmol) and following the experimental procedure described in example 13, the title compound was obtained (442 mg, 96% yield).

HPLC Rt (Method B): 2.23 min; ESI+-MS m/z: 390.0 (M+H)⁺.

### Step 2. tert-Butyl (1-((6-(4,4-difluoropiperidin-1-yl)pyridin-3-yl)sulfonyl)piperidin-4-yl)(methyl)carbamate

Starting from the compound obtained in step 1 (360 mg, 0.92 mmol) following a similar procedure to that described in step 2 of example 20, the title compound was obtained (422 mg, 96% yield).

HPLC Rt (Method B): 2.38 min; ESI+-MS m/z: 475.0 (M+H)⁺.

### Step 3. 1-((6-(4,4-Difluoropiperidin-1-yl)pyridin-3-yl)sulfonyl)-N-methylpiperidin-4-amine hydrochloride

Starting from the compound obtained in step 2 (442 mg, 0.93 mmol) and following a similar procedure to that described in step 2 of example 94, the title compound was obtained (420 mg, Quant.).

HPLC Rt (Method B): 1.52 min; ESI+-MS m/z: 375.0 (M+H)⁺.

### Step 4. Title Compound

Starting from the compound obtained in step 3 (80 mg, 0.19 mmol) and following a similar procedure to that described in step 3 of example 35, the title compound was obtained (61 mg, 68% yield).

HPLC Rt (Method B): 2.41 min; ESI+-MS m/z: 459.2 (M+H)⁺.

This method was used for the preparation of Examples 169 and 170 using suitable starting materials:

| | | | Rt | MS | HPLC |
|---|---|---|---|---|---|
| EX | STRUCTURE | CHEMICAL NAME | (min) | (M + H) | Method |
| 169 | 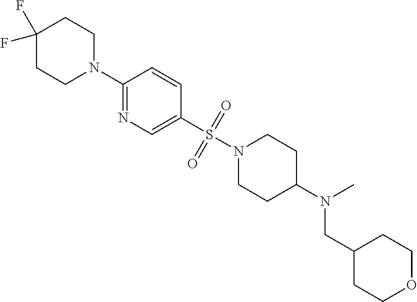 [SMILES: CN(CC1CCOCC1)C1CCN(S(=O)(=O)c2ccc(N3CCC(F)(F)CC3)nc2)CC1] | 1-((6-(4,4-Difluoropiperidin- 1-yl)pyridin-3-yl)sulfonyl)- N-methyl-N-((tetrahydro-2H- pyran-4-yl)methyl)piperidin- 4-amine | 2.12 | 473.2 | B |
| 170 | 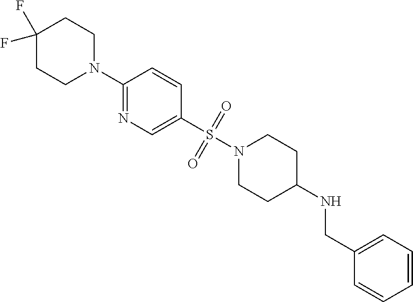 [SMILES: O=S(=O)(c1ccc(N2CCC(F)(F)CC2)nc1)N1CCC(NCc2ccccc2)CC1] | N-Benzyl-1-((6-(4,4- difluoropiperidin-1- yl)pyridin-3-yl)sulfonyl)-N- methylpiperidin-4-amine | 2.19 | 451.0 | B |

### Example 171. 4-(((1-((6-(4,4-Difluoropiperidin-1-yl)pyridin-3-yl)sulfonyl)piperidin-4-yl)(methyl)amino)methyl)-3-fluorobenzonitrile

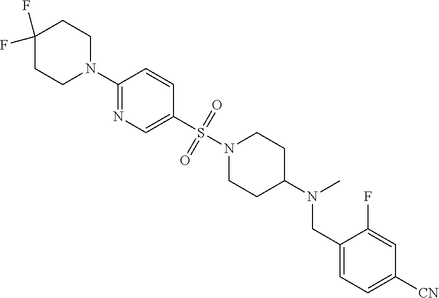 [SMILES: CN(Cc1ccc(C#N)cc1F)C1CCN(S(=O)(=O)c2ccc(N3CCC(F)(F)CC3)nc2)CC1]

Starting from the compound obtained in step 3 of example 168 (83 mg, 0.2 mmol) (with TEA, 0.30 mL, 0.22 mmol) and following a similar procedure to that described in step 1 of intermediate 4, the title compound was obtained (28 mg, 28% yield).

HPLC Rt (Method B): 2.38 min; ESI+-MS m/z: 419.2 (M+H)⁺.

### Example 172. 1-((6-(4,4-Difluoropiperidin-1-yl)pyridin-3-yl)sulfonyl)-N-(2-fluorobenzyl)-N-methylpiperidin-4-amine

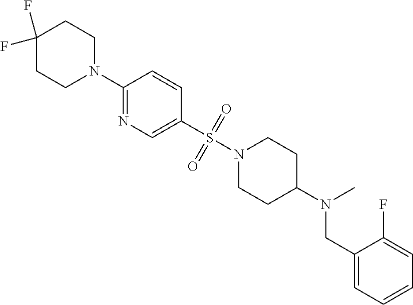 [SMILES: CN(Cc1ccccc1F)C1CCN(S(=O)(=O)c2ccc(N3CCC(F)(F)CC3)nc2)CC1]

Starting from the compound obtained in step 3 of example 168 (80 mg, 0.19 mmol) and following a similar procedure to that described in step 3 of example 61, the title compound was obtained (23 mg, 25% yield).

HPLC Rt (Method B): 2.46 min; ESI+-MS m/z: 483.0 (M+H)⁺.

This method was used for the preparation of Examples 173 and 174 using suitable starting materials:

| | | | Rt | MS | HPLC |
|---|---|---|---|---|---|
| EX | STRUCTURE | CHEMICAL NAME | (min) | (M + H) | Method |
| 173 | 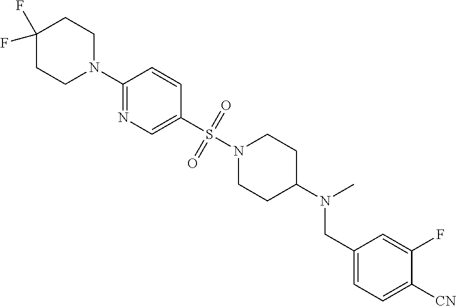 [SMILES: CN(Cc1ccc(C#N)c(F)c1)C1CCN(S(=O)(=O)c2ccc(N3CCC(F)(F)CC3)nc2)CC1] | 4-(((1-((6-(4,4-Difluoropiperidin- 1-yl)pyridin-3- yl)sulfonyl)piperidin-4- yl)(methyl)amino)methyl)-2- fluorobenzonitrile | 2.42 | 508.0 | B |
| 174 | 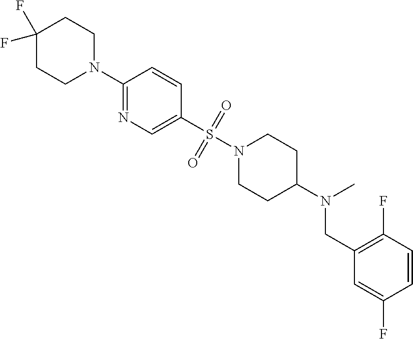 [SMILES: CN(Cc1cc(F)ccc1F)C1CCN(S(=O)(=O)c2ccc(N3CCC(F)(F)CC3)nc2)CC1] | N-(2,5-Difluorobenzyl)-1-((6- (4,4-difluoropiperidin-1- yl)pyridin-3-yl)sulfonyl)-N- methylpiperidin-4-amine | 2.52 | 501.0 | B |

### Example 175. N-((1r,4r)-4-(Benzyl(methyl)amino)cyclohexyl)-6-(4,4-difluoropiperidin-1-yl)pyridine-3-sulfonamide

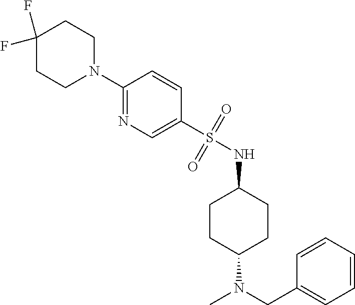 [SMILES: CN(Cc1ccccc1)[C@H]1CC[C@H](NS(=O)(=O)c2ccc(N3CCC(F)(F)CC3)nc2)CC1]

### Step 1. tert-Butyl ((1r,4r)-4-((6-(4,4-difluoropiperidin-1-yl)pyridine)-3-sulfonamido)cyclohexyl)carbamate

Starting from the compound obtained in step 1 of example 167 (300 mg, 0.77 mmol) and following a similar procedure to that described in step 2 of example 20, the title compound was obtained (331 mg, 91% yield).

HPLC Rt (Method B): 2.20 min; ESI+-MS m/z: 475.2 (M+H)⁺.

### Step 2. N-((1 r,4r)-4-Aminocyclohexyl)-6-(4,4-difluoropiperidin-1-yl)pyridine-3-sulfonamide trifluoroacetate

Starting from the compound obtained in step 1 (331 mg, 0.7 mmol) and following a similar procedure to that described in step 2a of intermediate 4, the title compound was obtained (472 mg, Quant.).

HPLC Rt (Method B): 1.50 min; ESI+-MS m/z: 375.0 (M+H)⁺.

### Step 3. N-((1 r,4r)-4-(Benzylamino)cyclohexyl)-6-(4,4-difluoropiperidin-1-yl)pyridine-3-sulfonamide

Starting from the compound obtained in step 2 (420 mg, 0.7 mmol) (with TEA, 0.39 mL, 2.8 mmol) and following a similar procedure to that described in step 1 of intermediate 1, the title compound was obtained (78 mg, 24% yield).

HPLC Rt (Method B): 2.00 min; ESI+-MS m/z: 465.0 (M+H)⁺.

### Step 4. Title Compound

Starting from the compound obtained in step 3 (78 mg, 0.17 mmol) and following a similar procedure to that described in example 56, the title compound was obtained (52 mg, 65% yield).

HPLC Rt (Method B): 2.35 min; ESI+-MS m/z: 479.0 (M+H)⁺.

This method was used for the preparation of Examples 176-183 using suitable starting materials:

| | | | Rt | MS | HPLC |
|---|---|---|---|---|---|
| EX | STRUCTURE | CHEMICAL NAME | (min) | (M + H) | Method |
| 176 | 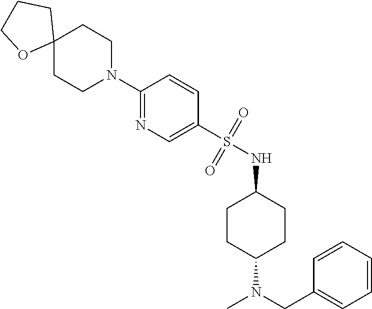 [SMILES: CN(Cc1ccccc1)[C@H]1CC[C@H](NS(=O)(=O)c2ccc(N3CCC4(CCCO4)CC3)nc2)CC1] | N-((1r,4r)-4-(Benzyl(meth- yl)amino)cyclohexyl)- 6-(1-oxa-8-azaspiro[4.5]decan-8- yl)pyridine-3-sulfonamide | 2.24 | 499.0 | C |
| 177 | 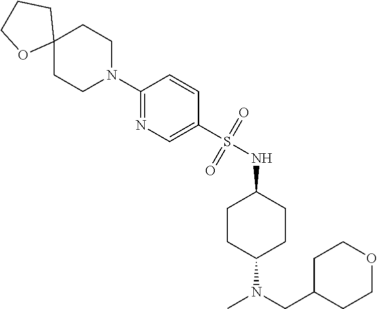 [SMILES: CN(CC1CCOCC1)[C@H]1CC[C@H](NS(=O)(=O)c2ccc(N3CCC4(CCCO4)CC3)nc2)CC1] | N-((1r,4r)-4-(Methyl((tetrahydro- 2H-pyran-4- yl)methyl)amino)cyclohexyl)-6- (1-oxa-8-azaspiro[4.5]decan-8- yl)pyridine-3-sulfonamide | 1.66 | 507.0 | C |
| 178 | 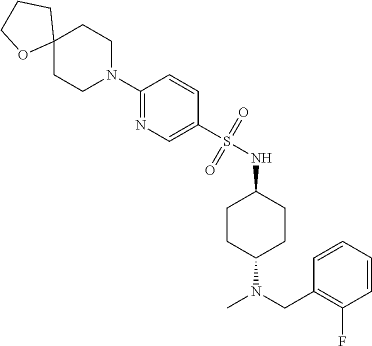 [SMILES: CN(Cc1ccccc1F)[C@H]1CC[C@H](NS(=O)(=O)c2ccc(N3CCC4(CCCO4)CC3)nc2)CC1] | N-((1r,4r)-4-((2-Fluorobenz- yl)(methyl)amino)cyclohexyl)- 6-(1-oxa-8-azaspiro[4.5]decan-8- yl)pyridine-3-sulfonamide | 2.32 | 517.0 | C |
| 179 | 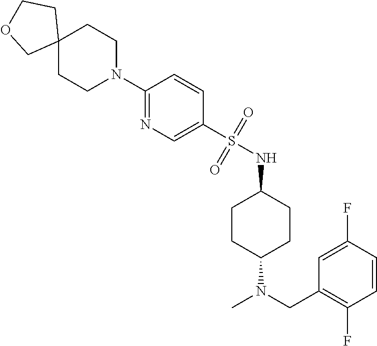 [SMILES: CN(Cc1cc(F)ccc1F)[C@H]1CC[C@H](NS(=O)(=O)c2ccc(N3CCC4(CCOC4)CC3)nc2)CC1] | N-((1r,4r)-4-((2,5-Difluoro- benzyl)(methyl)amino)cyclohexyl)- 6-(2-oxa-8-azaspiro[4.5]decan-8- yl)pyridine-3-sulfonamide | 2.30 | 535.2 | B |
| 180 | 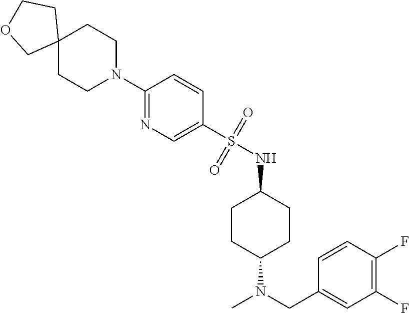 [SMILES: CN(Cc1ccc(F)c(F)c1)[C@H]1CC[C@H](NS(=O)(=O)c2ccc(N3CCC4(CCOC4)CC3)nc2)CC1] | N-((1r,4r)-4-((3,4-Difluoro- benzyl)(methyl)amino)cyclohexyl)- 6-(2-oxa-8-azaspiro[4.5]decan-8- yl)pyridine-3-sulfonamide | 2.32 | 535.2 | B |
| 181 | 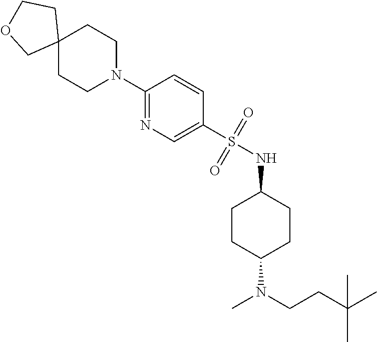 [SMILES: CN(CCC(C)(C)C)[C@H]1CC[C@H](NS(=O)(=O)c2ccc(N3CCC4(CCOC4)CC3)nc2)CC1] | N-((1r,4r)-4-((3,3-Dimethylbut- yl)(methyl)amino)cyclohexyl)-6- (2-oxa-8-azaspiro[4.5]decan-8- yl)pyridine-3-sulfonamide | 1.82 | 493.2 | B |
| 182 | 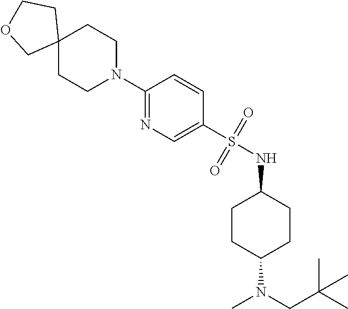 [SMILES: CN(CC(C)(C)C)[C@H]1CC[C@H](NS(=O)(=O)c2ccc(N3CCC4(CCOC4)CC3)nc2)CC1] | N-((1r,4r)-4-(Methyl(neopentyl)a- mino)cyclohexyl)-6-(2-oxa-8- azaspiro[4.5]decan-8-yl)pyridine- 3-sulfonamide | 2.23 | 479.2 | B |
| 183 | 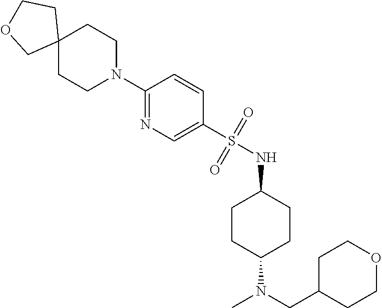 [SMILES: CN(CC1CCOCC1)[C@H]1CC[C@H](NS(=O)(=O)c2ccc(N3CCC4(CCOC4)CC3)nc2)CC1] | N-((1r,4r)-4-(Methyl((tetrahydro- 2H-pyran-4- yl)methyl)amino)cyclohexyl)-6- (2-oxa-8-azaspiro[4.5]decan-8- yl)pyridine-3-sulfonamide | 1.59 | 507.2 | B |

### Examples of Biological Activity

### Pharmacological Study

This invention is aimed at providing a series of compounds which show pharmacological activity towards the σ₁ receptor and/or σ₂ receptor and, especially, compounds which have a binding expressed as Kᵢ responding to the following scales:
Kᵢ(σ₁) is preferably <1000 nM, more preferably <500 nM, even more preferably <100 nM; and
Kᵢ(σ₂) is preferably <1000 nM, more preferably <500 nM, even more preferably <100 nM.

### Human σ₁ Receptor Radioligand Assay

Transfected HEK-293 membranes (7 μg) were incubated with 5 nM of [³H](+)-pentazocine in assay buffer containing Tris-HCl 50 mM at pH 8. NBS (non-specific binding) was measured by adding 10 μM haloperidol. The binding of the test compound was measured at either one concentration (% inhibition at 1 or 10 μM) or five different concentrations to determine affinity values (Ki). Plates were incubated at 37° C. for 120 minutes. After the incubation period, the reaction mix was then transferred to MultiScreen HTS, FC plates (Millipore), filtered and plates were washed 3 times with ice-cold 10 mM Tris-HCL (pH7.4). Filters were dried and counted at approximately 40% efficiency in a MicroBeta scintillation counter (Perkin-Elmer) using EcoScint liquid scintillation cocktail.

### Binding Assay to Human σ2/TMEM97 Receptor

Transfected HEK-293 membranes (15 μg) were incubated with 10 nM [³H]-1,3-Di-o-tolylguanidine (DTG) in assay buffer containing Tris-HCl 50 mM at pH 8.0. NSB (non-specific binding) was measured by adding 10 μM haloperidol. The binding of the test compound was measured at either one concentration (% inhibition at 1 or 10 μM) or five different concentrations to determine affinity values (Ki). Plates were incubated at 25° C. for 120 minutes. After the incubation period, the reaction mix was transferred to MultiScreen HTS, FC plates (Millipore), filtered and washed 3 times with ice-cold 10 mM Tris-HCL (pH 8.0). Filters were dried and counted at approximately 40% efficiency in a MicroBeta scintillation counter (Perkin-Elmer) using EcoScint liquid scintillation cocktail.

### Results:

The following scale has been adopted for representing the binding to σ1-receptor expressed as Kᵢ:
+ Kᵢ (σ₁)>1000 nM or inhibition ranges between 1% and 50%
++ 500 nM<=Kᵢ(σ₁)<=1000 nM
+++ 100 nM<=Kᵢ(σ₁)<=500 nM
++++ Kᵢ(σ₁)<100 nM

The following scale has been adopted for representing the binding to σ2-receptor expressed as Kᵢ:
+ Kᵢ(σ₂)>1000 nM or inhibition ranges between 1% and 50%
++ 500 nM<=Kᵢ(σ₂)<=1000 nM
+++ 100 nM<=Kᵢ(σ₂)<=500 nM
++++ Kᵢ(σ₂)<100 nM

The results of the compounds showing binding for the σ-1 and/or σ-2 receptor are shown in Table 1:

TABLE 1

| | Binding σ-1 | Binding σ-2 |
|---|---|---|
| Example | Kᵢ (nM) | Kᵢ (nM) |
| 1 | ++++ | + |
| 2 | ++++ | + |
| 3 | ++++ | + |
| 4 | ++++ | + |
| 5 | ++++ | + |
| 6 | ++++ | ++ |
| 7 | ++++ | + |
| 8 | ++++ | + |
| 9 | ++ | + |
| 10 | ++++ | + |
| 11 | ++++ | + |
| 12 | + | ++++ |
| 13 | + | +++ |
| 14 | ++++ | + |
| 15 | + | +++ |
| 16 | + | +++ |
| 17 | ++++ | + |
| 18 | + | ++++ |
| 19 | + | ++++ |
| 20 | ++++ | + |
| 21 | ++++ | + |
| 22 | ++++ | + |
| 23 | ++++ | + |
| 24 | ++++ | ++ |
| 25 | ++++ | + |
| 26 | ++ | + |
| 27 | ++++ | + |
| 28 | + | ++++ |
| 29 | +++ | ++++ |
| 30 | ++ | +++ |
| 31 | ++++ | ++++ |
| 32 | +++ | ++++ |
| 33 | + | ++++ |
| 34 | ++ | ++++ |
| 35 | ++++ | + |
| 36 | ++++ | + |
| 37 | ++++ | + |
| 38 | ++++ | + |
| 39 | ++++ | + |
| 40 | +++ | + |
| 41 | ++++ | + |
| 42 | ++ | + |
| 43 | ++++ | + |
| 44 | ++ | + |
| 45 | ++ | +++ |
| 46 | + | +++ |
| 47 | + | +++ |
| 48 | + | ++++ |
| 49 | + | + |
| 50 | ++ | + |
| 51 | + | +++ |
| 52 | + | +++ |
| 53 | + | +++ |
| 54 | +++ | +++ |
| 55 | + | + |
| 56 | + | ++++ |
| 57 | + | ++++ |
| 58 | + | ++++ |
| 59 | + | + |
| 60 | + | +++ |
| 61 | +++ | ++++ |
| 62 | +++ | ++++ |
| 63 | +++ | ++++ |
| 64 | + | +++ |
| 65 | + | +++ |
| 66 | ++++ | ++++ |
| 67 | ++++ | ++++ |
| 68 | +++ | ++++ |
| 69 | +++ | ++++ |
| 70 | ++++ | ++++ |
| 71 | ++++ | ++++ |
| 72 | ++ | ++++ |
| 73 | +++ | ++++ |
| 74 | +++ | ++++ |
| 75 | ++++ | ++++ |
| 76 | ++++ | ++++ |
| 77 | ++++ | ++++ |
| 78 | ++++ | ++++ |
| 79 | +++ | ++++ |
| 80 | ++++ | ++++ |
| 81 | ++++ | ++++ |
| 82 | ++++ | ++++ |
| 83 | ++++ | ++++ |
| 84 | ++++ | +++ |
| 85 | ++++ | ++ |
| 86 | ++++ | ++++ |
| 87 | + | ++++ |
| 88 | ++++ | ++++ |
| 89 | ++ | ++++ |
| 90 | +++ | ++++ |
| 91 | ++ | ++++ |
| 92 | ++++ | ++++ |
| 93 | ++++ | ++++ |
| 94 | +++ | ++++ |
| 95 | ++++ | ++++ |
| 96 | ++++ | ++++ |
| 97 | ++++ | ++++ |
| 98 | ++++ | ++++ |
| 99 | +++ | ++++ |
| 100 | ++++ | ++++ |
| 101 | ++++ | ++++ |
| 102 | +++ | ++++ |
| 103 | +++ | ++++ |
| 104 | +++ | ++++ |
| 105 | +++ | ++++ |
| 106 | ++++ | ++++ |
| 107 | +++ | ++++ |
| 108 | ++++ | ++++ |
| 109 | ++++ | ++++ |
| 110 | ++++ | ++++ |
| 111 | ++++ | ++++ |
| 112 | ++++ | ++++ |
| 113 | +++ | ++++ |
| 114 | ++++ | ++++ |
| 115 | ++++ | ++++ |
| 116 | ++++ | ++++ |
| 117 | +++ | ++++ |
| 118 | +++ | ++++ |
| 119 | + | ++++ |
| 120 | +++ | ++++ |
| 121 | ++++ | ++++ |
| 122 | +++ | ++++ |
| 123 | +++ | ++++ |
| 124 | +++ | + |
| 125 | ++++ | ++++ |
| 126 | ++++ | ++++ |
| 127 | ++++ | ++++ |
| 128 | ++ | ++++ |
| 129 | +++ | +++ |
| 130 | ++++ | ++++ |
| 131 | +++ | ++++ |
| 132 | +++ | ++++ |
| 133 | +++ | ++++ |
| 134 | +++ | ++++ |
| 135 | ++++ | ++++ |
| 136 | ++++ | ++++ |
| 137 | ++++ | ++++ |
| 138 | ++++ | ++++ |
| 139 | ++++ | ++++ |
| 140 | +++ | ++++ |
| 141 | +++ | ++++ |
| 142 | +++ | ++++ |
| 143 | + | ++++ |
| 144 | +++ | +++ |
| 145 | ++ | +++ |
| 146 | ++ | ++ |
| 147 | ++ | +++ |
| 148 | +++ | ++++ |
| 149 | ++++ | +++ |
| 150 | ++ | + |
| 151 | +++ | ++++ |
| 152 | + | + |
| 153 | ++++ | ++++ |
| 154 | ++++ | ++++ |
| 155 | ++++ | ++++ |
| 156 | + | ++++ |
| 157 | + | ++++ |
| 158 | ++++ | ++++ |
| 159 | ++++ | ++++ |
| 160 | ++++ | ++++ |
| 161 | ++++ | ++++ |
| 162 | + | ++++ |
| 163 | +++ | +++ |
| 164 | +++ | +++ |
| 165 | + | +++ |
| 166 | ++ | ++++ |
| 167 | + | +++ |
| 168 | ++++ | ++++ |
| 169 | +++ | ++++ |
| 170 | + | ++++ |
| 171 | + | ++++ |
| 172 | ++++ | ++++ |
| 173 | + | ++++ |
| 174 | +++ | +++ |
| 175 | +++ | ++++ |
| 176 | + | ++++ |
| 177 | + | ++++ |
| 178 | + | ++++ |
| 179 | + | ++++ |
| 180 | + | ++++ |
| 181 | ++ | ++++ |
| 182 | + | ++ |
| 183 | ++ | ++ |

1-16. (canceled)

17. A compound of the formula: 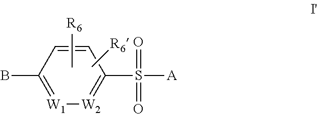
or a salt thereof, a co-crystal thereof, a prodrug thereof, or a solvate thereof,
wherein:
A is a moiety or the formula: 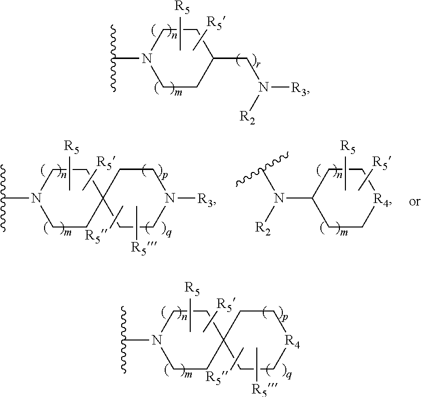
B is —NR₁R_{1′}; a branched or unbranched C₁₋₆ alkyl radical; a branched or unbranched C₁₋₆ haloalkyl radical; an heterocycloalkyl radical containing at least an O as heteroatom; or an heteroaryl radical containing at least one N as heteroatom optionally substituted with a C₁₋₆ haloalkyl radical;
one of W₁ or W₂ is —N— and the other is —CH—;
each of R₁ and R₁′ is independently H, a branched or unbranched C₁₋₆ alkyl radical or a moiety of the formula —C(O)R where R represents a C₁₋₆ alkyl radical, with the proviso that at least one of R₁ and R₁′ is not H; or
alternatively R₁ and R₁′ together with the nitrogen atom to which they are attached to form a 5- to 12-membered N-containing heterocyclic ring that (i) may further contain one or more additional heteroatoms selected from the group consisting of N, O or S, and (ii) is optionally substituted by one or more Rₐ radicals;
each of Rₐ is independently H; a branched or unbranched C₁₋₆ alkyl radical; halide; —CN;
a C₁₋₆ haloalkyl radical; —OH; a carbonyl group; or a C₁₋₆ alkoxy radical;
R₂ is H or a branched or unbranched C₁₋₆ alkyl radical;
R₃ is a branched or unbranched C₃₋₁₀ alkyl radical; an alkylaryl radical; or an alkylheteroaryl radical; wherein the aryl and the heteroaryl groups is optionally substituted by one or more R₃′ radicals;
R₃′ is a branched or unbranched C₁₋₆ alkyl radical, halide, —CN, a C₁₋₆ haloalkyl radical, —OH, or a C₁₋₆ alkoxy radical;
R₄ is CH(NR₂R₃) or NR₃;
each of R₅, R₅′, R₅″, and R₅′″ is independently H; a branched or unbranched C₁₋₆ alkyl radical; a C₁₋₆ alkoxy radical; halide; C₁₋₆ haloalkyl group; —OH, or —CN;
each of R₆ and R₆′ is independently H; a branched or unbranched C₁₋₆ alkyl radical; a C₁₋₆ alkoxy radical; a halogen atom; a C₁₋₆ haloalkyl group; —OH, or —CN;
n is 0, 1 or 2;
m is 0, 1 or 2;
p is 0, 1 or 2;
q is 0, 1 or 2;
r is 0, 1 or 2;
with the proviso that when R₃ is a benzyl group, then R_{3′} is not an a C₁₋₆ alkoxy radical.

18. The compound according to claim 17 of the formula: 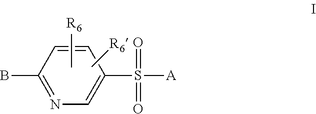
wherein A, B, R₆ and R₆′ are as defined in claim 17.

19. The compound according to claim 17, wherein A is a group selected from the group consisting of: 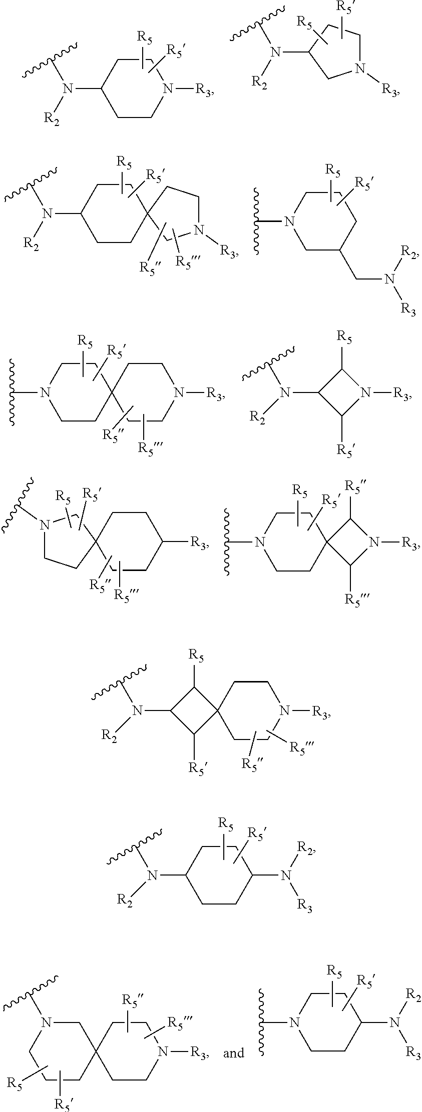
wherein R₂, R₃, R₅, R₅′, R₅″ and R₅′″ is as defined in claim 17.

20. The compound according to claim 17, wherein B is:
a branched or unbranched C₁₋₆ alkyl radical;
a tetrahydropyranyl group;
a pyridinyl group optionally substituted with a C₁₋₆ haloalkyl radical; or
—NR₁R_{1′}, wherein R₁ and R₁′ independently represent H; a branched or unbranched C₁₋₆ alkyl radical; or —C(O)R, wherein R is a C₁₋₆ alkyl radical, with the proviso that at least one of R₁ and R₁′ is not H, or
alternatively R₁ and R₁′ together with the nitrogen atom to which they are attached to form a moiety or the formula: 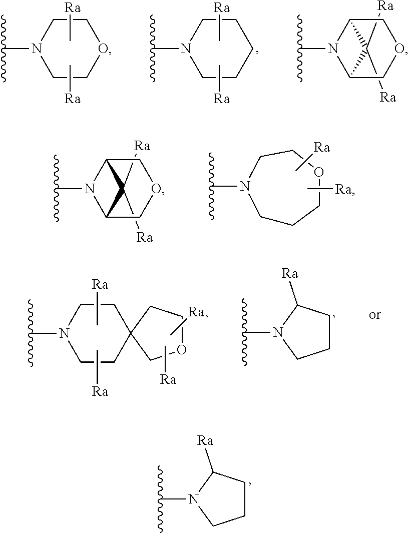
wherein Rₐ is as defined in claim 17.

21. The compound according to claim 17, wherein R₂ is H, methyl, ethyl or isopropyl.

22. The compound according to claim 17, wherein R₃ is branched or unbranched C₃₋₁₀ alkyl radical optionally substituted with one or more R₃′; benzyl optionally substituted with one or more R₃′ radicals; a pyridinylmethyl optionally substituted with one or more R₃′, or a phenethyl optionally substituted with one or more R₃′.

23. The compound according to claim 17, wherein R₃′ is halide; —CN; C₁₋₆ haloalkyl; —OH; or —OCH₃.

24. The compound according to claim 17, wherein each of R₅, R₅′, R₅″, and R₅′″ is independently H, or a branched or unbranched C₁₋₆ alkyl radical.

25. The compound according to claim 17, wherein said compound is of the formula: 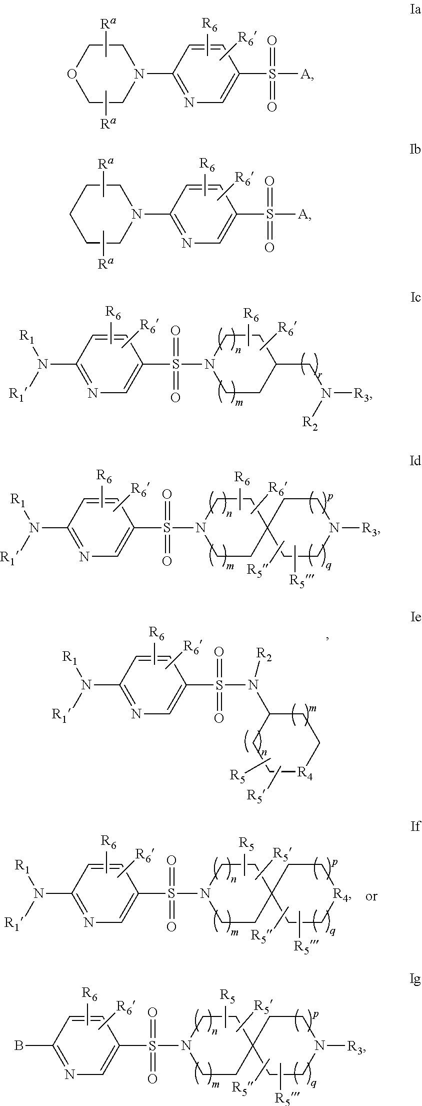
wherein A, B, Rₐ, R₁, R₁′, R₂, R₃, R₄, R₅, R₅′, R₅″, R₅′″, R₆, R₆′, n, m, p, q, and r are as defined in claim 17.

26. The compound according to claim 17, wherein said compound is selected from the group consisting of:
(S)—N-(1-Benzylpyrrolidin-3-yl)-6-morpholinopyridine-3-sulfonamide;
N-(1-Benzylpiperidin-4-yl)-6-morpholinopyridine-3-sulfonamide;
(R)—N-(1-Benzylpyrrolidin-3-yl)-6-morpholinopyridine-3-sulfonamide;
(S)—N-(1-Benzylpyrrolidin-3-yl)-N-methyl-6-morpholinopyridine-3-sulfonamide;
(R)—N-(1-Benzylpyrrolidin-3-yl)-N-methyl-6-morpholinopyridine-3-sulfonamide;
N-((3R,4S)-1-Benzyl-4-methylpyrrolidin-3-yl)-6-morpholinopyridine-3-sulfonamide;
N-(1-(4-Fluorobenzyl)piperidin-4-yl)-6-morpholinopyridine-3-sulfonamide;
N-(1-(3-Fluorobenzyl)piperidin-4-yl)-6-morpholinopyridine-3-sulfonamide;
N-(1-(3-Cyanobenzyl)piperidin-4-yl)-6-morpholinopyridine-3-sulfonamide;
N-(1-(4-Chlorobenzyl)piperidin-4-yl)-6-morpholinopyridine-3-sulfonamide;
N-(1-(4-Cyanobenzyl)piperidin-4-yl)-6-morpholinopyridine-3-sulfonamide;
4-(5-((9-Benzyl-3,9-diazaspiro[5.5]undecan-3-yl)sulfonyl)pyridin-2-yl)morpholine;
4-(5-((2-Benzyl-2,8-diazaspiro[4.5] decan-8-yl)sulfonyl)pyridin-2-yl)morpholine;
N-((3S,4R)-1-Benzyl-4-methylpyrrolidin-3-yl)-6-morpholinopyridine-3-sulfonamide;
N-Methyl-1-((6-morpholinopyridin-3-yl)sulfonyl)-N-phenethylpiperidin-4-amine;
N-Benzyl-N-methyl-1-((6-morpholinopyridin-3-yl)sulfonyl)piperidin-4-amine;
(S)—N-Benzyl-N-methyl-1-((6-morpholinopyridin-3-yl)sulfonyl)pyrrolidin-3-amine;
N-((1 r,4r)-4-(Benzyl(methyl)amino)cyclohexyl)-6-morpholinopyridine-3-sulfonamide;
N-((1 s,4s)-4-(Benzyl(methyl)amino)cyclohexyl)-6-morpholinopyridine-3-sulfonamide;
N—((S)-1-Benzylpyrrolidin-3-yl)-6-((2R,6S)-2,6-dimethylmorpholino)pyridine-3-sulfonamide;
(S)—N-(1-Benzylpyrrolidin-3-yl)-6-(dimethylamino)pyridine-3-sulfonamide;
(R)—N-(1-Benzylpyrrolidin-3-yl)-6-(dimethylamino)pyridine-3-sulfonamide;
N—((R)-1-Benzylpyrrolidin-3-yl)-6-((1S,4S)-2-oxa-5-azabicyclo[2.2.1]heptan-5-yl)pyridine-3-sulfonamide;
(R)—N-(1-Benzylpyrrolidin-3-yl)-5-fluoro-6-morpholinopyridine-3-sulfonamide;
(S)—N-(1-Benzylpyrrolidin-3-yl)-5-fluoro-6-morpholinopyridine-3-sulfonamide;
(R)—N-(1-Benzylpyrrolidin-3-yl)-6-(4-methoxypiperidin-1-yl)pyridine-3-sulfonamide;
N—((R)-1-Benzylpyrrolidin-3-yl)-6-((1R,5S)-3-oxa-8-azabicyclo[3.2.1]octan-8-yl)pyridine-3-sulfonamide;
N-((1r,4r)-4-((4-Fluorobenzyl)(methyl)amino)cyclohexyl)-6-morpholinopyridine-3-sulfonamide;
4-(5-((9-(3,3-Dimethylbutyl)-3,9-diazaspiro[5.5]undecan-3-yl)sulfonyl)pyridin-2-yl)morpholine;
N-((1r,4r)-4-(Benzyl(methyl)amino)cyclohexyl)-6-(dimethylamino)pyridine-3-sulfonamide;
N-((1 r,4r)-4-(Benzyl(methyl)amino)cyclohexyl)-6-(1-oxa-8-azaspiro[4.5]decan-8-yl)pyridine-3-sulfonamide;
N-((1 r,4r)-4-(Benzyl(methyl)amino)cyclohexyl)-6-(1,4-oxazepan-4-yl)pyridine-3-sulfonamide;
(R)-1-(6-Methoxypyridin-3-yl)-N-((1-((6-morpholinopyridin-3-yl)sulfonyl)piperidin-3-yl)methyl)methanamine;
(5)-1-(6-Methoxypyridin-3-yl)-N-((1-((6-morpholinopyridin-3-yl)sulfonyl)piperidin-3-yl)methyl)methanamine;
(S)—N-(1-(3-Fluorobenzyl)pyrrolidin-3-yl)-6-morpholinopyridine-3-sulfonamide;
(R)—N-(1-(4-Fluorobenzyl)pyrrolidin-3-yl)-6-morpholinopyridine-3-sulfonamide;
(R)—N-(1-(4-Cyanobenzyl)pyrrolidin-3-yl)-6-morpholinopyridine-3-sulfonamide;
(R)—N-(1-(3-Fluorobenzyl)pyrrolidin-3-yl)-6-morpholinopyridine-3-sulfonamide;
(R)—N-(1-(3-Fluorobenzyl)pyrrolidin-3-yl)-6-morpholinopyridine-3-sulfonamide;
(R)—N-(1-(3-Cyanobenzyl)pyrrolidin-3-yl)-6-morpholinopyridine-3-sulfonamide;
(S)—N-(1-(4-Fluorobenzyl)pyrrolidin-3-yl)-6-morpholinopyridine-3-sulfonamide;
(S)—N-(1-(3-Cyanobenzyl)pyrrolidin-3-yl)-6-morpholinopyridine-3-sulfonamide;
N-(1-Benzylazetidin-3-yl)-6-morpholinopyridine-3-sulfonamide;
(R)-6-Morpholino-N-(1-((6-(trifluoromethyl)pyridin-3-yl)methyl)pyrrolidin-3-yl)pyridine-3-sulfonamide;
4-(5-((8-Benzyl-2,8-diazaspiro[4.5] decan-2-yl)sulfonyl)pyridin-2-yl)morpholine;
N-(7-Benzyl-7-azaspiro[3.5]nonan-2-yl)-6-morpholinopyridine-3-sulfonamide;
4-(5-((2-Benzyl-2,7-diazaspiro[3.5]nonan-7-yl)sulfonyl)pyridin-2-yl)morpholine;
3-((9-((6-Morpholinopyridin-3-yl)sulfonyl)-3,9-diazaspiro[5.5]undecan-3-yl)methyl)benzonitrile;
(R)—N-(1-(4-Cyanobenzyl)pyrrolidin-3-yl)-N-isopropyl-6-morpholinopyridine-3-sulfonamide;
(S)—N-(1-(4-Cyanobenzyl)pyrrolidin-3-yl)-N-isopropyl-6-morpholinopyridine-3-sulfonamide;
N-((1 r,4r)-4-(Benzylamino)cyclohexyl)-6-morpholinopyridine-3-sulfonamide;
N-((1r,4r)-4-((3-Cyano-4-fluorobenzyl)amino)cyclohexyl)-6-morpholinopyridine-3-sulfonamide;
N-((1 r,4r)-4-((2-Fluorobenzyl)amino)cyclohexyl)-6-morpholinopyridine-3-sulfonamide;
N-((1 r,4r)-4-((3-Fluorobenzyl)amino)cyclohexyl)-6-morpholinopyridine-3-sulfonamide;
6-Morpholino-N-((1r,4r)-4-((pyridin-4-ylmethyl)amino)cyclohexyl)pyridine-3-sulfonamide;
N-((1r,4r)-4-((2-Fluorobenzyl)(methyl)amino)cyclohexyl)-6-morpholinopyridine-3-sulfonamide;
N-((1r,4r)-4-((3-Fluorobenzyl)(methyl)amino)cyclohexyl)-6-morpholinopyridine-3-sulfonamide;
N-((1 r,4r)-4-((3-Cyano-4-Fluorobenzyl)(methyl)amino)cyclohexyl)-6-morpholinopyridine-3-sulfonamide;
N-((1r,4r)-4-(Methyl(pyridin-3-ylmethyl)amino)cyclohexyl)-6-morpholinopyridine-3-sulfonamide;
N-((1r,4r)-4-(Methyl(pyridin-4-ylmethyl)amino)cyclohexyl)-6-morpholinopyridine-3-sulfonamide;
4-(5-((9-Benzyl-2,9-diazaspiro[5.5]undecan-2-yl)sulfonyl)pyridin-2-yl)morpholine;
2-Fluoro-4-((2-((6-morpholinopyridin-3-yl)sulfonyl)-2,9-diazaspiro[5.5]undecan-9-yl)methyl)benzonitrile;
4-(5-((9-(Pyridin-4-ylmethyl)-2,9-diazaspiro[5.5]undecan-2-yl)sulfonyl)pyridin-2-yl)morpholine;
(R)-5-((((1-((6-Morpholinopyridin-3-yl)sulfonyl)piperidin-3-yl)methyl)amino)methyl)pyridin-2-ol;
(5)-5-((((1-((6-Morpholinopyridin-3-yl)sulfonyl)piperidin-3-yl)methyl)amino)methyl)pyridin-2-ol;
3-(3,3-Dimethylbutyl)-9-((6-(trifluoromethyl)pyridin-3-yl)sulfonyl)-3,9-diazaspiro[5.5]undecane;
8-(3,3-Dimethylbutyl)-2-((6-(trifluoromethyl)pyridin-3-yl)sulfonyl)-2,8-diazaspiro[4.5]decane;
8-(5-((9-(3,3-Dimethylbutyl)-3,9-diazaspiro[5.5]undecan-3-yl)sulfonyl)pyridin-2-yl)-2-oxa-8-azaspiro[4.5]decane;
4-(5-((9-(3,3-Dimethylbutyl)-3,9-diazaspiro[5.5]undecan-3-yl)sulfonyl)pyridin-2-yl)thiomorpholine 1,1-dioxide;
3-((6-(4,4-Difluoropiperidin-1-yl)pyridin-3-yl)sulfonyl)-9-(3,3-dimethylbutyl)-3,9-diazaspiro[5.5]undecane;
8-(5-((9-(3,3-Dimethylbutyl)-3,9-diazaspiro[5.5]undecan-3-yl)sulfonyl)pyridin-2-yl)-1-oxa-8-azaspiro[4.5]decane;
7-(5-((9-(3,3-Dimethylbutyl)-3,9-diazaspiro[5.5]undecan-3-yl)sulfonyl)pyridin-2-yl)-2-oxa-7-azaspiro[3.5]nonane;
3-((6-(4,4-Difluoropiperidin-1-yl)pyridin-3-yl)sulfonyl)-9-isopentyl-3,9-diazaspiro[5.5]undecane;
3-Denzyl-9-((6-(4,4-difluoropiperidin-1-yl)pyridin-3-yl)sulfonyl)-3,9-diazaspiro[5.5]undecane;
4-(5-((2-(3,3-Dimethylbutyl)-2,9-diazaspiro[5.5]undecan-9-yl)sulfonyl)pyridin-2-yl)morpholine;
9-((6-(4,4-Difluoropiperidin-1-yl)pyridin-3-yl)sulfonyl)-2-(3,3-dimethylbutyl)-2,9-diazaspiro[5.5]undecane;
9-((6-(4,4-Difluoropiperidin-1-yl)pyridin-3-yl)sulfonyl)-2-isopentyl-2,9-diazaspiro[5.5]undecane;
8-(5-((2-(3,3-Dimethylbutyl)-2,9-diazaspiro[5.5]undecan-9-yl)sulfonyl)pyridin-2-yl)-1-oxa-8-azaspiro[4.5]decane;
4-(5-((8-(3,3-Dimethylbutyl)-2,8-diazaspiro[4.5] decan-2-yl)sulfonyl)pyridin-2-yl)morpholine;
2-((6-(4,4-Difluoropiperidin-1-yl)pyridin-3-yl)sulfonyl)-8-(3,3-dimethylbutyl)-2,8-diazaspiro[4.5]decane;
8-(5-((8-(3,3-Dimethylbutyl)-2,8-diazaspiro[4.5] decan-2-yl)sulfonyl)pyridin-2-yl)-1-oxa-8-azaspiro[4.5]decane;
8-Benzyl-2-((6-(4,4-difluoropiperidin-1-yl)pyridin-3-yl)sulfonyl)-2,8-diazaspiro[4.5]decane;
2-((6-(4,4-Difluoropiperidin-1-yl)pyridin-3-yl)sulfonyl)-8-((tetrahydro-2H-pyran-4-yl)methyl)-2,8-diazaspiro[4.5]decane;
9-((6-(4,4-Difluoropiperidin-1-yl)pyridin-3-yl)sulfonyl)-2-((tetrahydro-2H-pyran-4-yl)methyl)-2,9-diazaspiro[5.5]undecane;
9-((6-(4,4-Difluoropiperidin-1-yl)pyridin-3-yl)sulfonyl)-2-neopentyl-2,9-diazaspiro[5.5]undecane;
N-Benzyl-1-((6-(4,4-difluoropiperidin-1-yl)pyridin-3-yl)sulfonyl)-N-methylpiperidin-4-amine;
1-((6-(1-Oxa-8-azaspiro[4.5] decan-8-yl)pyridin-3-yl)sulfonyl)-N-benzyl-N-methylpiperidin-4-amine;
4-(5-((9-(3,3-Dimethylbutyl)-2,9-diazaspiro[5.5]undecan-2-yl)sulfonyl)pyridin-2-yl)morpholine;
N-((1r,4r)-4-((3,3-Dimethylbutyl)(methyl)amino)cyclohexyl)-6-morpholinopyridine-3-sulfonamide;
3-(2-Isopropoxyethyl)-9-((6-(trifluoromethyl)pyridin-3-yl)sulfonyl)-3,9-diazaspiro[5.5]undecane;
3-Isobutyl-9-((6-(trifluoromethyl)pyridin-3-yl)sulfonyl)-3,9-diazaspiro[5.5]undecane;
9-(3,3-Dimethylbutyl)-2-((6-(trifluoromethyl)pyridin-3-yl)sulfonyl)-2,9-diazaspiro[5.5]undecane;
3-Isopentyl-9-((6-(trifluoromethyl)pyridin-3-yl)sulfonyl)-3,9-diazaspiro[5.5]undecane;
5-((9-(3,3-Dimethylbutyl)-2,9-diazaspiro[5.5]undecan-2-yl)sulfonyl)-N-methylpyridin-2-amine;
8-(5-((9-(3,3-Dimethylbutyl)-2,9-diazaspiro[5.5]undecan-2-yl)sulfonyl)pyridin-2-yl)-2-oxa-8-azaspiro[4.5]decane;
2-((6-(4,4-Difluoropiperidin-1-yl)pyridin-3-yl)sulfonyl)-9-(3,3-dimethylbutyl)-2,9-diazaspiro[5.5]undecane;
5-((9-(3,3-Dimethylbutyl)-2,9-diazaspiro[5.5]undecan-2-yl)sulfonyl)-N,N-dimethylpyridin-2-amine;
8-(5-((9-(3,3-Dimethylbutyl)-2,9-diazaspiro[5.5]undecan-2-yl)sulfonyl)pyridin-2-yl)-1-oxa-8-azaspiro[4.5]decane;
3-((6-(4,4-Difluoropiperidin-1-yl)pyridin-3-yl)sulfonyl)-9-neopentyl-3,9-diazaspiro[5.5]undecane;
1-((6-(4,4-Difluoropiperidin-1-yl)pyridin-3-yl)sulfonyl)-N-methyl-N-neopentylpiperidin-4-amine;
2-((6-(4,4-Difluoropiperidin-1-yl)pyridin-3-yl)sulfonyl)-9-((tetrahydro-2H-pyran-4-yl)methyl)-2,9-diazaspiro[5.5]undecane;
N-(5-((9-(3,3-Dimethylbutyl)-2,9-diazaspiro[5.5]undecan-2-yl)sulfonyl)pyridin-2-yl)-N-methylacetamide;
N-(5-((9-(3,3-Dimethylbutyl)-3,9-diazaspiro[5.5]undecan-3-yl)sulfonyl)pyridin-2-yl)-N-methylacetamide;
N-(5-((9-(3,3-Dimethylbutyl)-2,9-diazaspiro[5.5]undecan-2-yl)sulfonyl)pyridin-2-yl)-N-methylpropionamide;
N-(5-((9-(3,3-Dimethylbutyl)-3,9-diazaspiro[5.5]undecan-3-yl)sulfonyl)pyridin-2-yl)-N-methylpropionamide;
N-(5-((9-(3,3-Dimethylbutyl)-2,9-diazaspiro[5.5]undecan-2-yl)sulfonyl)pyridin-2-yl)propionamide;
N-(5-((9-(3,3-Dimethylbutyl)-2,9-diazaspiro[5.5]undecan-2-yl)sulfonyl)pyridin-2-yl)acetamide;
9-(3,3-Dimethylbutyl)-2-((6-methylpyridin-3-yl)sulfonyl)-2,9-diazaspiro[5.5]undecane;
3-(3,3-Dimethylbutyl)-9-((6-ethylpyridin-3-yl)sulfonyl)-3,9-diazaspiro[5.5]undecane;
3-(3,3-Dimethylbutyl)-9-((6-methylpyridin-3-yl)sulfonyl)-3,9-diazaspiro[5.5]undecane;
9-(3,3-Dimethylbutyl)-2-((6-ethylpyridin-3-yl)sulfonyl)-2,9-diazaspiro[5.5]undecane;
2-([2,4′-Bipyridin]-5-ylsulfonyl)-9-(3,3-dimethylbutyl)-2,9-diazaspiro[5.5]undecane;
3-([2,4′-Bipyridin]-5-ylsulfonyl)-9-(3,3-dimethylbutyl)-3,9-diazaspiro[5.5]undecane;
2-([2,4′-Bipyridin]-5-ylsulfonyl)-8-(3,3-dimethylbutyl)-2,8-diazaspiro[4.5]decane;
9-([2,4′-Bipyridin]-5-ylsulfonyl)-2-(3,3-dimethylbutyl)-2,9-diazaspiro[5.5]undecane;
9-(3,3-Dimethylbutyl)-2-((6-(tetrahydro-2H-pyran-4-yl)pyridin-3-yl)sulfonyl)-2,9-diazaspiro[5.5]undecane;
3-(3,3-Dimethylbutyl)-9-((6-(tetrahydro-2H-pyran-4-yl)pyridin-3-yl)sulfonyl)-3,9-diazaspiro[5.5]undecane;
4-(5-((9-(3,3-Dimethylbutyl)-2,9-diazaspiro[5.5]undecan-2-yl)sulfonyl)pyridin-2-yl)thiomorpholine 1,1-dioxide;
N-((1 r,4r)-4-((4-Fluorobenzyl)(methyl)amino)cyclohexyl)-5-morpholinopyridine-2-sulfonamide;
4-(5-((9-(3,3-Dimethylbutyl)-2,9-diazaspiro[5.5]undecan-2-yl)sulfonyl)pyridin-2-yl)morpholin-3-one;
1-(5-((9-(3,3-Dimethylbutyl)-2,9-diazaspiro[5.5]undecan-2-yl)sulfonyl)pyridin-2-yl)pyrrolidin-2-one;
1-(5-((9-(3,3-Dimethylbutyl)-2,9-diazaspiro[5.5]undecan-2-yl)sulfonyl)pyridin-2-yl)piperidin-2-one;
3-(5-((9-(3,3-Dimethylbutyl)-2,9-diazaspiro[5.5]undecan-2-yl)sulfonyl)pyridin-2-yl)oxazolidin-2-one;
3-(5-((4-(Methyl(neopentyl)amino)piperidin-1-yl)sulfonyl)pyridin-2-yl)oxazolidin-2-one;
3-Fluoro-4-((2-((6-(2-oxooxazolidin-3-yl)pyridin-3-yl)sulfonyl)-2,9-diazaspiro[5.5]undecan-9-yl)methyl)benzonitrile;
1-(5-((9-((Tetrahydro-2H-pyran-4-yl)methyl)-2,9-diazaspiro[5.5]undecan-2-yl)sulfonyl)pyridin-2-yl)pyrrolidin-2-one;
3-(6-((9-((Tetrahydro-2H-pyran-4-yl)methyl)-2,9-diazaspiro[5.5]undecan-2-yl)sulfonyl)pyridin-3-yl)oxazolidin-2-one;
3-(5-((9-((Tetrahydro-2H-pyran-4-yl)methyl)-3,9-diazaspiro[5.5]undecan-3-yl)sulfonyl)pyridin-2-yl)oxazolidin-2-one;
3-(5-((4-(Methyl((tetrahydro-2H-pyran-4-yl)methyl)amino)piperidin-1-yl)sulfonyl)pyridin-2-yl)oxazolidin-2-one;
9-Benzyl-2-((6-(4,4-difluoropiperidin-1-yl)pyridin-3-yl)sulfonyl)-2,9-diazaspiro[5.5]undecane;
4-(5-((9-(3,3-Dimethylbutyl)-3,9-diazaspiro[5.5]undecan-3-yl)sulfonyl)pyridin-2-yl)morpholin-3-one;
1-(5-((9-(3,3-Dimethylbutyl)-3,9-diazaspiro[5.5]undecan-3-yl)sulfonyl)pyridin-2-yl)piperidin-2-one;
1-(5-((9-(3,3-Dimethylbutyl)-3,9-diazaspiro[5.5]undecan-3-yl)sulfonyl)pyridin-2-yl)pyrrolidin-2-one;
3-(5-((9-(3,3-Dimethylbutyl)-3,9-diazaspiro[5.5]undecan-3-yl)sulfonyl)pyridin-2-yl)oxazolidin-2-one;
3-(5-((9-Benzyl-3,9-diazaspiro[5.5]undecan-3-yl)sulfonyl)pyridin-2-yl)oxazolidin-2-one;
1-(5-((2-(3,3-Dimethylbutyl)-2,9-diazaspiro[5.5]undecan-9-yl)sulfonyl)pyridin-2-yl)pyrrolidin-2-one;
3-(5-((2-(3,3-Dimethylbutyl)-2,9-diazaspiro[5.5]undecan-9-yl)sulfonyl)pyridin-2-yl)oxazolidin-2-one;
3-(5-((8-(3,3-Dimethylbutyl)-2,8-diazaspiro[4.5] decan-2-yl)sulfonyl)pyridin-2-yl)oxazolidin-2-one;
1-(5-((8-(3,3-Dimethylbutyl)-2,8-diazaspiro[4.5] decan-2-yl)sulfonyl)pyridin-2-yl)pyrrolidin-2-one;
3-(5-((9-(3,3-Dimethylbutyl)-3,9-diazaspiro[5.5]undecan-3-yl)sulfonyl)-3-fluoropyridin-2-yl)oxazolidin-2-one;
1-(5-((9-(3,3-Dimethylbutyl)-3,9-diazaspiro[5.5]undecan-3-yl)sulfonyl)-3-fluoropyridin-2-yl)pyrrolidin-2-one;
3-(5-((4-(Benzyl(methyl)amino)piperidin-1-yl)sulfonyl)pyridin-2-yl)oxazolidin-2-one;
3-(5-((9-Neopentyl-3,9-diazaspiro[5.5]undecan-3-yl)sulfonyl)pyridin-2-yl)oxazolidin-2-one;
3-(5-((8-Benzyl-2,8-diazaspiro[4.5] decan-2-yl)sulfonyl)pyridin-2-yl)oxazolidin-2-one;
3-(5-((2-(2-Fluorobenzyl)-2,9-diazaspiro[5.5]undecan-9-yl)sulfonyl)pyridin-2-yl)oxazolidin-2-one;
3-(5-((2-(2,5-Difluorobenzyl)-2,9-diazaspiro[5.5]undecan-9-yl)sulfonyl)pyridin-2-yl)oxazolidin-2-one;
3-(5-((2-Benzyl-2,9-diazaspiro[5.5]undecan-9-yl)sulfonyl)pyridin-2-yl)oxazolidin-2-one;
1-(5-((4-(Benzyl(methyl)amino)piperidin-1-yl)sulfonyl)pyridin-2-yl)pyrrolidin-2-one;
3-(5-((2-((Tetrahydro-2H-pyran-4-yl)methyl)-2,9-diazaspiro[5.5]undecan-9-yl)sulfonyl)pyridin-2-yl)oxazolidin-2-one;
3-(5-((2-Neopentyl-2,9-diazaspiro[5.5]undecan-9-yl)sulfonyl)pyridin-2-yl)oxazolidin-2-one;
2-Fluoro-4-((9-((6-(2-oxooxazolidin-3-yl)pyridin-3-yl)sulfonyl)-2,9-diazaspiro[5.5]undecan-2-yl)methyl)benzonitrile;
2-Fluoro-5-((9-((6-(2-oxooxazolidin-3-yl)pyridin-3-yl)sulfonyl)-2,9-diazaspiro[5.5]undecan-2-yl)methyl)benzonitrile;
3-(5-((8-((Tetrahydro-2H-pyran-4-yl)methyl)-2,8-diazaspiro[4.5] decan-2-yl)sulfonyl)pyridin-2-yl)oxazolidin-2-one;
3-(6-((2-(3,3-Dimethylbutyl)-2,9-diazaspiro[5.5]undecan-9-yl)sulfonyl)pyridin-3-yl)oxazolidin-2-one;
3-(5-((9-Benzyl-2,9-diazaspiro[5.5]undecan-2-yl)sulfonyl)pyridin-2-yl)oxazolidin-2-one;
3-(5-((9-(2,5-Difluorobenzyl)-3,9-diazaspiro[5.5]undecan-3-yl)sulfonyl)pyridin-2-yl)oxazolidin-2-one;
2-Fluoro-5-((9-((6-(2-oxooxazolidin-3-yl)pyridin-3-yl)sulfonyl)-3,9-diazaspiro[5.5]undecan-3-yl)methyl)benzonitrile;
3-(5-((9-((Tetrahydro-2H-pyran-4-yl)methyl)-2,9-diazaspiro[5.5]undecan-2-yl)sulfonyl)pyridin-2-yl)oxazolidin-2-one;
3-(5-((9-(2-Fluorobenzyl)-2,9-diazaspiro[5.5]undecan-2-yl)sulfonyl)pyridin-2-yl)oxazolidin-2-one;
3-(5-((9-(2,5-Difluorobenzyl)-2,9-diazaspiro[5.5]undecan-2-yl)sulfonyl)pyridin-2-yl)oxazolidin-2-one;
2-Fluoro-5-((2-((6-(2-oxooxazolidin-3-yl)pyridin-3-yl)sulfonyl)-2,9-diazaspiro[5.5]undecan-9-yl)methyl)benzonitrile;
3-(5-((8-(2,5-Difluorobenzyl)-2,8-diazaspiro[4.5] decan-2-yl)sulfonyl)pyridin-2-yl)oxazolidin-2-one;
3-(5-((8-(2-Fluorobenzyl)-2,8-diazaspiro[4.5] decan-2-yl)sulfonyl)pyridin-2-yl)oxazolidin-2-one;
2-Fluoro-4-((2-((6-(2-oxooxazolidin-3-yl)pyridin-3-yl)sulfonyl)-2,8-diazaspiro[4.5]decan-8-yl)methyl)benzonitrile;
2-Fluoro-5-((2-((6-(2-oxooxazolidin-3-yl)pyridin-3-yl)sulfonyl)-2,8-diazaspiro[4.5]decan-8-yl)methyl)benzonitrile;
3-(5-((9-(2-Fluorobenzyl)-3,9-diazaspiro[5.5]undecan-3-yl)sulfonyl)pyridin-2-yl)oxazolidin-2-one;
N-((1r,4r)-4-(Benzyl(methyl)amino)cyclohexyl)-6-(2-oxooxazolidin-3-yl)pyridine-3-sulfonamide;
1-((6-(4,4-Difluoropiperidin-1-yl)pyridin-3-yl)sulfonyl)-N-(3,3-dimethylbutyl)-N-methylpiperidin-4-amine;
1-((6-(4,4-Difluoropiperidin-1-yl)pyridin-3-yl)sulfonyl)-N-methyl-N-((tetrahydro-2H-pyran-4-yl)methyl)piperidin-4-amine;
N-Benzyl-1-((6-(4,4-difluoropiperidin-1-yl)pyridin-3-yl)sulfonyl)-N-methylpiperidin-4-amine;
4-(((1-((6-(4,4-Difluoropiperidin-1-yl)pyridin-3-yl)sulfonyl)piperidin-4-yl)(methyl)amino)methyl)-3-fluorobenzonitrile;
1-((6-(4,4-Difluoropiperidin-1-yl)pyridin-3-yl)sulfonyl)-N-(2-fluorobenzyl)-N-methylpiperidin-4-amine;
4-(((1-((6-(4,4-Difluoropiperidin-1-yl)pyridin-3-yl)sulfonyl)piperidin-4-yl)(methyl)amino)methyl)-2-fluorobenzonitrile;
N-(2,5-Difluorobenzyl)-1-((6-(4,4-difluoropiperidin-1-yl)pyridin-3-yl)sulfonyl)-N-methylpiperidin-4-amine;
N-((1 r,4r)-4-(Benzyl(methyl)amino)cyclohexyl)-6-(4,4-difluoropiperidin-1-yl)pyridine-3-sulfonamide;
N-((1 r,4r)-4-(Benzyl(methyl)amino)cyclohexyl)-6-(1-oxa-8-azaspiro[4.5]decan-8-yl)pyridine-3-sulfonamide;
N-((1 r,4r)-4-(Methyl((tetrahydro-2H-pyran-4-yl)methyl)amino)cyclohexyl)-6-(1-oxa-8-azaspiro[4.5]decan-8-yl)pyridine-3-sulfonamide;
N-((1 r,4r)-4-((2-Fluorobenzyl)(methyl)amino)cyclohexyl)-6-(1-oxa-8-azaspiro[4.5]decan-8-yl)pyridine-3-sulfonamide;
N-((1 r,4r)-4-((2,5-Difluorobenzyl)(methyl)amino)cyclohexyl)-6-(2-oxa-8-azaspiro[4.5]decan-8-yl)pyridine-3-sulfonamide;
N-((1 r,4r)-4-((3,4-Difluorobenzyl)(methyl)amino)cyclohexyl)-6-(2-oxa-8-azaspiro[4.5]decan-8-yl)pyridine-3-sulfonamide;
N-((1 r,4r)-4-((3,3-Dimethylbutyl)(methyl)amino)cyclohexyl)-6-(2-oxa-8-azaspiro[4.5]decan-8-yl)pyridine-3-sulfonamide;
N-((1 r,4r)-4-(Methyl(neopentyl)amino)cyclohexyl)-6-(2-oxa-8-azaspiro[4.5]decan-8-yl)pyridine-3-sulfonamide and
N-((1 r,4r)-4-(Methyl((tetrahydro-2H-pyran-4-yl)methyl)amino)cyclohexyl)-6-(2-oxa-8-azaspiro[4.5]decan-8-yl)pyridine-3-sulfonamide,
or a salt thereof, a co-crystal thereof, a prodrug thereof, or a solvate thereof.

27. A process for producing a compound of claim 17 of the formula: 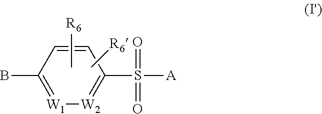
said process comprising reacting a compound of the formula: 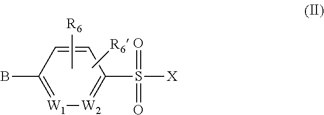
with a compound of the formula: H-A (III) under condition sufficient to produce said compound of Formula I′, wherein A, B, W₁, W₂, R₁, R₁′, R₆ and R₆′ are as defined in claim 17, and X is a leaving group.

28. A pharmaceutical composition comprising a compound of claim 17 or a salt thereof, a co-crystal thereof, a prodrug thereof, or a solvate thereof.

29. The pharmaceutical composition of claim 28, wherein said compound comprises an enantiomer mixture, a diastereomer mixture, or a racemic mixture.

30. The pharmaceutical composition according to claim 28, wherein said compound comprises an enantiomerically enriched mixture.

31. The pharmaceutical composition according to claim 28, wherein said compound comprises a diastereomerically enriched mixture.

32. A method for treating a clinical condition associated with a disorder mediated by a sigma receptor, said method comprising administering to a subject in need of such a treatment a therapeutically effective amount of a compound of claim 17.

33. The method of claim 32, wherein said clinical condition associated with a disorder mediated by a sigma receptor comprises pain or a CNS disorder or disease.

34. The method of claim 32, wherein said pain is selected from the group consisting of neuropathic pain, inflammatory pain, chronic pain, and any other pain associated with allodynia and/or hyperalgesia.

35. The method of claim 32, wherein said CNS disorder or disease is selected from the group consisting of addiction to drug or chemical substance, anxiety, attention-deficit-/hyperactivity disorder (ADHD), autism spectrum disorder, catalepsy, cognition disorder, learning, memory and attention deficit, depression, encephalitis, epilepsy, headache disorder, insomnia, locked-in-syndrome, meningitis, migraine, multiple sclerosis (MS), leukodystrophies, amyotrophic lateral sclerosis (ALS), myelopathy, narcolepsy, neurodegenerative disease, traumatic brain injury, Alzheimer disease, Gaucher's disease, Huntington disease, Parkinson disease, Tourette's syndrome, psychotic condition, bipolar disorder, schizophrenia, and paranoia.

36. The method of claim 35, wherein said addiction to drug or chemical substance comprises addiction to cocaine, amphetamine, ethanol, or nicotine.
